(19) United States
(12) Patent Application Publication
(10) Pub. No.: US 2023/0414788 A1
(43) Pub. Date: Dec. 28, 2023

(54) FORMULATIONS FOR SUPRACHOROIDAL ADMINISTRATION SUCH AS GEL FORMULATIONS

(71) Applicant: REGENXBIO Inc., Rockville, MD (US)

(72) Inventors: Jared Bee, Gaithersburg, MD (US); Tristan James Marshall, Potomac, MD (US); Sherri Van Everen, Menlo Park, CA (US); Stephen Joseph Pakola, Irvington, NY (US); Nicholas Alexander Piers Sascha Buss, Rockville, MD (US); Anthony Ray O'Berry, Clarksburg, MD (US); Jesse I. Yoo, Atlanta, GA (US); Ewa Budzynski, Deforest, WI (US)

(21) Appl. No.: 18/030,616

(22) PCT Filed: Oct. 6, 2021
(86) PCT No.: PCT/US2021/053818

§ 371 Date: Apr. 6, 2023

Related U.S. Application Data
(60) Provisional application No. 63/147,584, filed on Feb. 9, 2021.
(60) Provisional application No. 63/088,886, filed on Oct. 7, 2020.

(51) Int. Cl. A61K 48/00; A61K 47/02; A61K 47/10; A61K 47/26; A61K 9/00; C12N 15/86; C07K 16/22; A61P 27/02

(52) U.S. Cl. CPC A61K 48/0075; A61K 48/0041; A61K 47/02; A61K 47/10; A61K 47/26; A61K 48/0091; A61K 9/0024; C12N 15/86; C07K 16/22; A61P 27/02

(57) ABSTRACT

Provided herein are pharmaceutical compositions for administration to a suprachoroidal space of an eye of a subject. The pharmaceutical compositions can include a recombinant adeno-associated virus (AAV) encoding a transgene. Also provided herein are methods for treating or preventing a disease in a subject by administering a therapeutically effective amount of the pharmaceutical compositions to the subject in need.

### PRIORITY

This application claims the benefit of priority to U.S. Ser. No. 63/088,886, filed Oct. 7, 2020, and U.S. Ser. No. 63/147,584, filed Feb. 9, 2021, each of which is incorporated herein by reference in its entirety.

### REFERENCE TO SEQUENCE LISTING SUBMITTED ELECTRONICALLY

This application incorporates by reference a Sequence Listing submitted with this application as text file entitled “12656-143-228_Sequence_Listing.txt” created on Sep. 30, 2021 and having a size of 107,121 bytes.

### 1. BACKGROUND OF THE INVENTION

The human eye is a highly intricate and highly developed sensory organ, which is prone to a host of diseases and disorders. About 285 million people in the world are visually impaired, of whom 39 million are blind and 246 million have moderate to severe visual impairment (World Health Organization, 2012, “Global Data On Visual Impairments 2010,” Geneva: World Health Organization). Some of the leading causes of blindness are cataract (47%), glaucoma (12%), age-related macular degeneration (AMD) (9%), and diabetic retinopathy (5%) (World Health Organization, 2007, “Global Initiative For The Elimination Of Avoidable Blindness: Action Plan 2006-2011,” Geneva: World Health Organization).

Gene therapy has been employed in treating certain eye diseases (see, e.g. International Patent Application No. PCT/US2017/027650 (International Publication No. WO 2017/181021 A1)). Adeno-associated viruses (AAV) are an attractive tool for gene therapy due to properties of non-pathogenicity, broad host and cell type tropism range of infectivity, including both dividing and non-dividing cells, and ability to establish long-term transgene expression (e.g., Gonçalves, 2005, Virology Journal, 2:43).

Current methods used for ocular gene therapy (e.g., by intravitreous or subretinal administrations) are invasive and have serious setbacks, such as, increased risk of cataract, retinal detachment, and separation of photoreceptors from the retinal pigment epithelium (RPE) in the fovea. There is a significant unmet medical need for therapies that improve or eliminate the setbacks from current ocular gene therapy.

Adeno-associated virus (AAV), a member of the Parvoviridae family designated Dependovirus, is a small nonenveloped, icosahedral virus with single-stranded linear DNA genomes of approximately 4.7-kilobases (kb) to 6 kb. The properties of non-pathogenicity, broad host and cell type tropism range of infectivity, including both dividing and non-dividing cells, and ability to establish long-term transgene expression make AAV an attractive tool for gene therapy (e.g., Gonçalves, 2005, Virology Journal, 2:43).

Construct II is being investigated as a treatment delivered by injection into the suprachoroidal space. The suprachoroidal space (SCS) is a region between the sclera and the choroid that expands upon injection of the drug solution (Habot-Wilner, 2019). The SCS space recovers to its pre-injection size as the injected solution is cleared by physiologic processes. The drug solution diffuses within SCS and is absorbed into adjacent tissues. Capillaries in the choroid are permeable to low molecular weight osmolytes. The present disclosure addresses an unmet need of providing pharmaceutical compositions that lead to longer residence time in the suprachoroidal space, and consequently improved efficacy.

### 2. SUMMARY OF THE INVENTION

In one aspect, provided herein is a pharmaceutical composition suitable for administration to the suprachoroidal space (SCS) of an eye of a human subject, wherein the pharmaceutical composition comprises a recombinant adeno-associated virus (AAV) vector comprising an expression cassette encoding a transgene, and wherein the pharmaceutical has a viscosity and/or higher elastic modulus that increases with increasing temperature. In some embodiments, the composition has a gelation temperature of about 27-32° C. In some embodiments, the composition has a gelation time of about 15-90 seconds. In some embodiments, the composition has a viscosity of about 183 mPas at 5° C. as measured at a shear rate of about 1 s⁻¹ to about 1000 s⁻¹. In some embodiments, the composition has a viscosity of less than about 183 mPas at 5° C. as measured at a shear rate of about 1 s⁻¹ to 1000 s⁻¹. In some embodiments, wherein the composition has a viscosity of about 183 mPas at 20° C. as measured at a shear rate of at about 1 s⁻¹ to 1000 s⁻¹. In some embodiments, the composition has a viscosity of less than about 183 mPas at 20° C. as measured at a shear rate of at about 1 s⁻¹ to 1000 s⁻¹

In some embodiments, the elastic modulus of a pharmaceutical composition provided herein at under 27° C. is less than about or about 0.1 Pa, less than about or about 0.01 Pa, less than about or about 0.001 Pa or zero. In some embodiments, the elastic modulus of a pharmaceutical composition provided herein at about 32° C.-35° C. is about or at least about 0.1 Pa, about or at least about 1 Pa, about or at least about 10 Pa, about or at least about 100 Pa, about or at least about 1000 Pa, about or at least about 10,000 Pa or about or at least about 100,000 Pa.

In some embodiments, the clearance time after suprachoroidal administration is equal to or greater than the clearance time of a reference pharmaceutical composition after suprachoroidal administration, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a lower viscosity and/or lower elastic modulus than the pharmaceutical composition at about 32-35° C.

In some embodiments, a circumferential spread after suprachoroidal administration is smaller as compared to a circumferential spread of a reference pharmaceutical composition after suprachoroidal administration, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a lower viscosity and/or lower elastic modulus than the pharmaceutical composition at about 32-35° C. In some embodiments, the circumferential spread after suprachoroidal administration is smaller by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

In some embodiments, a thickness at a site of injection after suprachoroidal administration is equal to or higher as compared to a thickness at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a lower viscosity and/or lower elastic modulus than the pharmaceutical composition at about 32-35° C.

In some embodiments, an expression level of the transgene is detected in the eye for a longer period of time after suprachoroidal administration as compared to a period of time that an expression level of the transgene is detected in the eye after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a lower viscosity and/or lower elastic modulus than the pharmaceutical composition at about 32-35° C.

In some embodiments, the concentration of the transgene in the eye after suprachoroidal administration is equal to or higher as compared to the concentration of the transgene in the eye suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a lower viscosity and/or lower elastic modulus than the pharmaceutical composition at about 32-35° C.

In some embodiments, the rate of transduction at a site of injection after suprachoroidal administration is equal to or higher as compared to the rate of transduction at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a lower viscosity and/or lower elastic modulus than the pharmaceutical composition at about 32-35° C.

In some embodiments, a level of VEGF-induced vasodilation and/or vascular leakage after suprachoroidal administration is equal to or decreased as compared to a level of VEGF-induced vasodilation and/or vascular leakage after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a lower viscosity and/or lower elastic modulus than the pharmaceutical composition at about 32-35° C.

In some embodiments, the viscosity and/or elastic modulus of the pharmaceutical composition and the viscosity and/or elastic modulus of the reference pharmaceutical composition is measured at the same shear rate. In some embodiments, the viscosity and/or elastic modulus of the pharmaceutical composition is measured at a shear rate of at least about 1,000 s⁻¹, 2,000 s⁻¹, 3,000 s⁻¹, 4,000 s⁻¹, 5,000 s⁻¹, 6,000 s⁻¹, 7,000 s⁻¹, 8,000 s⁻¹, 9,000 s⁻¹, 10,000 s⁻¹, 15,000 s⁻¹, 20,000 s⁻¹, or 30,000 s⁻¹.

In some embodiments, the recombinant AAV is Construct II. In some embodiments, the transgene is an anti-human vascular endothelial growth factor (anti-VEGF) antibody. In some embodiments, the recombinant AAV comprises components from one or more adeno-associated virus serotypes selected from the group consisting of AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the recombinant AAV is AAV8. In some embodiments, the recombinant AAV is AAV9.

In some embodiments, the pharmaceutical composition comprises sucrose. In some embodiments, the pharmaceutical composition does not comprise sucrose.

In some embodiments, the clearance time after suprachoroidal administration of the pharmaceutical composition is greater by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or at least 500%. In some embodiments, the clearance time after suprachoroidal administration of the pharmaceutical composition is of about 30 minutes to about 20 hours, about 2 hours to about 20 hours, about 30 minutes to about 24 hours, about 1 hour to about 2 hours, about 30 minutes to about 90 days, about 30 minutes to about 60 days, about 30 minutes to about 30 days, about 30 minutes to about 21 days, about 30 minutes to about 14 days, about 30 minutes to about 7 days, about 30 minutes to about 3 days, about 30 minutes to about 2 days, about 30 minutes to about 1 day, about 4 hours to about 90 days, about 4 hours to about 60 days, about 4 hours to about 30 days, about 4 hours to about 21 days, about 4 hours to about 14 days, about 4 hours to about 7 days, about 4 hours to about 3 days, about 4 hours to about 2 days, about 4 hours to about 1 day, about 4 hours to about 8 hours, about 4 hours to about 16 hours, about 4 hours to about 20 hours, about 1 day to about 90 days, about 1 day to about 60 days, about 1 day to about 30 days, about 1 day to about 21 days, about 1 day to about 14 days, about 1 day to about 7 days, about 1 day to about 3 days, about 2 days to about 90 days, about 3 days to about 90 days, about 3 days to about 60 days, about 3 days to about 30 days, about 3 days to about 21 days, about 3 days to about 14 days, or about 3 days to about 7 days.

In some embodiments, the clearance time after suprachoroidal administration of the pharmaceutical composition is not prior to about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days. In some embodiments, the clearance time of the reference pharmaceutical composition after suprachoroidal administration is of at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, or 14 days. In some embodiments, the clearance time is from the SCS or from the eye.

In some embodiments, the thickness at the site of injection after suprachoroidal administration of the pharmaceutical composition is higher by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%. In some embodiments, the thickness at the site of injection after suprachoroidal administration of the pharmaceutical composition is about 500 μm to about 3.0 mm, 750 μm to about 2.8 mm, about 750 μm to about 2.5 mm, about 750 μm to about 2 mm, or about 1 mm to about 2 mm.

In some embodiments, the thickness at the site of injection after suprachoroidal administration of the pharmaceutical composition is of at least about 50 μm, 100 μm, 200 μm, 300 μm, 400 μm, 500 μm, 600 μm, 700 μm, 800 μm, 900 μm, 1000 μm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm. In some embodiments, the thickness at the site of injection after the suprachoroidal administration of the reference pharmaceutical composition is of at most about 1 nm, 5 nm, 10 nm, 25 nm, 50 nm, 100 nm, 200 nm, 300 nm, 400 nm, 500 nm, 600 nm, 700 nm, 800 nm, 900 nm, 1 μm, 5 μm, 10 μm, 15 μm, 20 μm, 25 μm, 30 μm, 35 μm, 40 μm, 50 μm, 100 μm, 200 μm, 300 μm, 400 μm, 500 μm, 600 μm, 700 μm, 800 μm, 900 μm, or 1000 m. In some embodiments, the thickness at the site of injection after suprachoroidal administration of the pharmaceutical composition persists for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years.

In some embodiments, the concentration of the transgene in the eye after suprachoroidal administration of the pharmaceutical composition is higher by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%. In some embodiments, the transgene is detected in the eye after suprachoroidal administration of the pharmaceutical composition for at least about 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days. In some embodiments, the transgene is detected in the eye after suprachoroidal administration of the reference pharmaceutical composition for at most about 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, or 100 days, 120 days, 140 days, 160 days, 180 days, 200 days, 220 days, 240 days, 260 days, 280 days, 300 days, 320 days, 340 days, 360 days, 380 days, or 400 days after.

In some embodiments, the longer period of time after suprachoroidal administration of the pharmaceutical composition is longer by at least 4 hours, 8 hours, 12 hours, 16 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

In some embodiments, a level of VEGF-induced vasodilation and/or vascular leakage after suprachoroidal administration of the pharmaceutical composition is equal to or decreased as compared to a level of VEGF-induced vasodilation and/or vascular leakage after suprachoroidal administration of the reference pharmaceutical composition. In some embodiments, the level of VEGF-induced vasodilation and/or vascular leakage after suprachoroidal administration of the pharmaceutical composition is decreased by at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

In some embodiments, the rate of transduction at the site of injection after suprachoroidal administration of the pharmaceutical composition is higher by at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

In some embodiments, the recombinant AAV stability is higher in the pharmaceutical composition as compared to the recombinant AAV stability in the reference pharmaceutical composition. In some embodiments, the recombinant AAV stability is determined by infectivity of the recombinant AAV. In some embodiments, the recombinant AAV stability is determined by a level of aggregation of the recombinant AAV. In some embodiments, the recombinant AAV stability is determined by a level of free DNA released by the recombinant AAV. In some embodiments, the pharmaceutical composition comprises about 50% more, about 25% more, about 15% more, about 10% more, about 5% more, about 4% more, about 3% more, about 2% more, about 1% more, about 0% more, about 1% less, about 2% less, about 5% less, about 7% less, about 10% less, about 2 times more, about 3 times more, about 2 times less, about 3 times less, free DNA as compared to a level of free DNA in the reference pharmaceutical composition.

In some embodiments, the recombinant AAV in the pharmaceutical composition has an infectivity that is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times higher as compared to the infectivity of the recombinant AAV in the reference pharmaceutical composition. In some embodiments, the pharmaceutical composition comprises at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times less recombinant AAV aggregation as compared to a level of the recombinant AAV aggregation in the reference pharmaceutical composition.

In some embodiments, the transgene is a transgene suitable to treat, or otherwise ameliorate, prevent or slow the progression of a disease of interest.

In some embodiments, a human subject is diagnosed with nAMD (wet AMD), dry AMD, retinal vein occlusion (RVO), diabetic macular edema (DME), or diabetic retinopathy (DR), x-linked or Batten disease. In some embodiments, the human subject is diagnosed with glaucoma or non-infectious uveitis. In some embodiments, the human subject is diagnosed with mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis, or kallikrein-related disease.

In some embodiments, the AAV encodes Palmitoyl-Protein Thioesterase 1 (PPT1) or Tripeptidyl-Peptidase 1 (TPP1). In other embodiments, the AAV encodes anti-VEGF fusion protein, anti-VEGF antibody or antigen-binding fragment thereof, anti-kallikrein antibody or antigen-binding fragment, anti-TNF antibody or antigen-binding fragment, anti-C3 antibody or antigen-binding fragment, or anti-C5 antibody or antigen-binding fragment.

In some embodiments, the amount of the recombinant AAV genome copies is based on a vector genome concentration. In some embodiments, the amount of the recombinant AAV genome copies is based on genome copies per administration. In some embodiments, the amount of the recombinant AAV genome copies is based on total genome copies administered to the human subject. In some embodiments, the genome copies per administration is the genome copies of the recombinant AAV per suprachoroidal administration.

In some embodiments, the total genome copies administered is the total genome copies of the recombinant AAV administered suprachoroidally. In some embodiments, the vector genome concentration (VGC) is of about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 6.2×10¹¹ GC/mL, about 1×10¹² GC/mL, about 2.5×10¹² GC/mL, about 3×10¹² GC/mL, about 5×10¹² GC/mL, about 6×10¹² GC/mL, about 1.5×10¹³ GC/mL, about 2×10¹³ GC/mL, or about 3×10¹³ GC/mL. In some embodiments, the total genome copies administered is about 6.0×10¹⁰ genome copies, about 1.6×10¹¹ genome copies, about 2.5×10¹¹ genome copies, about 3.0×10¹¹ genome copies, about 5.0×10¹¹ genome copies, about 6.0×10¹¹ genome copies, about 1.5×10¹² genome copies, about 3×10¹² genome copies, about 1.0×10¹² GC/mL, about 2.5×10¹² GC/mL, or about 3.0×10¹³ genome copies. In some embodiments, the total genome copies administered is about 6.0×10¹⁰ genome copies, about 1.6×10¹¹ genome copies, about 2.5×10¹¹ genome copies, about 5.0×10¹¹ genome copies, about 1.5×10¹² genome copies, about 3×10¹² genome copies, about 1.0×10¹² genome copies, about 2.5×10¹² genome copies, or about 3.0×10¹³ genome copies.

In some embodiments, the pharmaceutical composition is administered once, twice, three times, four times, five times, six times, seven times, eight times, nine times, ten times, fifteen times, twenty times, twenty five times, or thirty times. In some embodiments, the reference pharmaceutical composition is administered once, twice, three times, four times, five times, six times, seven times, eight times, nine times, ten times, fifteen times, twenty times, twenty five times, or thirty times. In some embodiments, the pharmaceutical composition is administered once in one day, twice in one day, three times in one day, four times in one day, five times in one day, six times in one day, or seven times in one day. In some embodiments, the reference pharmaceutical composition is administered once in one day, twice in one day, three times in one day, four times in one day, five times in one day, six times in one day, or seven times in one day.

In some embodiments, the pharmaceutical composition contains poloxamer 407 and poloxamer 188. In some embodiments, the composition comprises 16-22% poloxamer 407. In some embodiments, the composition comprises 0-16% poloxamer 188. In some embodiments, the composition comprises 19% poloxamer 407 and 6% poloxamer 188. In some embodiments, the composition comprises 18% poloxamer 407 and 6.5% poloxamer 188. In some embodiments, the composition comprises 17.5% poloxamer 407 and 7% poloxamer 188

In some embodiments, the composition comprises modified Dulbecco's phosphate-buffered saline solution, and optionally a surfactant. In some embodiments, the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, and optionally a surfactant. In some embodiments, the composition comprises potassium chloride, potassium phosphate monobasic, sodium chloride, sodium phosphate dibasic anhydrous, sucrose, and optionally a surfactant.

In another aspect, provided herein is a method of treating a disease in a subject, the method comprising administering a pharmaceutical composition provided herein to the subject. In some embodiments, the pharmaceutical composition is at a temperature of about 2-10° C. when being administered. In some embodiments, the pharmaceutical composition is at a temperature of about 20-25° C. when being administered.

In some embodiments, the pharmaceutical composition is administered with an injection pressure of less than about 43 PSI. In some embodiments, the pharmaceutical composition is administered with an injection pressure of less than about 65 PSI. In some embodiments, the pharmaceutical composition is administered with an injection pressure of less than about 100 PSI.

In some embodiments, the pharmaceutical composition is administered using a 29 gauge needle. In some embodiments, the pharmaceutical composition is administered using a 30 gauge needle.

In some embodiments, the pharmaceutical composition is administered in an injection time of about 10-15 seconds. In some embodiments, the pharmaceutical composition is administered in an injection time of about 5-30 seconds.

In some embodiments, the subject is human.

In some embodiments, the disease is selected from the group consisting of nAMD (wet AMD), dry AMD, retinal vein occlusion (RVO), diabetic macular edema (DME), diabetic retinopathy (DR), Batten disease, mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis and kallikrein-related disease.

### 3. BRIEF DESCRIPTION OF THE DRAWINGS

FIG. 1. Overview of the localization of Construct II in the suprachoroidal space using a thermoresponsive gel formulation. The thermoresponsive gel formulation is expected to change from liquid state during injection to a gel in the suprachoroidal space and therefore retain the dosed AAV locally in the suprachoroidal space for longer with a greater therapeutic effect.

FIGS. 2A-2C. Calculated injection pressure as a function of viscosity for different 30 gauge and 29 gauge needles. Panel A is scaled to a limit of 100 PSI, panel B to 65 PSI, and panel C to 45 PSI.

FIG. 3. Extraocular temperature measurement using thermal camera.

FIG. 4. Gelation temperature as a function of formulation composition surface plot from design of experiments study.

FIG. 5. Viscosity at 20° C. as a function of formulation composition surface plot from design of experiments study.

FIG. 6. Viscosity at 5° C. as a function of formulation composition surface plot from design of experiments study.

FIG. 7. Summary of gelation temperature rheology data from design of experiment (DOE) study. Samples are labelled with P407-P188 level (e.g. sample #1, labeled “16-0” has 16% P407 and 0% P188).

FIG. 8. Example gelation temperature profile for sample #9 in the DOE study (19% P407/8% P188) showing how the crossover of G′ and G″ is used to determine the gelation temperature.

FIG. 9. Summary of gelation time jump from 20° C. to 34° C. rheology data from DOE Study. Samples are labelled with P407-P188 level (e.g. sample #1 has 16% P407 and 0% P188).

FIG. 10. Summary of gelation time jump from 5° C. to 34° C. rheology data from DOE Study. Samples are labelled with P407-P188 level (e.g. sample #1 has 16% P407 and 0% P188).

FIG. 11. Example gelation time jump from 20° C. to 34° C. profile for sample #9 (19% P407/8% P188) showing how the crossover of G′ and G″ is used to determine the gelation time.

FIG. 12. Summary of viscosity versus shear rate sweep at 20° C. from DOE Study. Samples are labelled with P407-P188 level (e.g. sample #1 has 16% P407 and 0% P188). Note, sample 2 and 4 had already gelled at 20° C., and therefore are showing the impact of shear on breaking the gel structure. All other samples show Newtonian behavior (constant viscosity as function of shear rate).

FIG. 13. Summary of viscosity versus shear rate sweep at 5° C. from DOE Study. Samples are labelled with P407-P188 level (e.g. sample #1 has 16% P407 and 0% P188). All samples show Newtonian behavior (constant viscosity as function of shear rate).

FIG. 14. Thermoresponsive gel formulation design space (white area) with limits of 27 to 32° C. for gel temperature. This design space can represent a scenario where the dose is prepared at 2-8° C. and administered while still chilled or refrigerated. Limits: 15-90 s gel time, viscosity at 5° C.≤183 mPas, injection duration=10 s, and ≥220 μm needle ID). Shaded areas correspond to the regions that exceed the Lo and Hi limits defined for each factor. Contours show the gelation temperature.

FIG. 15. Thermoresponsive gel formulation design space (white area) with limits of 27 to 32° C. for gel temperature (pink shade) and an additional limit on viscosity at 20° C. of ≤183 mPas (region >183 mPas is shown in green shade). In this scenario the dose is administered at controlled room temperature (20° C.) with an injection time of 10 s. Limits: 15-90 s gel time (blue shade), viscosity at 20° C.≤183 mPas (green shade), injection duration=10 s, and ≥220 μm needle ID). Shaded areas correspond to the regions that exceed the Lo and Hi limits defined for each factor. Contours show the gelation temperature. The crosshairs show three formulations further evaluated with: A=6%/19%, B=6.5%/18%, and C=7%17.5% w/v poloxamer 188 and 407 respectively.

FIG. 16. Example preparation of Clinical Drug Product with Autoclave Sterilization.

FIG. 17. Example Preparation of Clinical Drug Product with Sterile Filtration.

FIG. 18. Thermal camera image showing the setup for gelation time for droplet flow on a pre-warmed (31.3° C.) surface (bottle containing warm water for thermal mass). A 50 μL volume of formulation A (left), B (middle) and C (right) at 20° C. were dispensed on the warm surface and a video of the flow of the droplet used to determine the time that the droplet stopped flowing.

FIG. 19. Gelation temperature profile for Formulation A.

FIG. 20. Gelation temperature profile for Formulation B.

FIG. 21. Gelation temperature profile for Formulation C.

FIG. 22. Gelation time jump from 20° C. to 34° C. for Formulation A.

FIG. 23. Gelation time jump from 20° C. to 34° C. for Formulation B.

FIG. 24. Gelation time jump from 20° C. to 34° C. for Formulation C.

FIG. 25. Gelation time jump from 5° C. to 34° C. for Formulation A.

FIG. 26. Gelation time jump from 5° C. to 34° C. for Formulation B.

FIG. 27. Gelation time jump from 5° C. to 34° C. for Formulation C.

FIG. 28. Viscosity versus shear rate at 20° C. for Formulation A.

FIG. 29. Viscosity versus shear rate at 20° C. for Formulation B.

FIG. 30. Viscosity versus shear rate at 20° C. for Formulation C.

FIG. 31. Viscosity versus shear rate at 5° C. for Formulation A.

FIG. 32. Viscosity versus shear rate at 5° C. for Formulation B.

FIG. 33. Viscosity versus shear rate at 5° C. for Formulation C.

FIG. 34. Gelation profile for Formulation A diluted by 10%.

FIG. 35. Gelation profile for Formulation B diluted by 10%.

FIG. 36. Gelation profile for Formulation C diluted by 10%.

FIG. 37. Differential Scanning Fluorometry Profiles of Control (S-0DGN) and Formulations A (S-0DGO), B (S-0DGP), and C (S-0DGQ).

FIG. 38. Injection pressure profile for 0.1 mL of formulation B injected into an enucleated porcine eye at about 35° C. using the CLSD device with 30 Ga needle.

FIG. 39. Injection time profile for 0.1 mL of formulation A, B and C injected into air using a 1 mL syringe with 30 Ga TW needle (pressure/force was held about constant by operator, resulting in longer injection time for higher viscosity formulations).

### 4. DETAILED DESCRIPTION OF THE INVENTION

Provided herein are pharmaceutical compositions comprising recombinant adeno-associated virus (AAV) vector comprising an expression cassette encoding a transgene suitable for administration to a suprachoroidal space (SCS) of an eye of a subject. The subject can be a subject diagnosed with one of more diseases described in Section 4.5. The AAV vectors are described in Section 4.4 and dosages of such vectors are described in Section 4.3. In some embodiments, pharmaceutical compositions provided in Section 4.1 are formulated such that they have one or more functional properties described in Section 4.2. In certain embodiments, the pharmaceutical composition provided herein has various advantages, for example, increased or slower clearance time (Section 4.2.1); decreased circumferential spread (Section 4.2.2); increased SCS thickness (Section 4.2.3); decreased vasodilation and/or vascular leakage (Section 4.2.4); increased AAV level and increased rate of transduction at site of injection (Section 4.2.5); and increased concentration of the transgene after the pharmaceutical composition is administered in the SCS. Without being bound by theory, the functional properties can be achieved using thermoresponsive formulations as disclosed in Section 4.1. Also provided herein are assays that may be used in related studies (Section 4.6).

### 4.1 Formulation of Pharmaceutical Composition

The disclosure provides a pharmaceutical composition suitable for suprachoroidal administration comprising a recombinant adeno-associated virus (AAV) vector comprising an expression cassette encoding a transgene. In some embodiments, several pharmaceutical compositions having different viscosity (or “loss modulus (G″)”) values properties at extraocular temperature (about 32-35° C.) are used to administer an AAV encoding a transgene. In some embodiments, several pharmaceutical compositions having different elastic/storage modulus (G′) properties at extraocular temperature (about 32-35° C.) are used to administer an AAV encoding a transgene.

In some embodiments, the pharmaceutical composition is thermoresponsive. The term “thermoresponsive” is generally known in the art to describe a substance that exhibits different physical properties at different temperatures. In certain embodiments, a pharmaceutical composition provided herein has a lower viscosity, a lower loss modulus (G″), and/or a lower elastic/storage (G′) modulus at room temperature (e.g., about 20-25° C.) than at extraocular temperature (about 32-35° C.). In certain embodiments, a pharmaceutical composition provided herein has a lower viscosity and/or a lower elastic modulus (G′) when chilled (e.g., about 2-10° C.) than at extraocular temperature (about 32-35° C.). A pharmaceutical composition provided herein may be administered to the eye of a subject at a temperature where the viscosity of the pharmaceutical composition is lower (e.g., chilled or at room temperature) that at extraocular temperature (about 32-35° C.). Without wishing to be bound by any particular theory, the change in temperature upon administration to the eye of a subject (e.g., suprachoroidal administration) may lead to an increase in viscosity and/or elastic modulus (G′), resulting in an increased retention time of the composition near the injection site compared to a reference pharmaceutical composition, wherein the reference pharmaceutical composition has a lower viscosity at extraocular temperature (about 32-35° C.).

In some embodiments, the pharmaceutical composition and the reference pharmaceutical composition comprise a recombinant adeno-associated virus (AAV) vector comprising an expression cassette encoding a transgene. In some embodiments, the pharmaceutical composition and the reference pharmaceutical composition have the same vector genome concentration. In some embodiments, the pharmaceutical composition and a reference pharmaceutical composition have the same amount of genome copies. In some embodiments, the pharmaceutical composition at about 32-35° C. has a viscosity and/or elastic modulus (G′) value that is higher than the viscosity of water at about 32-35° C. In some embodiments, the pharmaceutical composition at about 32-35° C. has a viscosity and/or elastic modulus (G′) value that is higher than the viscosity and/or elastic modulus (G′) of a control at about 32-35° C. In some embodiments, the pharmaceutical composition at about 32-35° C. has a viscosity and/or elastic modulus (G′) value that is higher than the viscosity of a solution normally used for subretinal injection at about 32-35° C. In some embodiments, the pharmaceutical composition at about 32-35° C. has a viscosity and/or elastic modulus (G′) value that is higher than the viscosity of PBS or dPBS at about 32-35° C. In some embodiments, the pharmaceutical composition at about 32-35° C. has a viscosity and/or elastic modulus (G′) value that is higher than the viscosity of Hank's Balanced Salt Solution (HBSS) at about 32-35° C. In some embodiments, the reference pharmaceutical composition at about 32-35° C. has lower viscosity and/or elastic modulus (G′) than the pharmaceutical composition at about 32-35° C. In some embodiments, the reference pharmaceutical composition has the same or similar viscosity and/or elastic modulus (G′) as the pharmaceutical composition at about 20-25° C. In some embodiments, the reference pharmaceutical composition is a control solution (e.g., PBS, water, or HBSS). In some embodiments, the reference pharmaceutical composition comprises sucrose. In some embodiments, the reference pharmaceutical composition is a pharmaceutical composition commonly used for AAV subretinal injection. In some embodiments, the reference pharmaceutical composition is not thermoresponsive, e.g., the reference pharmaceutical composition has substantially the same viscosity and/or elastic modulus (G′) at about 20-25° C. as it does at about 32-35° C. or does not have a higher viscosity and/or elastic modulus (G′) at increased temperatures.

In some embodiments, the pharmaceutical composition has viscosity of about, at least about, or at most about 10 cP, 15 cP, 20 cP, 25 cP, 30 cP, 35 cP, 40 cP, 45 cP, 50 cP, 60 cP, 70 cP, 80 cP, 90 cP, 100 cP, 150 cP, 200 cP, 250 cP, 300 cP, 350 cP, 400 cP, 450 cP, 500 cP, 550 cP, 600 cP, 650 cP, 700 cP, 800 cP, 900 cP, 1000 cP, 2,000 cP, 3,000 cP, 4,000 cP, 5,000 cP, 6,000 cP, 7,000 cP, 8,000 c, 9,000 cP, 10,000 cP, 12,000 cP, or 15,000 cP at about 32-35° C., e.g., at zero, 0.001, 0.01, 0.1 or 1 s⁻¹ shear rate or at a shear rate of about or at least about 1000 s⁻¹. In some embodiments, the shear rate is about or less than about 100 s⁻¹, 50 s⁻¹, 10 s⁻¹, 1 s⁻¹, 0.1 s⁻¹, 0.01 s⁻¹, 0.001 s⁻¹, or 0.0001 s⁻¹. In some embodiments, the viscosity of the pharmaceutical composition or the reference pharmaceutical composition is any viscosity disclosed herein at a shear rate of e.g., about or less than about 100 s⁻¹, 50 s⁻¹, 10 s⁻¹, 1 s⁻¹, 0.01 s⁻¹, 0.001 s⁻¹, or 0.0001 s⁻¹.

In some embodiments, the pharmaceutical composition at about 32-35° C. or the reference pharmaceutical composition (or a control pharmaceutical composition or a comparable pharmaceutical composition) at about 32-35° C. has a viscosity (e.g., as measured at a shear rate of about or at least about 1000 s⁻¹) that is about or at least about 5 cP, about or at least about 10 cP, about or at least about 15 cP, about or at least about 20 cP, about or at least about 25 cP, about or at least about 30 cP, about or at least about 35 cP, about or at least about 40 cP, about or at least about 45 cP, about or at least about 50 cP, about or at least about 60 cP, about or at least about 70 cP, about or at least about 80 cP, about or at least about 90 cP, 100 cP, about or at least about 115 cP, about or at least about 120 cP, about or at least about 125 cP, about or at least about 130 cP, about or at least about 135 cP, about or at least about 140 cP, about or at least about 145 cP, about or at least about 150 cP, about or at least about 160 cP, about or at least about 170 cP, about or at least about 180 cP, about or at least about 190 cP, about or at least about 200 cP, about or at least about 300 cP, about or at least about 400 cP, about or at least about 500 cP, about or at least about 600 cP, about or at least about 700 cP, about or at least about 800 cP, about or at least about 900 cP, about or at least about 1000 cP, about or at least about 1500 cP, about or at least about 2000 cP, about or at least about 2500 cP, about or at least about 3000 cP, about or at least about 3500 cP, about or at least about 4000 cP, about or at least about 4500 cP, about or at least about 5000 cP, about or at least about 5500 cP, about or at least about 6000 cP, about or at least about 6500 cP, about or at least about 7000 cP, about or at least about 7500 cP, about or at least about 8000 cP, about or at least about 9000 cP, about or at least about 10000 cP, about or at least about 1×10³ cP, about or at least about 3×10³ cP, about or at least about 1×10⁴ cP, about or at least about 3×10⁴ cP, about or at least about 1×10⁵ cP, about or at least about 1.7×10⁵ cP, about or at least about 3×10⁵ cP, about or at least about 1×10⁶ cP, about or at least about 3×10⁶ cP, about or at least about 1×10⁷ cP, about or at least about 3×10⁷ cP, about or at least about 1×10⁸ cP, about or at least about 3×10⁸ cP. In some embodiments, the viscosity (e.g., as measured at a shear rate of about or at least about 1000 s⁻¹) at about 32-35° C. is between about 25 cP to about 1×10⁶ cP, between about 25 cP to about 1×10⁴ cP, between about 25 cP to about 5,000 cP, between about 25 cP to about 1×10³ cP, between about 100 cP to about 1×10⁶ cP, between about 100 cP to about 1×10⁴ cP, between about 100 cP to about 5,000 cP, between about 100 cP to about 1×10³ cP. In some embodiments, the viscosity (e.g., as measured at a shear rate of about or at least about 1000 s⁻¹) at about 32-35° C. is between about 25 cP to about 3×10⁶ cP, between about 10 cP to about 3×10⁸ cP, between about 50 cP to about 5000 cP, between about 10 cP to about 15000 cP, between about 25 cP to about 1500 cP, between about 50 cP to about 1500 cP, between about 25 cP to about 3×10⁴ cP. In some embodiments, the pharmaceutical composition at about 32-35° C. has a viscosity (e.g., as measured at a shear rate of about or at least about 1000 s⁻¹) that is at least between about 25 cP to about 3×10⁶ cP, at least between about 10 cP to about 3×10⁸ cP, at least between about 50 cP to about 5000 cP, at least between about 10 cP to about 15000 cP, at least between about 25 cP to about 1500 cP, at least between about 50 cP to about 1500 cP, or at least between about 25 cP to about 3×10⁴ cP. In some embodiments, a comparable pharmaceutical composition, or a reference pharmaceutical composition, or a control at about 32-35° C. has a viscosity (e.g., as measured at a shear rate of about or at least about 1000 s⁻¹) of about or at most about 1 cP about or at most about 2 cP, about or at most about 3 cP, about or at most about 4 cP, about or at most about 5 cP, about or at most about 6 cP, about or at most about 7 cP, about or at most about 8 cP, about or at most about 9 cP, about or at most about 10 cP, about or at most about 15 cP, about or at most about 20 cP, about or at most about 25 cP, about or at most about 30 cP, about or at most about 35 cP, about or at most about 40 cP, about or at most about 45 cP, about or at most about 50 cP, about or at most about 55 cP, about or at most about 60 cP, about or at most about 65 cP, about or at most about 70 cP, about or at most about 75 cP, about or at most about 80 cP, about or at most about 85 cP, about or at most about 90 cP, about or at most about 95 cP, about or at most about 100 cP, about or at most about 200 cP, about or at most about 300 cP, about or at most about 400 cP, about or at most about 500 cP. In some embodiments, a comparable pharmaceutical composition, or a reference pharmaceutical composition, or a control at about 32-35° C. has a viscosity of between about 1 cP to about 25 cP, between about 1 cP to about 20 cP, between about 1 cP to about 24 cP, between about 1 cP to about 10 cP, between about 1 cP to about 50 cP, between about 1 cP to about 100 cP, between about 5 cP to about 50 cP, between about 1 cP to about 5 cP, or between about 1 cP to about 200 cP.

In some embodiments, a reference pharmaceutical composition at about 32-35° C. has a viscosity of about 1 cP or less than about 1 cP (e.g., at a shear rate of about or at least about 1000 s⁻¹). In some embodiments, a reference pharmaceutical composition at about 32-35° C. has a viscosity of less than about 1 cP (e.g., at a shear rate of at least about 1000 s⁻¹).

In some embodiments, a pharmaceutical composition provided herein has a viscosity of ≤183 mPas at 20° C. In some embodiments, a pharmaceutical composition provided herein has a viscosity of ≤183 mPas at 5° C. Because viscosity depends on shear rate, the “viscosity” of the pharmaceutical composition is the viscosity at any point between a shear rate of 0.01 s⁻¹ to 100,000 s⁻¹. In some embodiments, the unit for viscosity can be defined as cP or mPas. In some cases, cP and mPas are used interchangeably.

In some embodiments, a pharmaceutical composition provided herein has a viscosity of less than 265 to 655 mPas 32-35° C.

In some embodiments, the viscosity of the pharmaceutical composition at about 32-35° C. is at least about 10 cP or at least about 100 cP or at least about 1000 cP, or at least about 10,000 cP, or at least about 70,000 cP, or up to about 200,000 cP, or up to about 250,000 cP, or up to about 300,000 cP or more. In some embodiments, a shear rate is a shear rate of about or at least about 1000/second. In some embodiments, a formulation is characterized by a zero shear viscosity of at least 300,000 mPas. In some embodiments, the formulation is further characterized by a viscosity of not more than about 400 mPas at 1000 s⁻¹ shear rate.

In some embodiments, a pharmaceutical composition provided herein remains in the SCS (or in the eye) for a longer period of time after injection (measured at different time points) as compared to a reference pharmaceutical formulation, or a formulation having lower viscosity and/or elastic modulus (G′) at about 32-35° C. In some embodiments, a pharmaceutical composition provided herein expands the SCS or the thickness at the site of injection (e.g., as compared to a reference pharmaceutical composition, or formulations having lower viscosity and/or elastic modulus (G′) at about 32-35° C.) (see Section 4.2.3).

In some embodiments, the elastic modulus of a pharmaceutical composition provided herein at under 27° C. is less than about or about 0.1 Pa, less than about or about 0.01 Pa, less than about or about 0.001 Pa or zero. In some embodiments, the elastic modulus of a pharmaceutical composition provided herein at 32° C. to 35° C. is about or at least about 0.1 Pa, about or at least about 1 Pa, about or at least about 10 Pa, about or at least about 100 Pa, about or at least about 1000 Pa, about or at least about 10,000 Pa or about or at least about 100,000 Pa.

In some embodiments, a pharmaceutical composition provided herein has a gelation temperature of over 27° C. In some embodiments, a pharmaceutical composition provided herein has a gelation temperature of less than 32° C. In some embodiments, a pharmaceutical composition provided herein has a gelation temperature over about 27-32° C. In some embodiments, a pharmaceutical composition provided herein has a gelation temperature of about 25, 26, 27, 28, 29, 30, 31, 32, 33, 34, or 35° C.

In some embodiments, a pharmaceutical composition provided herein has a gelation time of longer than about 10 seconds. In some embodiments, a pharmaceutical composition provided herein has a gelation time of longer than about 15 seconds. In some embodiments, a pharmaceutical composition provided herein has a gelation time of about 10-15 seconds, about 15-20 seconds, about 20-25 seconds, about 25-30 seconds, about 30-35 seconds, about 35-40 seconds, about 40-45 seconds, about 45-50 seconds, about 50-55 seconds, about 55-60 seconds, about 60-65 seconds, about 65-70 seconds, about 70-75 seconds, about 75-80 seconds, about 80-85 seconds, or about 85-90 seconds. In some embodiments, a pharmaceutical composition provided herein is a gelation time of less than 90 seconds. In some embodiments, the gelation time is determined at about 34° C. In some embodiments, the gelation time is determined at about 32-34° C. In some embodiments, the gelation time of a pharmaceutical composition is longer than the injection time of said composition. In some embodiments, the gelation time is 10%, 20%, 30%, 40%, 50%, 60%, 70%, 80%, 90% or more than 90% longer than the injection time.

In some embodiments, the pharmaceutical composition at about 32-35° C. has a viscosity sufficient to expand at least a portion of the site of injection (e.g. SCS) to a thickness of at least 500 μm or about 500 μm to about 3 mm, for at least two hours after administration. In some embodiments, the viscosity of the pharmaceutical composition at about 32-35° C. is sufficient to expand the site of injection (e.g. SCS) to a thickness of about 750 μm to about 2.8 mm, about 750 μm to about 2.5 mm, about 750 μm to about 2 mm, or about 1 mm to about 2 mm. In some embodiments, the viscosity of the pharmaceutical composition at about 32-35° C. is sufficient to expand the site of injection (e.g. SCS) to a thickness of about 500 μm to about 3.0 mm for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years after the administration. In some embodiments, the viscosity of the pharmaceutical composition at about 32-35° C. is sufficient to expand the site of injection (e.g. SCS) to a thickness of about 1 mm to about 3 mm for at least two hours, at least three hours, at least four hours, at least five hours, at least six hours, at least seven hours, at least eight hours, at least ten hours, at least twelve hours, at least eighteen hours, or at least twenty-four hours after administration. In some embodiments, the viscosity of the pharmaceutical composition at about 32-35° C. is sufficient to expand the site of injection (e.g. SCS) to a thickness of about 1 mm to about 2 mm for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years after the administration. In some embodiments, the viscosity of the pharmaceutical composition at about 32-35° C. is sufficient to expand the site of injection (e.g. SCS) to a thickness of about 2 mm to about 3 mm for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years after the administration. In some embodiments, the viscosity of the pharmaceutical composition at about 32-35° C. is sufficient to expand the site of injection (e.g. SCS) to a thickness of about 750 μm to about 2.8 mm, about 750 μm to about 2.5 mm, about 750 m to about 2 mm, or about 1 mm to about 2 mm for an indefinite period. An indefinite period may be achieved due, at least in part, to the stability of the pharmaceutical composition in the site of injection (e.g. SCS).

In some embodiments, a pharmaceutical composition at about 32-35° C. having a viscosity sufficient to expand the site of injection (e.g. SCS) to a thickness of at least 500 μm, or about 500 μm to about 3 mm, has a viscosity greater than the viscosity of water at about 32-35° C. (i.e., about 1 cP). In some embodiments, a pharmaceutical composition at about 32-35° C. has a viscosity sufficient to expand the site of injection (e.g. SCS) to a thickness of at least about 50 μm, 100 μm, 200 μm, 300 μm, 400 μm, 500 μm, 600 μm, 700 μm, 800 μm, 900 μm, 1000 μm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, or larger than 10 mm. In some embodiments, a reference pharmaceutical composition at about 32-35° C. has a viscosity sufficient to expand the site of injection to a thickness of at most about 1 nm, 5 nm, 10 nm, 25 nm, 50 nm, 100 nm, 200 nm, 300 nm, 400 nm, 500 nm, 600 nm, 700 nm, 800 nm, 900 nm, 1 μm, 5 μm, 10 μm, 15 μm, 20 μm, 25 μm, 30 μm, 35 μm, 40 μm, 50 μm, 100 μm, 200 μm, 300 μm, 400 μm, 500 μm, 600 μm, 700 μm, 800 μm, 900 μm, 1000 μm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm.

Also provided herein are methods of treating a disease (e.g., an ocular disease) described in Section 4.5 using the pharmaceutical compositions disclosed herein. In some embodiments, a method of treating an ocular disease includes administering an effective amount of the pharmaceutical composition (e.g., recombinant adeno-associated virus (AAV) vector comprising an expression cassette encoding a transgene) to a subject (e.g., human). In some embodiments, the pharmaceutical composition is administered in the suprachoroidal space (SCS) of an eye of the subject. In some embodiments, the effective amount of the pharmaceutical composition sufficient to elicit a therapeutic response when administered to the SCS is less than the effective amount of the pharmaceutical composition sufficient to elicit a therapeutic response when administered subretinally. In some embodiments, the effective amount of the pharmaceutical composition sufficient to elicit a therapeutic response when administered to the SCS is less than the effective amount of the pharmaceutical composition sufficient to elicit a therapeutic response when administered intravitreously. In some embodiments, the pharmaceutical composition has the same vector genome concentration when administered to the SCS as when administered via subretinal administration or via intravitreous administration. In some embodiments, the pharmaceutical composition has the same amount of genome copies when administered to the SCS as when administered via subretinal administration or via intravitreous administration. In some embodiments, the effective amount of the pharmaceutical composition sufficient to elicit a therapeutic response in a subject is lower as compared to the effective amount of a reference pharmaceutical composition sufficient to elicit a therapeutic response in the subject when administered to the SCS. In some embodiments, the effective amount of the pharmaceutical composition sufficient to elicit a therapeutic response when administered to the SCS is less than the effective amount of a reference pharmaceutical composition sufficient to elicit a therapeutic response when administered subretinally. In some embodiments, the effective amount of the pharmaceutical composition sufficient to elicit a therapeutic response when administered to the SCS is less than the effective amount of a reference pharmaceutical composition sufficient to elicit a therapeutic response when administered intravitreously. In some embodiments, the pharmaceutical composition and the reference pharmaceutical composition have the same vector genome concentration. In some embodiments, the pharmaceutical composition and the reference pharmaceutical composition have the same amount of genome copies. In some embodiments, the pharmaceutical composition has a viscosity and/or elastic modulus (G′) that is higher than the viscosity and/or elastic modulus (G′) of the reference pharmaceutical composition.

In some embodiments, the pharmaceutical composition is substantially localized near the insertion site (see Section 4.2.1 and Section 4.2.2). In some embodiments, the pharmaceutical composition results in a higher level of transgene expression (concentration) when the pharmaceutical composition is administered in the SCS as compared to when the pharmaceutical composition is administered subretinally or intravitreously (see Section 4.2.6). In some embodiments, the pharmaceutical composition results in a higher level of transgene expression (concentration) when the pharmaceutical composition is administered in the SCS as compared to when a reference pharmaceutical composition is administered subretinally, intravitreously, or in the SCS (see Section 4.2.6). In some embodiments, the pharmaceutical composition results in a higher level of AAV when the pharmaceutical composition is administered in the SCS as compared to when the pharmaceutical composition is administered subretinally or intravitreously (see Section 4.2.5). In some embodiments, the pharmaceutical composition results in a higher level of AAV when the pharmaceutical composition is administered in the SCS as compared to when a reference pharmaceutical composition is administered subretinally, intravitreously, or in the SCS (see Section 4.2.5). In some embodiments, the pharmaceutical composition results in a higher rate of transduction (or rate of infection) at a site of injection when the pharmaceutical composition is administered in the SCS as compared to when the pharmaceutical composition is administered subretinally or intravitreously (see Section 4.2.5). In some embodiments, the pharmaceutical composition results in a higher rate of transduction (or rate of infection) at a site of injection when the pharmaceutical composition is administered in the SCS as compared to when a reference pharmaceutical composition is administered subretinally, intravitreously, or in the SCS (see Section 4.2.5). In some embodiments, the pharmaceutical composition results in reduced vasodilation and/or vascular leakage when the pharmaceutical composition is administered in the SCS as compared to when the pharmaceutical composition is administered subretinally or intravitreously (see Section 4.2.4). In some embodiments, the pharmaceutical composition results in reduced vasodilation and/or vascular leakage when the pharmaceutical composition is administered in the SCS as compared to when a reference pharmaceutical composition is administered subretinally, intravitreously, or in the SCS (see Section 4.2.4). In some embodiments, the reference pharmaceutical composition includes the recombinant adeno-associated virus (AAV) vector comprising the expression cassette encoding the transgene. In some embodiments, the pharmaceutical composition at about 32-35° C. has higher viscosity and/or elastic modulus (G′) than the reference pharmaceutical composition at about 32-35° C. In some embodiments, the pharmaceutical composition and the reference pharmaceutical composition have the same vector genome concentration. In some embodiments, the pharmaceutical composition and the reference pharmaceutical composition have the same amount of genome copies.

### 4.1.1 Manipulation of Viscosity

In some embodiments, the viscosity and/or elastic modulus (G′) of a pharmaceutical composition provided herein increase to values well in excess of the viscosity of water (for example, at least about 100 cP at a shear rate of 0.1/second) as the formulation warms to at about 32-35° C., resulting in formulations that are highly effective for placement, e.g., injection, into an eye of a subject (e.g., to the SCS). In some embodiments, the relatively high viscosity and/or elastic modulus of the formulation at about 32-35° C. enhances the ability of such formulations to maintain the therapeutic component (e.g., AAV comprising an expression cassette comprising a transgene) in substantially uniform suspension in the formulation for prolonged periods of time, and can also aid in the storage stability of the formulation.

A pharmaceutical composition provided herein (e.g., a thermoresponsive pharmaceutical composition) may comprise a liquid dispersion medium (the “solvent”) and the gelling agent (the “gelator”). The solvent molecules may penetrate a hydrocolloidal network formed by the gelator. In some embodiments, a pharmaceutical composition provided herein comprises hydrophilic polymers in an aqueous system. In some embodiments, a pharmaceutical composition provided herein comprises natural polymers (e.g., xanthan gum, starch, gellan, konjac, carrageenans, collagen, fibrin, silk fibroin, hyaluronic acid or gelatin). In some embodiments, a pharmaceutical composition provided herein comprises synthetic polymers (e.g., chitosan-β-glycerophosphate, poly (N-Isopropylacrylamide) (pNIPAAm), pluronic F127, methylcellulose or PEG-PCL). See, e.g., Taylor et al., Gels. 2017 March; 3(1): 4.

Non-limiting examples of solutions that have a higher viscosity and/or elastic modulus (G′) at about 32-35° C. compared to lower temperatures and that can be used in a pharmaceutical composition of the present disclosure include solutions comprising varying concentrations of poloxamer 407 (P407, CAS Number: 9003-11-6) and poloxamer 188 (P188, CAS Number: 9003-11-6). In some embodiments, a pharmaceutical composition provided herein comprises 16% P407 and 0% P188. In some embodiments, a pharmaceutical composition provided herein comprises 22% P407 and 0% P188. In some embodiments, a pharmaceutical composition provided herein comprises 16% P407 and 16% P188. In some embodiments, a pharmaceutical composition provided herein comprises 22% P407 and 16% P188. In some embodiments, a pharmaceutical composition provided herein comprises 19% P407 and 0% P188. In some embodiments, a pharmaceutical composition provided herein comprises 16% P407 and 8% P188. In some embodiments, a pharmaceutical composition provided herein comprises 22% P407 and 8% P188. In some embodiments, a pharmaceutical composition provided herein comprises 19% P407 and 8% P188.

### 4.1.2 Other Components of the Formulation

In some embodiments, the disclosure provides a pharmaceutical composition (e.g., liquid formulation) comprising a recombinant adeno-associated virus (AAV) and at least one of: potassium phosphate monobasic, sodium chloride, sodium phosphate dibasic anhydrous, sucrose, and surfactant. In some embodiments, the pharmaceutical composition (e.g., liquid formulation) does not comprise sucrose. Assays such as those described in Section 4.6 and/or Section 5 can be used to determine that the presence of additional components does not interfere with the properties of the present formulations such as higher viscosity and/or elastic modulus (G′) at increased temperatures.

In some embodiments, the disclosure provides a pharmaceutical composition comprising a recombinant adeno-associated virus (AAV) and at least one of: an ionic salt excipient or buffering agent, sucrose, and surfactant. In some embodiments, the ionic salt excipient or buffering agent can be one or more components from the group consisting of potassium phosphate monobasic, potassium phosphate, sodium chloride, sodium phosphate dibasic anhydrous, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, and calcium citrate. In some embodiments, the surfactant can be one or more components from the group consisting of poloxamer 188, polysorbate 20, and polysorbate 80.

In certain embodiments, the pharmaceutical composition has an ionic strength of about 60 mM to about 115 mM. In certain embodiments, the pharmaceutical composition has an ionic strength of about 60 mM to about 100 mM. In certain embodiments, the pharmaceutical composition has an ionic strength of about 65 mM to about 95 mM. In certain embodiments, the pharmaceutical composition has an ionic strength of about 70 mM to about 90 mM. In certain embodiments, the pharmaceutical composition has an ionic strength of about 75 mM to about 85 mM.

In certain embodiments, the pharmaceutical composition has an ionic strength of about 30 mM to about 100 mM. In certain embodiments, the pharmaceutical composition has an ionic strength of about 35 mM to about 95 mM. In certain embodiments, the pharmaceutical composition has an ionic strength of about 40 mM to about 90 mM. In certain embodiments, the pharmaceutical composition has an ionic strength of about 45 mM to about 85 mM. In certain embodiments, the pharmaceutical composition has an ionic strength of about 50 mM to about 80 mM. In certain embodiments, the pharmaceutical composition has an ionic strength of about 55 mM to about 75 mM. In certain embodiments, the pharmaceutical composition has an ionic strength of about 60 mM to about 70 mM.

In certain embodiments, the pharmaceutical composition comprises potassium chloride (e.g., at a concentration of 0.2 g/L). In certain embodiments, the pharmaceutical composition comprises potassium phosphate monobasic (e.g., at a concentration of 0.2 g/L). In certain embodiments, the pharmaceutical composition comprises sodium chloride (e.g., at a concentration of 5.84 g/L). In certain embodiments, the pharmaceutical composition comprises sodium phosphate dibasic anhydrous (e.g., at a concentration of 1.15 g/L). In certain embodiments, the pharmaceutical composition comprises potassium chloride, potassium phosphate monobasic, sodium chloride, and sodium phosphate dibasic anhydrous.

In certain embodiments, the pharmaceutical composition comprises sucrose at a concentration of 3% (weight/volume, 30 g/L) to 18% (weight/volume, 180 g/L). In certain embodiments, the pharmaceutical composition comprises sucrose at a concentration of 4% (weight/volume, 40 g/L).

In certain embodiments, the pharmaceutical composition comprises poloxamer 188 at a concentration of 0.001% (weight/volume, 0.01 g/L). In certain embodiments, the pharmaceutical composition comprises poloxamer 188 at a concentration of 0.0005% (weight/volume, 0.005 g/L) to 0.05% (weight/volume, 0.5 g/L). In certain embodiments, the pharmaceutical composition comprises poloxamer 188 at a concentration of 0.001% (weight/volume, 0.01 g/L). In certain embodiments, the pharmaceutical composition comprises polysorbate 20 at a concentration of 0.0005% (weight/volume, 0.05 g/L) to 0.05% (weight/volume, 0.5 g/L). In certain embodiments, the pharmaceutical composition comprises polysorbate 80 at a concentration of 0.0005% (weight/volume, 0.05 g/L) to 0.05% (weight/volume, 0.5 g/L).

In certain embodiments, the pH of the pharmaceutical composition is about 7.4. In certain embodiments, the pH of the pharmaceutical composition is about 6.0 to 9.0. In certain embodiments, the pH of the pharmaceutical composition is 7.4. In certain embodiments, the pH of the pharmaceutical composition is 6.0 to 9.0.

In certain embodiments, the pharmaceutical composition is in a hydrophobically-coated glass vial. In certain embodiments, the pharmaceutical composition is in a Cyclo Olefin Polymer (COP) vial. In certain embodiments, the pharmaceutical composition is in a Daikyo Crystal Zenith® (CZ) vial. In certain embodiments, the pharmaceutical composition is in a TopLyo coated vial.

In certain embodiments, disclosed herein is a pharmaceutical composition comprising a recombinant AAV and at least one of: (a) potassium chloride at a concentration of 0.2 g/L, (b) potassium phosphate monobasic at a concentration of 0.2 g/L, (c) sodium chloride at a concentration of 5.84 g/L, (d) sodium phosphate dibasic anhydrous at a concentration of 1.15 g/L, (e) sucrose at a concentration of 4% weight/volume (40 g/L), (f) poloxamer 188 at a concentration of 0.001% weight/volume (0.01 g/L), and (g) water, and wherein the recombinant AAV is AAV8. In some embodiments, the pharmaceutical composition does not comprise sucrose.

In some embodiments, the pharmaceutical composition comprises (a) the Construct II encoding an anti-human vascular endothelial growth factor (hVEGF) antibody and at least one of: (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the anti-hVEGF antibody comprises a heavy chain comprising the amino acid sequence of SEQ ID NO:2 or SEQ ID NO:4, and a light chain comprising the amino acid sequence of SEQ ID NO:1 or SEQ ID NO:3. In some embodiments, the pharmaceutical composition does not comprise sucrose.

In some embodiments, the pharmaceutical composition comprises (a) an AAV8 or AAV9 that encodes Tripeptidyl-Peptidase 1 and at least one of: (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water. In some embodiments, the pharmaceutical composition does not comprise sucrose.

In some embodiments, the pharmaceutical composition has desired viscosity, elastic modulus (G′), density, and/or osmolality that is suitable for suprachoroidal injection (for example, via a suprachoroidal drug delivery device such as a microinjector with a microneedle). In some embodiments, the pharmaceutical composition is a liquid composition. In some embodiments, the pharmaceutical composition is a frozen composition. In some embodiments, the pharmaceutical composition is a gel.

In certain embodiments, the pharmaceutical composition has a osmolality range of 200 mOsm/L to 660 mOsm/L. In certain embodiments, the pharmaceutical composition has a osmolality of about, of at least about, or of at most about: 200 mOsm/L, 250 mOsm/L, 300 mOsm/L, 350 mOsm/L, 400 mOsm/L, 450 mOsm/L, 500 mOsm/L, 550 mOsm/L, 600 mOsm/L, 650 mOsm/L, or 660 mOsm/L.

In certain embodiments, gene therapy constructs are supplied as a frozen sterile, single use solution of the AAV vector active ingredient in a formulation buffer. In a specific embodiment, the pharmaceutical compositions suitable for suprachoroidal administration comprise a suspension of the recombinant (e.g., rHuGlyFabVEGFi) vector in a formulation buffer comprising a physiologically compatible aqueous buffer, a surfactant and optional excipients. In some embodiments, the construct is formulated in Dulbecco's phosphate buffered saline and 0.001% poloxamer 188, pH=7.4.

In specific embodiments, the composition comprises modified Dulbecco's phosphate-buffered saline solution, and optionally a surfactant. In other specific embodiments, the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, and optionally a surfactant. In other specific embodiments, the composition comprises potassium chloride, potassium phosphate monobasic, sodium chloride, sodium phosphate dibasic anhydrous, sucrose, and optionally a surfactant.

In some embodiments, a pharmaceutical composition provided herein is not a composition described in Zeinab et al. (European Journal of Pharmaceutics and Biopharmaceutics 114 (2017) 119-13).

### 4.2 Functional Properties

### 4.2.1 Clearance Time

The disclosure provides a pharmaceutical composition (e.g., a composition comprising an AAV comprising an expression cassette encoding a transgene) resulting in a delayed clearance time from the SCS. In some embodiments, a pharmaceutical composition that is viscous (or more viscous) and/or elastic and/or gelled (or more elastic and/or more gelled) at about 32-35° C. results in delayed clearance time from the SCS as compared to a pharmaceutical composition which is non-viscous or low viscosity and/or less elastic and/or is not gelled at about 32-35° C. In some embodiments, a pharmaceutical composition that is viscous (or more viscous) and/or elastic and/or gelled (or more elastic and/or more gelled) at about 32-35° C. results in delayed clearance time from the eye as compared to a pharmaceutical composition which is non-viscous or low viscosity and/or less elastic and/or is not gelled at about 32-35° C. In some embodiments, a more viscous and/or elastic and/or gelled pharmaceutical composition results in delayed clearance time from the eye as compared to a less viscous and/or elastic and/or gelled pharmaceutical composition. In some embodiments, a pharmaceutical composition that is more viscous and/or elastic and/or gelled at about 32-35° C. has a viscosity value that is higher than the viscosity of water at about 32-35° C. In some embodiments, a pharmaceutical composition that is more viscous and gelled at about 32-35° C. has a viscosity value and/or an elastic modulus value that is higher than the viscosity and/or elastic modulus of a solution normally used for subretinal injection at about 32-35° C. In some embodiments, the clearance time of the pharmaceutical composition after the pharmaceutical composition is administered to the SCS is equal to or higher than the clearance time of a reference pharmaceutical composition after the reference pharmaceutical composition is administered subretinally or intravitreously. In some embodiments, the clearance time of the pharmaceutical composition after the pharmaceutical composition is administered to the SCS is equal to or higher than the clearance time of a reference pharmaceutical composition after the reference pharmaceutical composition is administered to the SCS.

In some embodiments, a pharmaceutical composition (e.g., a composition comprising an AAV comprising an expression cassette encoding a transgene) results in a clearance time from the SCS of about 30 minutes to about 20 hours, about 2 hours to about 20 hours, about 30 minutes to about 24 hours, about 1 hour to about 2 hours, about 30 minutes to about 90 days, about 30 minutes to about 60 days, about 30 minutes to about 30 days, about 30 minutes to about 21 days, about 30 minutes to about 14 days, about 30 minutes to about 7 days, about 30 minutes to about 3 days, about 30 minutes to about 2 days, about 30 minutes to about 1 day, about 4 hours to about 90 days, about 4 hours to about 60 days, about 4 hours to about 30 days, about 4 hours to about 21 days, about 4 hours to about 14 days, about 4 hours to about 7 days, about 4 hours to about 3 days, about 4 hours to about 2 days, about 4 hours to about 1 day, about 4 hours to about 8 hours, about 4 hours to about 16 hours, about 4 hours to about 20 hours, about, 1 day to about 90 days, about 1 day to about 60 days, about 1 day to about 30 days, about 1 day to about 21 days, about 1 day to about 14 days, about 1 day to about 7 days, about 1 day to about 3 days, about 2 days to about 90 days, about 3 days to about 90 days, about 3 days to about 60 days, about 3 days to about 30 days, about 3 days to about 21 days, about 3 days to about 14 days, or about 3 days to about 7 days. In some embodiments, the clearance time from the SCS is of about 3 days to about 365 days, about 3 days to about 300 days, about 3 days to about 200 days, about 3 days to about 150 days, about 3 days to about 125 days, about 7 days to about 365 days, about 7 days to about 300 days, about 7 days to about 200 days, about 7 days to about 150 days, about 7 days to about 125 days. The “clearance time from the SCS” is the time required for substantially all of the pharmaceutical composition, the pharmaceutical agent, or the AAV to escape the SCS. In some embodiments, the “clearance time from the SCS” is the time required for the pharmaceutical composition, the pharmaceutical agent, or the AAV to not be detected in the SCS by any standard method (such as those described in Section 4.6 and Section 5). In some embodiments, the “clearance time from the SCS” is when the pharmaceutical composition, the pharmaceutical agent, or the AAV is present in the SCS in an amount that is at most about 2% or at most about 5% as detected by any standard method (such as those described in Section 4.6 and Section 5).

In some embodiments, the pharmaceutical composition (e.g., a composition comprising an AAV comprising an expression cassette encoding a transgene) results in a clearance time from the eye of about 30 minutes to about 20 hours, about 2 hours to about 20 hours, about 30 minutes to about 24 hours, about 1 hour to about 2 hours, about 30 minutes to about 90 days, about 30 minutes to about 60 days, about 30 minutes to about 30 days, about 30 minutes to about 21 days, about 30 minutes to about 14 days, about 30 minutes to about 7 days, about 30 minutes to about 3 days, about 30 minutes to about 2 days, about 30 minutes to about 1 day, about 4 hours to about 90 days, about 4 hours to about 60 days, about 4 hours to about 30 days, about 4 hours to about 21 days, about 4 hours to about 14 days, about 4 hours to about 7 days, about 4 hours to about 3 days, about 4 hours to about 2 days, about 4 hours to about 1 day, about 4 hours to about 8 hours, about 4 hours to about 16 hours, about 4 hours to about 20 hours, about 1 day to about 90 days, about 1 day to about 60 days, about 1 day to about 30 days, about 1 day to about 21 days, about 1 day to about 14 days, about 1 day to about 7 days, about 1 day to about 3 days, about 2 days to about 90 days, about 3 days to about 90 days, about 3 days to about 60 days, about 3 days to about 30 days, about 3 days to about 21 days, about 3 days to about 14 days, or about 3 days to about 7 days. In some embodiments, the clearance time from the eye is of about 3 days to about 365 days, about 3 days to about 300 days, about 3 days to about 200 days, about 3 days to about 150 days, about 3 days to about 125 days, about 7 days to about 365 days, about 7 days to about 300 days, about 7 days to about 200 days, about 7 days to about 150 days, about 7 days to about 125 days. The “clearance time from the eye” is the time required for substantially all of the pharmaceutical composition, the pharmaceutical agent, or the AAV to escape the eye. In some embodiments, the “clearance time from the eye” is the time required for the pharmaceutical composition, the pharmaceutical agent, or the AAV to not be detected in the eye by any method (such as those described in Section 4.6 and Section 5). In some embodiments, the “clearance time from the eye” is when the pharmaceutical composition, the pharmaceutical agent, or the AAV is present in the eye in an amount that is at most about 2% or at most about 5% as detected by any standard method (such as those described in Section 4.6 and Section 5).

In some embodiments, the clearance time is not prior to (e.g., the clearance time from the SCS or the eye does not occur before) about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after administration of the pharmaceutical composition. In some embodiments, the clearance time is about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after administration of the pharmaceutical composition.

In some embodiments, a pharmaceutical composition (e.g., a composition comprising an AAV comprising an expression cassette encoding a transgene) that is more viscous and/or more elastic and/or gelled at about 32-35° C. results in a clearance time that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a less viscous and/or less elastic and/or non-gelled pharmaceutical composition is used to administer the AAV comprising the expression cassette encoding the transgene (e.g., via a subretinal administration, intravitreous administration, or to the SCS).

In some embodiments, a suprachoroidal administration of a pharmaceutical composition (e.g., a composition comprising an AAV comprising an expression cassette encoding a transgene) that is more viscous and/or more elastic and/or gelled at about 32-35° C. results in a clearance time that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a pharmaceutical composition which is less viscous and/or less elastic and/or non-gelled at about 32-35° C. is used, for example, to administer the AAV comprising the expression cassette encoding the transgene by suprachoroidal administration.

In some embodiments, a suprachoroidal administration of a pharmaceutical composition (e.g., a composition comprising an AAV comprising an expression cassette encoding a transgene) that is more viscous and/or more elastic and/or gelled at about 32-35° C. results in a clearance time that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a pharmaceutical composition which is less viscous at about 32-35° C. is used, for example, to administer the AAV comprising the expression cassette encoding the transgene by subretinal administration or by intravitreous administration.

In some embodiments, a suprachoroidal administration of a pharmaceutical composition which is viscous (e.g., relatively viscous, medium to super high viscosity, or more viscous than water, or more viscous than a control solution, or more viscous than a solution commonly used for subretinal administration) at about 32-35° C. (e.g., a composition comprising an AAV comprising an expression cassette encoding a transgene) results in a clearance time that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when the same pharmaceutical composition is used, for example, to administer the AAV comprising the expression cassette encoding the transgene via subretinal administration or via intravitreous administration.

In some embodiments, the clearance time of a pharmaceutical composition (e.g., a pharmaceutical composition comprising an AAV comprising an expression cassette encoding a transgene) that is relatively viscous and/or elastic and/or gelled at about 32-35° C. administered by suprachoroidal injection is greater than the clearance time of the same pharmaceutical composition administered via subretinal administration or via intravitreous administration. In some embodiments, the clearance time of a pharmaceutical composition (e.g., a pharmaceutical composition comprising an AAV comprising an expression cassette encoding a transgene) that is more viscous and/or more elastic and/or gelled at about 32-35° C. administered by suprachoroidal injection is greater than a pharmaceutical composition which is comparably less viscous and/or less elastic and/or non-gelled at about 32-35° C. administered by suprachoroidal injection. In some embodiments, the clearance time of a pharmaceutical composition (e.g., a pharmaceutical composition comprising an AAV comprising an expression cassette encoding a transgene) that is more viscous and/or more elastic and/or gelled at about 32-35° C. administered by suprachoroidal injection is greater than a pharmaceutical composition which is comparable less viscous and/or less elastic and/or non-gelled at about 32-35° C. administered via subretinal administration or via intravitreous administration. In some embodiments, the clearance time of a pharmaceutical composition (e.g., a pharmaceutical composition comprising an AAV comprising an expression cassette encoding a transgene) that is viscous and/or elastic and/or gelled at about 32-35° C. administered by suprachoroidal injection is greater than a pharmaceutical composition that is comparably viscous and/or elastic and/or gelled at about 32-35° C. administered via subretinal administration or via intravitreous administration.

In some embodiments, the clearance time of a pharmaceutical composition (e.g., a pharmaceutical composition comprising an AAV comprising an expression cassette encoding a transgene) that is viscous and/or elastic and/or gelled at about 32-35° C. administered by suprachoroidal injection is greater than the same pharmaceutical composition administered via subretinal administration or via intravitreous administration by at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

In some embodiments, the clearance time of a pharmaceutical composition (e.g., a pharmaceutical composition comprising an AAV comprising an expression cassette encoding a transgene) which is more viscous and/or more elastic and/or gelled at about 32-35° C. administered by suprachoroidal injection is greater than a pharmaceutical composition which is comparably less viscous and/or less elastic and/or not gelled at about 32-35° C. administered by suprachoroidal injection by at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

In some embodiments, the clearance time of a pharmaceutical composition (e.g., a pharmaceutical composition comprising an AAV comprising an expression cassette encoding a transgene) which is more viscous and/or more elastic and/or gelled at about 32-35° C. administered by suprachoroidal injection is greater than a pharmaceutical composition that is comparably less viscous and/or less elastic and/or not gelled at about 32-35° C. administered via subretinal administration or via intravitreous administration by at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

In some embodiments, the clearance time of the pharmaceutical composition administered via intravitreous injection or via subretinal injection is of at most about 30 minutes, 1 hours, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or at most about 400 days after administration.

In some embodiments, the clearance time of a reference pharmaceutical composition administered by intravitreous injection, subretinal injection, or to the SCS is of at most about 30 minutes, 1 hours, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or at most about 400 days after administration.

In some embodiments, the clearance time is the clearance time from the eye. In some embodiments, the clearance time is the clearance time from the SCS. In some embodiments, the clearance time is the clearance time from the site of injection.

### 4.2.2 Circumferential Spread

In some embodiments, a pharmaceutical composition localizes at the site of injection. In some embodiments, a pharmaceutical composition localizes at the site of injection for a longer period of time than a comparable pharmaceutical composition which has a lower viscosity and/or elastic modulus (G′) and/or is not gelled at extraocular temperature (about 32-35° C.). In some embodiments, a pharmaceutical composition localizes at the site of injection for a longer period of time when injected in the SCS as compared to when the pharmaceutical composition is administered by subretinal injection or intravitreous injection. The pharmaceutical composition can have different viscosity and/or elastic modulus values. In some embodiments, a pharmaceutical composition that is viscous and/or elastic and/or gelled (or more viscous, more elastic and/or more gelled) at about 32-35° C. remains localized in the SCS for a longer period of time as compared to a pharmaceutical composition that is a non-viscous or has low viscosity and/or is not gelled at about 32-35° C.

In some embodiments, localization can be determined by evaluating circumferential spread (e.g., 2D circumferential spread). In some embodiments, a pharmaceutical composition (e.g., a composition comprising an AAV comprising an expression cassette encoding a transgene) results in a circumferential spread that is at least 2 times less, at least 3 times less, at least 4 times less, at least 5 times less, at least 6 times less, at least 7 times less, at least 8 times less, at least 9 times less, at least 10 times less, at least 15 times less, at least 20 times less, at least 50 times less, at least 100 times less, at least 5% less, at least 10% less, at least 15% less, at least 20% less, at least 25% less, at least 30% less, at least 35% less, at least 40%, at least 45% less, at least 50% less, at least 55% less, at least 60% less, at least 65% less, at least 70% less, at least 75% less, at least 80% less, at least 85% less, at least 90% less, at least 95% less, at least 100% less, at least 150% less, or at least 200% less, at least 250% less, or at least 300%, at least 400% less, or at least 500% less than when a reference pharmaceutical composition is used to administer the AAV comprising the expression cassette encoding the transgene (e.g., by suprachoroidal injection, by subretinal injection, or by intravitreous injection), which is less viscous at extraocular temperature (about 32-35° C.).

In some embodiments, a suprachoroidal administration of a pharmaceutical composition (e.g., a composition comprising an AAV comprising an expression cassette encoding a transgene) results in a circumferential spread that is at least 2 times less, at least 3 times less, at least 4 times less, at least 5 times less, at least 6 times less, at least 7 times less, at least 8 times less, at least 9 times less, at least 10 times less, at least 15 times less, at least 20 times less, at least 50 times less, at least 100 times less, at least 5% less, at least 10% less, at least 15% less, at least 20% less, at least 25% less, at least 30% less, at least 35% less, at least 40%, at least 45% less, at least 50% less, at least 55% less, at least 60% less, at least 65% less, at least 70% less, at least 75% less, at least 80% less, at least 85% less, at least 90% less, at least 95% less, at least 100% less, at least 150% less, or at least 200% less, at least 250% less, or at least 300%, at least 400% less, or at least 500% less than when a reference pharmaceutical composition is used, for example, to administer the AAV comprising the expression cassette encoding the transgene by suprachoroidal administration, by subretinal administration, or by intravitreous administration.

In some embodiments, a suprachoroidal administration of a pharmaceutical composition (e.g., a composition comprising an AAV comprising an expression cassette encoding a transgene) that is viscous (e.g., relatively viscous, medium to super high viscosity, or more viscous than water, or more viscous than a control solution, or more viscous than a solution commonly used for subretinal administration) or elastic and/or gelled at about 32-35° C. results in a circumferential spread that is at least 2 times less, at least 3 times less, at least 4 times less, at least 5 times less, at least 6 times less, at least 7 times less, at least 8 times less, at least 9 times less, at least 10 times less, at least 15 times less, at least 20 times less, at least 50 times less, at least 100 times less, at least 5% less, at least 10% less, at least 15% less, at least 20% less, at least 25% less, at least 30% less, at least 35% less, at least 40%, at least 45% less, at least 50% less, at least 55% less, at least 60% less, at least 65% less, at least 70% less, at least 75% less, at least 80% less, at least 85% less, at least 90% less, at least 95% less, at least 100% less, at least 150% less, or at least 200% less, at least 250% less, or at least 300%, at least 400% less, or at least 500% less than when the same pharmaceutical composition is used, for example, to administer the AAV comprising the expression cassette encoding the transgene by subretinal administration or by intravitreous administration.

In some embodiments, the circumferential spread can be determined 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after the pharmaceutical composition or the reference pharmaceutical composition is administered.

### 4.2.3 SCS Thickness

In some embodiments, localization can be determined by evaluating SCS thickness after a pharmaceutical composition is administered to a subject. In some embodiments, a pharmaceutical composition increases the thickness of the SCS after the pharmaceutical composition is injected in the SCS. In some embodiments, an SCS expands to accommodate the infusion of a pharmaceutical composition that has low viscosity and/or elastic modulus (G′) and/or is not gelled at about 32-35° C. In some embodiments, the infusion of a greater volume of the low-viscosity and/or low-elastic modulus and/or non-gelled pharmaceutical composition does not cause further expansion of the SCS. In some embodiments, the greater volume of the low-viscosity and/or low elastic modulus fluid formulation is accommodated by increasing the area of fluid spread in the SCS without further expanding the SCS. In some embodiments, the infusion into the SCS of a pharmaceutical composition that is viscous and/or elastic and/or gelled at about 32-35° C. can expand SCS thickness beyond the SCS thickness achieved when a low-viscosity and/or low elastic modulus and/or non-gelled pharmaceutical composition is infused into the SCS. In some embodiments, increasing the SCS thickness with a pharmaceutical composition which is viscous and/or gelled at about 32-35° C. may ease access to the SCS, thereby easing or permitting the disposal of a device in the SCS. In some embodiments, expanding the SCS thickness allows for the pharmaceutical composition and/or the AAV encoded transgene to remain at the site of injection (localized) for a longer period of time. In some embodiments, a pharmaceutical composition that is viscous and/or elastic and/or gelled at about 32-35° C. increases the thickness at or near the site of injection for a longer period of time as compared to a pharmaceutical composition that is non-viscous or has low viscosity and/or low elastic modulus and/or is not gelled at about 32-35° C. In some embodiments, a pharmaceutical composition that is more viscous and/or more elastic and/or gelled at about 32-35° C. increases the thickness at or near the site of injection for a longer period of time as compared to a pharmaceutical composition that is less viscous and/or less elastic and/or not gelled at about 32-35° C. In some embodiments, the thickness at the site of injection after the pharmaceutical composition is administered to the SCS is equal to or higher than the thickness at the site of injection of a reference pharmaceutical composition after the reference pharmaceutical composition is administered subretinally or intravitreously. In some embodiments, the thickness at the site of injection of the pharmaceutical composition after the pharmaceutical composition is administered to the SCS is equal to or higher than the thickness at the site of injection of a reference pharmaceutical composition after the reference pharmaceutical composition is administered to the SCS.

In some embodiments, a suprachoroidal administration of a pharmaceutical composition that is viscous (e.g., relatively viscous, medium to super high viscosity, or more viscous than water, or more viscous than a control solution, or more viscous than a solution commonly used for subretinal administration) and/or elastic and/or gelled at about 32-35° C. (e.g., a composition comprising an AAV comprising an expression cassette encoding a transgene) results in an increase in the SCS thickness that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a pharmaceutical composition that less viscous and/or less elastic and/or not gelled at about 32-35° C. is used, for example, to administer the AAV comprising the expression cassette encoding the transgene by suprachoroidal administration.

In some embodiments, a suprachoroidal administration of a pharmaceutical composition (e.g., a composition comprising an AAV comprising an expression cassette encoding a transgene) that is more viscous and/or more elastic and/or gelled at about 32-35° C. results in an increase in thickness at or near the site of injection that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a pharmaceutical composition which is less viscous and/or less elastic and/or not gelled at about 32-35° C. is used, for example, to administer the AAV comprising the expression cassette encoding the transgene by subretinal administration or by intravitreous administration.

In some embodiments, a suprachoroidal administration of a pharmaceutical composition (e.g., a composition comprising an AAV comprising an expression cassette encoding a transgene) that is viscous (e.g., relatively viscous, medium to super high viscosity, or more viscous than water, or more viscous than a control solution, or more viscous than a solution commonly used for subretinal administration) and/or elastic and/or gelled at about 32-35° C. results in an increase in thickness at or near the site of injection that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when the same pharmaceutical composition is used, for example, to administer the AAV comprising the expression cassette encoding the transgene by subretinal administration or by intravitreous administration.

In some embodiments, the thickness obtained at the site of injection after a pharmaceutical composition (e.g., a pharmaceutical composition comprising an AAV comprising an expression cassette encoding a transgene) that is more viscous and/or more elastic and/or gelled at about 32-35° C. is administered by suprachoroidal injection is greater than after a pharmaceutical composition that is comparably less viscous and/or less elastic and/or not gelled at about 32-35° C. is administered by suprachoroidal injection. In some embodiments, the thickness obtained at the site of injection after a pharmaceutical composition (e.g., a pharmaceutical composition comprising an AAV comprising an expression cassette encoding a transgene) that is more viscous and/or more elastic and/or gelled at about 32-35° C. is administered by suprachoroidal injection is greater than after a pharmaceutical composition that is comparably less viscous and/or less elastic and/or not gelled at about 32-35° C. administered by subretinal injection or by intravitreous injection. In some embodiments, the thickness obtained at the site of injection after a pharmaceutical composition (e.g., a pharmaceutical composition comprising an AAV comprising an expression cassette encoding a transgene) that is viscous and/or gelled at about 32-35° C. is administered by suprachoroidal injection is greater than after the same pharmaceutical composition administered by subretinal administration or by intravitreous administration.

In some embodiments, the thickness at or near the site of injection (e.g., thickness at or near the SCS) can be determined 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after the pharmaceutical composition or the reference pharmaceutical composition is administered.

### 4.2.4 Vasodilation and Vascular Leakage

In some embodiments, a level of VEGF-induced vasodilation and/or vascular leakage after the pharmaceutical composition is administered to the SCS is equal to or less than a level of VEGF-induced vasodilation and/or vascular leakage after a reference pharmaceutical composition is administered subretinally or intravitreously. In some embodiments, a level of VEGF-induced vasodilation and/or vascular leakage after the pharmaceutical composition is administered to the SCS is equal to or lower than a level of VEGF-induced vasodilation and/or vascular leakage after the reference pharmaceutical composition is administered to the SCS. In some embodiments, a pharmaceutical composition (e.g., a composition comprising an AAV comprising an expression cassette encoding a transgene) results in a decreased level of VEGF-induced vasodilation and/or vascular leakage after the same pharmaceutical composition is administered to the SCS as compared to after the pharmaceutical composition is administered via a subretinal administration or via an intravitreous administration. In some embodiments, a pharmaceutical composition results in a decreased level of VEGF-induced vasodilation and/or vascular leakage after the pharmaceutical composition is administered to the SCS as compared to after a comparable (less viscous at about 32-35° C.) pharmaceutical composition is administered via a subretinal administration, via an intravitreous administration, or to the SCS. In some embodiments, the VEGF-induced vasodilation and/or vascular leakage is decreased by at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%. In some embodiments, the transgene is an anti-human vascular endothelial growth factor (anti-VEGF) antibody.

In some embodiments, the VEGF-induced vasodilation and/or vascular leakage is determined about 30 minutes, 1 hour, 2 hours, 4 hours, 6 hours, 8 hours, 10 hours, 12 hours, 14 hours, 15 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or at most about 400 days after administration.

### 4.2.5 Rate of Transduction (or Rate of Infection) at Site of Injection

In some embodiments, the rate of infection at the site of transduction (or rate of injection) after a pharmaceutical composition is administered in the SCS is equal to or higher as compared to the rate of transductions (or rate of infection) at a site of injection after the same pharmaceutical composition is administered via a subretinal administration or via an intravenous administration. In some embodiments, the rate of transduction (or rate of infection) at the site of injection after a pharmaceutical composition is administered in the SCS is equal to or higher as compared to the rate of transduction (or rate of infection) at the site of injection after a comparable (e.g., less viscous and/or not gelled at about 32-35° C.) pharmaceutical composition is administered via a subretinal, or intravenous administration, or to the SCS. In some embodiments, the pharmaceutical composition has a higher viscosity and/or elastic modulus (G′) and/or is gelled at about 32-35° C. than the reference pharmaceutical composition (a pharmaceutical composition that is comparably less viscous and/or less elastic and/or not gelled at about 32-35° C.). In some embodiments, the pharmaceutical composition and the reference pharmaceutical composition have the same vector genome concentration. In some embodiments, the pharmaceutical composition and the reference pharmaceutical composition have the same amount of genome copies. In some embodiments, the increase in the rate of transduction (or rate of infection) at the site of injection is an increase of at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%

In some embodiments, a level of AAV at the site of injection is equal to or higher after the pharmaceutical composition is administered suprachoroidally as compared to a level of AAV at the site of injection after the same pharmaceutical composition is administered via a subretinal administration or via an intravenous administration. In some embodiments, a level of AAV at the site of injection after the pharmaceutical composition is administered suprachoroidally is equal to or higher as compared to a level of AAV at the site of injection after a comparable (e.g., less viscous and/or less elastic and/or not gelled at about 32-35° C.) pharmaceutical composition is administered via a subretinal, or intravenous administration, or to the SCS. In some embodiments, the pharmaceutical composition has a higher viscosity and/or elastic modulus (G′) and/or is gelled at about 32-35° C. than the reference pharmaceutical composition. In some embodiments, the pharmaceutical composition and the reference pharmaceutical composition (a pharmaceutical composition that is comparably less viscous and/or less elastic and/or not gelled at about 32-35° C.) have the same vector genome concentration. In some embodiments, the pharmaceutical composition and a reference pharmaceutical composition have the same amount of genome copies. In some embodiments, the increase in the level of AAV at the site of injection is an increase of at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

In some embodiments, the AAV level or the rate of transduction (or rate of infection) is determined about 30 minutes, 1 hour, 2 hours, 4 hours, 6 hours, 8 hours, 10 hours, 12 hours, 14 hours, 15 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or at most about 400 days after administration.

### 4.2.6 Transgene Expression

In some embodiments, the concentration of a transgene product is at least equal to or higher after a pharmaceutical composition is injected in the SCS as compared to after a reference (e.g., less viscous and/or less elastic and/or not gelled at about 32-35° C.) pharmaceutical composition is injected in the SCS. In some embodiments, the concentration of a transgene product is at least equal to or higher after a pharmaceutical composition is injected in the SCS as compared to after a reference (less viscous and/or less elastic and/or not gelled at about 32-35° C.) pharmaceutical composition is injected by subretinal injection or by intravitreous injection. In some embodiments, the concentration of a transgene product is at least equal to or higher after a pharmaceutical composition is injected in the SCS as compared to after the same pharmaceutical composition is injected by subretinal injection or by intravitreous injection.

In some embodiments, a transgene product (e.g., concentration of the transgene product) is detected in an eye (e.g., vitreous humor) for a longer period of time after a pharmaceutical composition is injected in the SCS as compared to after a comparable (less viscous and/or less elastic and/or not gelled at about 32-35° C.) pharmaceutical composition is injected in the SCS. In some embodiments, a transgene product (e.g., concentration of the transgene product) is detected in an eye (e.g., vitreous humor) for a longer period of time after a pharmaceutical composition is injected in the SCS as compared to after a reference (less viscous and/or less elastic and/or not gelled at about 32-35° C.) pharmaceutical composition is injected by subretinal injection or by intravitreous administration. In some embodiments, a transgene product (e.g., concentration of the transgene product) is detected in an eye (e.g., vitreous humor) for a longer period of time after a pharmaceutical composition is injected in the SCS as compared to after the same (or similar viscosity and/or elastic modulus (G′) at about 32-35° C.) pharmaceutical composition is injected by subretinal injection or by intravitreous injection.

In some embodiments, the longer period of time is at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days longer. In some embodiments, the longer period of time is about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days longer.

In some embodiments, the transgene is detected in an eye (e.g., vitreous humor) for period of time, after the pharmaceutical composition is administered in the SCS, that is at least about or about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after the administration.

In some embodiments, the transgene is detected in an eye (e.g., vitreous humor) for a period of time (e.g., after the reference pharmaceutical composition is administered via subretinal administration or via intravitreous administration or to the SCS; or after the pharmaceutical composition is administered via subretinal or via intravitreous administration) that is at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, or 100 days after administration.

In some embodiments, the concentration of a transgene product in an eye (e.g., vitreous humor) can be determined about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after the pharmaceutical composition or the reference pharmaceutical composition is administered.

In some embodiments, a suprachoroidal administration of a pharmaceutical composition (e.g., a composition comprising an AAV comprising an expression cassette encoding a transgene) that is viscous (e.g., relatively viscous, medium to super high viscosity, or more viscous than water, or more viscous than a control solution, or more viscous than a solution commonly used for subretinal administration) and/or more elastic and/or gelled at about 32-35° C. results in a higher concentration of the transgene that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than after a pharmaceutical composition that is comparably less viscous and/or less elastic and/or not gelled at about 32-35° C. is used, for example, to administer the AAV comprising the expression cassette encoding the transgene by suprachoroidal administration.

In some embodiments, a suprachoroidal administration of a pharmaceutical composition (e.g., a composition comprising an AAV comprising an expression cassette encoding a transgene) that is more viscous and/or more elastic and/or gelled at about 32-35° C. results in a higher concentration of the transgene that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a pharmaceutical composition (a reference pharmaceutical composition) that is comparably less viscous and/or less elastic and/or not gelled at about 32-35° C. is used, for example, to administer the AAV comprising the expression cassette encoding the transgene by subretinal administration or by intravitreous administration.

In some embodiments, a suprachoroidal administration of a pharmaceutical composition (e.g., a composition comprising an AAV comprising an expression cassette encoding a transgene) that is viscous (e.g., relatively viscous, medium to super high viscosity, or more viscous than water, or more viscous than a control solution, or more viscous than a solution commonly used for subretinal administration) and/or elastic and/or gelled at about 32-35° C. results in a higher concentration of the transgene that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when the same pharmaceutical composition is administered via subretinal administration or via intravitreous administration.

In some embodiments, the concentration of the transgene after a pharmaceutical composition (e.g., a pharmaceutical composition comprising an AAV comprising an expression cassette encoding a transgene) that is more viscous and/or more elastic and/or gelled at about 32-35° C. is administered by suprachoroidal injection is greater than after a pharmaceutical composition that is comparably less viscous and/or less elastic and/or not gelled at about 32-35° C. is administered by suprachoroidal injection. In some embodiments, the concentration of the transgene after a pharmaceutical composition (e.g., a pharmaceutical composition comprising an AAV comprising an expression cassette encoding a transgene) that is more viscous and/or more elastic and/or gelled at about 32-35° C. is administered by suprachoroidal injection is greater than after a pharmaceutical composition that is comparably less viscous and/or less elastic and/or not gelled at about 32-35° C. is administered by subretinal administration or via intravitreous administration. In some embodiments, the concentration of the transgene after a pharmaceutical composition (e.g., a pharmaceutical composition comprising an AAV comprising an expression cassette encoding a transgene) that is viscous at about 32-35° C. is administered by suprachoroidal injection is greater than after the same pharmaceutical composition is administered by subretinal administration or via intravitreous administration.

### 4.2.7 Other Functional Properties

In some embodiments, the pharmaceutical composition described herein has a desired viscosity and/or elastic modulus (G′) that is suitable for suprachoroidal injection. In some embodiments, the recombinant AAV in the pharmaceutical composition is at least as stable as the recombinant AAV in a reference pharmaceutical composition (or a comparable pharmaceutical composition). In some embodiments, the recombinant AAV in the pharmaceutical composition is at least 50% as stable as the recombinant AAV in a reference pharmaceutical composition (or a comparable pharmaceutical composition). In some embodiments, the recombinant AAV in the pharmaceutical composition has at least the same or a comparable aggregation level as the recombinant AAV in a reference pharmaceutical composition. In some embodiments, the recombinant AAV in the pharmaceutical composition has at least the same or a comparable infectivity level as the recombinant AAV in a reference pharmaceutical composition. In some embodiments, the recombinant AAV in the pharmaceutical composition has at least the same or a comparable free DNA level as the recombinant AAV in a reference pharmaceutical composition. In some embodiments, the recombinant AAV in the pharmaceutical composition has at least the same or a comparable in vitro relative potency (IVRP) as the recombinant AAV in a reference pharmaceutical composition. In some embodiments, the recombinant AAV in the pharmaceutical composition has at least the same or a comparable change in size level as the recombinant AAV in a reference pharmaceutical composition.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times more stable to freeze/thaw cycles than the same recombinant AAV in a reference pharmaceutical composition. In some embodiments, the recombinant AAV in the pharmaceutical composition is at least about 50%, 55%, 60%, 65%, 70%, 75%, 80%, 85%, 90%, 95%, or 100% as stable to freeze/thaw cycles as the same recombinant AAV in a reference pharmaceutical composition. In certain embodiments, the stability of the recombinant AAV is determined by an assay or assays disclosed in Section 4.6 and Section 5.

In certain embodiments, the recombinant AAV in the pharmaceutical composition exhibits at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times more infectivity than the same recombinant AAV in a reference pharmaceutical composition. In some embodiments, the recombinant AAV in the pharmaceutical composition has at least about 50%, 55%, 60%, 65%, 70%, 75%, 80%, 85%, 90%, 95%, or 100% the infectivity of the same recombinant AAV in a reference pharmaceutical composition. In certain embodiments, the virus infectivity of the recombinant AAV is determined by an assay or assays disclosed in the present disclosure. In certain embodiments, the size of the recombinant AAV is determined by an assay or assays disclosed in Section 4.6 and Section 5. In certain embodiments, the size is measured prior to or after freeze/thaw cycles.

In certain embodiments, the recombinant AAV in the pharmaceutical composition exhibits at least 2%, 5%, 7%, 10%, 12%, 15%1, 17%, 20%, 25%, 30%, 35%, 40%, 45%, 50%, 100%, 2 times, 3 times, 5 times, 10 times, 100 times, or 1000 times less aggregation than the same recombinant AAV in a reference pharmaceutical composition. In certain embodiments, the aggregation of the recombinant AAV is determined by an assay or assays disclosed in the present disclosure. In certain embodiments, the aggregation is measured prior to or after freeze/thaw cycles. In certain embodiments, the aggregation of the recombinant AAV is determined by an assay or assays disclosed in Section 4.6.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times more stable over a period of time (e.g., when stored at −20° C. or at 37° C.), for example, at least about or about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, 12 months, about 15 months, about 18 months, about 24 months, about 2 years, about 3 years, about 4 years than the same recombinant AAV in a reference pharmaceutical composition. In some embodiments, the recombinant AAV in the pharmaceutical composition is at least about 50%, 55%, 60%, 65%, 70%, 75%, 80%, 85%, 90%, 95%, or 100% as stable over a period of time as the same recombinant AAV in a reference pharmaceutical composition. In certain embodiments, the stability over a period of time of the recombinant AAV is determined by an assay or assays disclosed in the present disclosure. In certain embodiments, the stability over a period of time of the recombinant AAV is determined by an assay or assays disclosed in Section 4.6 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in in vitro relative potency (IVRP) than the same recombinant AAV in a reference pharmaceutical composition (e.g., when stored at −20° C. or at 37° C.). In some embodiments, the recombinant AAV in the pharmaceutical composition has about the same in vitro relative potency (IVRP) as the same recombinant AAV in a reference pharmaceutical composition. In some embodiments, the recombinant AAV in the pharmaceutical composition has about 50%, 55%, 60%, 65%, 70%, 75%, 80%, 85%, 90%, 95%, or 100% in vitro relative potency (IVRP) as the same recombinant AAV in a reference pharmaceutical composition. In certain embodiments, the in vitro relative potency (IVRP) of the recombinant AAV is determined by an assay or assays disclosed in the present disclosure. In certain embodiments, the in vitro relative potency (IVRP) is measured prior to or after freeze/thaw cycles. In certain embodiments, the in vitro relative potency (IVRP) of the recombinant AAV is determined by an assay or assays disclosed in Section 4.6.

In certain embodiments, the recombinant AAV in the pharmaceutical composition has at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times less free DNA than the same recombinant AAV in a reference pharmaceutical composition. In some embodiments, the recombinant AAV in the pharmaceutical composition has about the same amount of free DNA as the same recombinant AAV in a reference pharmaceutical composition. In some embodiments, the recombinant AAV in the pharmaceutical composition has about not more than two times the amount of free DNA as the same recombinant AAV in a reference pharmaceutical composition. In some embodiments, the recombinant AAV in the pharmaceutical composition has about 50%, 55%, 60%, 65%, 70%, 75%, 80%, 85%, 90%, 95%, or 100% the amount of free DNA as the same recombinant AAV in a reference pharmaceutical composition. In some embodiments, the recombinant AAV in the pharmaceutical composition has at least about 50% more, about 25% more, about 15% more, about 10% more, about 5% more, about 4% more, about 3% more, about 2% more, about 1% more, about 0% more, about 1% less, about 2% less, about 5% less, about 7% less, about 10% less, about 2 times more, about 3 times more, about 2 times less, or about 3 times less free DNA than the same recombinant AAV in a reference pharmaceutical composition. In certain embodiments, the free DNA of the recombinant AAV is determined by an assay or assays disclosed in Section 4.6 and Section 5.

In certain embodiments, the recombinant AAV in the pharmaceutical composition has at most 20%, 15%, 10%, 8%, 5%, 4%, 3%, 2%, or 1% change in size over a period of time (e.g., when stored at −20° C. or at 37° C.), for example, at least about or about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in the present disclosure. In certain embodiments, the size is measured prior to or after freeze/thaw cycles. In certain embodiments, the size of the recombinant AAV is determined by an assay or assays disclosed in Section 4.6.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times more stable than the same recombinant AAV in a reference pharmaceutical composition (e.g., when stored at −20° C. or at 37° C.). In some embodiments, the recombinant AAV in the pharmaceutical composition is about as stable as the same recombinant AAV in a reference pharmaceutical composition (e.g., when stored at −20° C. or at 37° C.). In some embodiments, the recombinant AAV in the pharmaceutical composition is at least about 50%, 55%, 60%, 65%, 70%, 75%, 80%, 85%, 90%, 95%, or 99% as stable as the same recombinant AAV in a reference pharmaceutical composition (e.g., when stored at −20° C. or at 37° C.). In certain embodiments, the stability of the recombinant AAV is determined by an assay or assays disclosed in Section 4.6.

In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months without loss of stability as determined, e.g. by an assay or assays disclosed in Section 4.6. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months at 4° C. without loss of stability. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months at ≤60° C. without loss of stability. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months at −80° C. without loss of stability. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months at 4° C. after having been stored at −20° C. for 1, 2, 3, 4, 5, 6, 7, 8, 9, 10, or 12 months without loss of stability.

In certain embodiments, a pharmaceutical composition provided herein is capable of being first stored for 1, 2, 3, 4, 5, 6, 7, 8, 9, 10, 12, 13, 14, 15, 16, 17, 18, 19, 20, 21, 22, 23, or 24 months at −80° C., then being thawed and, after thawing, being stored at 2-10° C., 4-8° C., 2° C., 3° C., 4° C., 5° C., 6° C., 7° C., 8° C. or 9° C. for 1, 2, 3, 4, 5, 6, 7, 8, 9, 10, or 12 additional months without loss of stability as determined, e.g., by an assay or assays disclosed in Section 4.6. In certain embodiments, a pharmaceutical composition provided herein is capable of being first stored for 1, 2, 3, 4, 5, 6, 7, 8, 9, 10, 12, 13, 14, 15, 16, 17, 18, 19, 20, 21, 22, 23, or 24 months at −80° C., then being thawed and, after thawing, being stored at about 4° C. for 1, 2, 3, 4, 5, 6, 7, 8, 9, 10, or 12 additional months without loss of stability as determined, e.g., by an assay or assays disclosed in Section 4.6. In certain embodiments, a pharmaceutical composition provided herein is capable of being first stored for 1, 2, 3, 4, 5, 6, 7, 8, 9, 10, 12, 13, 14, 15, 16, 17, 18, 19, 20, 21, 22, 23, or 24 months at ≤60° C., then being thawed and, after thawing, being stored at about 4° C. for 1, 2, 3, 4, 5, 6, 7, 8, 9, 10, or 12 additional months without loss of stability as determined, e.g., by an assay or assays disclosed in Section 4.6.

Effects of the methods or pharmaceutical compositions provided herein may be monitored by measuring signs of vision loss, infection, inflammation and other safety events, including retinal detachment. In some embodiments, different pharmaceutical compositions having different viscosity and/or elastic modulus (G′) (e.g., ranging from low viscosity to very high viscosity) at about 32-35° C. can be used to deliver the vector in the SCS. In some embodiments, vectors delivered using a pharmaceutical composition that has medium to high viscosity and/or elastic modulus (G′) at about 32-35° C. are more effective than vectors delivered using a pharmaceutical composition that has a low viscosity and/or elastic modulus (G′) at about 32-35° C. (e.g., when administered in the SCS). In some embodiments, vectors delivered using a formulation that has medium to high viscosity and/or elastic modulus (G′) at about 32-35° C. results in improved vision as compared to vectors delivered using a formulation which has low viscosity and/or elastic modulus (G′) at about 32-35° C.

Effects of the methods or pharmaceutical compositions provided herein may also be measured by a change from baseline in National Eye Institute Visual Functioning Questionnaire, the Rasch-scored version (NEI-VFQ-28-R) (composite score; activity limitation domain score; and socio-emotional functioning domain score). In some embodiments, effects of the methods provided herein may also be measured by a change from baseline in National Eye Institute Visual Functioning Questionnaire 25-item version (NEI-VFQ-25) (composite score and mental health subscale score). In some embodiments, effects of the methods provided herein may also be measured by a change from baseline in Macular Disease Treatment Satisfaction Questionnaire (MacTSQ) (composite score; safety, efficacy, and discomfort domain score; and information provision and convenience domain score).

In specific embodiments, the efficacy of a method or vector (vector formulation) described herein is reflected by an improvement in vision at about 4 weeks, 12 weeks, 6 months, 12 months, 24 months, 36 months, or at other desired timepoints. In a specific embodiment, the improvement in vision is characterized by an increase in BCVA, for example, an increase by 1 letter, 2 letters, 3 letters, 4 letters, 5 letters, 6 letters, 7 letters, 8 letters, 9 letters, 10 letters, 11 letters, or 12 letters, or more. In a specific embodiment, the improvement in vision is characterized by a 5%, 10%, 15%, 20%, 30%, 40%, 50% or more increase in visual acuity from baseline.

In specific embodiments, there is no inflammation in the eye after treatment or little inflammation in the eye after treatment (for example, an increase in the level of inflammation by 10%, 5%, 2%, 1% or less from baseline).

### 4.3 Dosage and Mode of Administration

In one aspect, provided herein is a method of suprachoroidal administration for treating a pathology of the eye, comprising administering to the suprachoroidal space in the eye of a human subject in need of treatment a recombinant viral vector comprising a nucleotide sequence encoding a therapeutic product such that the therapeutic product is expressed and results in treatment of the pathology of the eye. In certain embodiments, the administering step is by injecting the recombinant viral vector into the suprachoroidal space using a suprachoroidal drug delivery device. In certain embodiments, the suprachoroidal drug delivery device is a microinjector. In some embodiments, a pharmaceutical composition or a reference pharmaceutical composition provided herein is suitable for administration by one, two or more routes of administration (e.g., suitable for suprachoroidal and subretinal administration).

In certain embodiments, the vector genome concentration (VGC) of the pharmaceutical composition (or the reference pharmaceutical composition) is about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 3.2×10¹¹ GC/mL, about 6.2×10¹¹ GC/mL, about 6.5×10¹¹ GC/mL, about 1×10¹² GC/mL, about 2.5×10¹² GC/mL, about 3×10¹² GC/mL, about 5×10¹² GC/mL, about 1.5×10¹³ GC/mL, about 2×10¹³ GC/mL or about 3×10¹³ GC/mL.

In certain embodiments, the vector genome concentration (VGC) of the pharmaceutical composition (or the reference pharmaceutical composition) is about 3×10⁹ GC/mL, 4×10⁹ GC/mL, 5×10⁹ GC/mL, 6×10⁹ GC/mL, 7×10⁹ GC/mL, 8×10⁹ GC/mL, 9×10⁹ GC/mL, about 1×10¹⁰ GC/mL, about 2×10¹⁰ GC/mL, about 3×10¹⁰ GC/mL, about 4×10¹⁰ GC/mL, about 5×10¹⁰ GC/mL, about 6×10¹⁰ GC/mL, about 7×10¹⁰ GC/mL, about 8×10¹⁰ GC/mL, about 9×10¹⁰ GC/mL, about 1×10¹¹ GC/mL, about 2×10¹¹ GC/mL, about 3×10¹¹ GC/mL, about 4×10¹¹ GC/mL, about 5×10¹¹ GC/mL, about 6×10¹¹ GC/mL, about 7×10¹¹ GC/mL, about 8×10¹¹ GC/mL, about 9×10¹¹ GC/mL, about 1×10¹² GC/mL, about 2×10¹² GC/mL, about 3×10¹² GC/mL, about 4×10¹² GC/mL, about 5×10¹² GC/mL, about 6×10¹² GC/mL, about 7×10¹² GC/mL, about 8×10¹² GC/mL, about 9×10¹² GC/mL, about 1×10¹³ GC/mL, about 1.5×10¹³ GC/mL, about 2×10¹³ GC/mL, about 3×10¹³ GC/mL.

In some embodiments, the volume of the pharmaceutical composition is any volume capable of reducing the minimum force to separate the sclera and choroid. In some embodiments, the volume of the pharmaceutical composition is about 50 μL to about 1000 μL, 50 μL to about 500 μL, 50 μL to about 400 μL, 50 μL to about 350 μL, 50 μL to about 300 μL, about 50 μL to about 275 μL, about 50 μL to about 250 μL, about 50 μL to about 225 μL, about 50 μL to about 200 μL, about 50 μL to about 175 μL, about 50 μL to about 150 μL, about 60 μL to about 140 μL, about 70 μL to about 130 μL, about 80 μL to about 120 μL, about 90 μL to about 110 μL, or about 100 μL.

Currently available technologies for suprachoroidal space (SCS) delivery exist. Preclinically, SC injections have been achieved with scleral flap technique, catheters and standard hypodermic needles, as well as with microneedles. A hollow-bore 750 um-long microneedle (Clearside Biomedical, Inc.) can be inserted at the pars, and has shown promise in clinical trials. A microneedle designed with force-sensing technology can be utilized for SC injections, as described by Chitnis, et al. (Chitnis, G. D., et al. A resistance-sensing mechanical injector for the precise delivery of liquids to target tissue. Nat Biomed Eng 3, 621-631 (2019). https://doi.org/10.1038/s41551-019-0350-2). Oxular Limited is developing a delivery system (Oxulumis) that advances an illuminated cannula in the suprachoroidal space. The Orbit device (Gyroscope) is a specially-designed system enabling cannulation of the suprachoroidal space with a flexible cannula. A microneedle inside the cannula is advanced into the subretinal space to enable targeted dose delivery. Ab interno access to the SCS can also be achieved using micro-stents, which serve as minimally-invasive glaucoma surgery (MIGS) devices. Examples include the CyPass® Micro-Stent (Alcon, Fort Worth, Texas, US) and iStent® (Glaukos), which are surgically implanted to provide a conduit from the anterior chamber to the SCS to drain the aqueous humor without forming a filtering bleb. Other devices contemplated for suprachoroidal delivery include those described in UK Patent Publication No. GB 2531910A and U.S. Pat. No. 10,912,883 B2.

In some embodiments, the suprachoroidal drug delivery device is a syringe with a 1 millimeter 30 gauge needle. In some embodiments, the syringe has a larger circumference (e.g., 29 gauge needle). During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of transgene protein from all retinal cell layers and choroidal cells. Using this type of device and procedure allows for a quick and easy in-office procedure with low risk of complications.

In some embodiments, a microneedle or syringe is selected based on the viscosity of a pharmaceutical composition. In some embodiments, a microneedle is selected based on the pressure resulted in the eye (e.g., in the SCS) when a pharmaceutical composition is administered. For example, a pharmaceutical composition having medium or high viscosity and/or elastic modulus (G′) at about 32-35° C. may benefit from the use of a wider microneedle for injection. In some embodiments, the pressure in the SCS is lower when a wider microneedle is used as compared to the pressure obtained when a narrower microneedle is used. In some embodiments, 10 gauge needle, 11 gauge needle, 12 gauge needle, 13 gauge needle, 14 gauge needle, 15 gauge needle, 16 gauge needle, 17 gauge needle, 18 gauge needle, 19 gauge needle, 20 gauge needle, 21 gauge needle, 22 gauge needle, 23 gauge needle, 24 gauge needle, 25 gauge needle, 26 gauge needle, 27 gauge needle, 28 gauge needle, 29 gauge needle, 30 gauge needle, 31 gauge needle, 32 gauge needle, 33 gauge needle, or 34 gauge needle is used. In some embodiments, a 27 gauge needle is used. In some embodiments, a 28 gauge needle is used. In some embodiments, a 29 gauge needle is used. In some embodiments, a 30 gauge needle is used. In some embodiments, a 31 gauge needle is used. In some embodiments, a gauge that is smaller than a 27 gauge needle is used. In some embodiments, a gauge that is larger than a 27 gauge needle is used. In some embodiments, a gauge that is smaller than a 30 gauge needle is used. In some embodiments, a gauge that is higher than a 30 gauge needle is used.

In some embodiments, the pressure during administration of a pharmaceutical composition is about 10 PSI, 15 PSI, 20 PSI, 25 PSI, 30 PSI, 35 PSI, 40 PSI, 45 PSI, 50 PSI, 55 PSI, 60 PSI, 65 PSI, 70 PSI, 75 PSI, 80 PSI, 85 PSI, 90 PSI, 95 PSI, 100 PSI, 150 PSI, or 200 PSI. In some embodiments, the pressure during administration of a pharmaceutical composition is not greater than about 10 PSI, 15 PSI, 20 PSI, 25 PSI, 30 PSI, 35 PSI, 40 PSI, 45 PSI, 50 PSI, 55 PSI, 60 PSI, 65 PSI, 70 PSI, 75 PSI, 80 PSI, 85 PSI, 90 PSI, 95 PSI, 100 PSI, 150 PSI, or 200 PSI. In some embodiments, the pressure to open the SCS during administration of a pharmaceutical composition is not greater than about 10 PSI, 15 PSI, 20 PSI, 25 PSI, 30 PSI, 35 PSI, 40 PSI, 45 PSI, 50 PSI, 55 PSI, 60 PSI, 65 PSI, 70 PSI, 75 PSI, 80 PSI, 85 PSI, 90 PSI, 95 PSI, 100 PSI, 150 PSI, or 200 PSI. In some embodiments, the pressure during administration of a pharmaceutical composition (or the pressure required to open the SCS) is between 20 PSI and 50 PSI, 20 PSI and 75 PSI, 20 PSI and 40 PSI, 10 PSI and 40 PSI, 10 PSI and 100 PSI, or 10 PSI and 80 PSI. In some embodiments, the pressure decreases as the rate of injection decreases (e.g., pressure decreases from a 4 seconds rate of injection to a 10 seconds rate of injection). In some embodiments, the pressure decreases as the size of the needle increases.

In some embodiments, a pharmaceutical composition provided herein is administered to the human eye with an injection pressure of less than 43 PSI. In some embodiments, a pharmaceutical composition provided herein is administered to the human eye with an injection pressure of a about 43 PSI. In some embodiments, a pharmaceutical composition provided herein is administered to the human eye with an injection pressure of about 43-65 PSI. In some embodiments, a pharmaceutical composition provided herein is administered to the human eye with an injection pressure of about 65 PSI. In some embodiments, a pharmaceutical composition provided herein is administered to the human eye with an injection pressure of less than 65 PSI. In some embodiments, a pharmaceutical composition provided herein is administered to the human eye with an injection pressure of about 65-100 PSI. In some embodiments, a pharmaceutical composition provided herein is administered to the human eye with an injection pressure of about 100 PSI. In some embodiments, a pharmaceutical composition provided herein is administered to the human eye with an injection pressure of less than 100 PSI.

In some embodiments, a pharmaceutical composition provided herein is administered to the human eye in an injection time of about 5-10 seconds. In some embodiments, a pharmaceutical composition provided herein is administered to the human eye in an injection time of about 10-15 seconds. In some embodiments, a pharmaceutical composition provided herein is administered to the human eye in an injection time of about 15-20 seconds. In some embodiments, a pharmaceutical composition provided herein is administered to the human eye in an injection time of about 20-25 seconds. In some embodiments, a pharmaceutical composition provided herein is administered to the human eye in an injection time of about 25-30 seconds. In some embodiments, a pharmaceutical composition provided herein is administered to the human eye in an injection time of less than 30 seconds. In some embodiments, a pharmaceutical composition provided herein is administered to the human eye in an injection time which is about 10%, 20%, 30%, 40%, 50%, 60%, 70%, 80%, or 90% of the length of the gelation time of the composition at extraocular temperature (about 32-35° C.).

Doses that maintain a concentration of the transgene product at a Cmin of at least 0.330 μg/mL in the eye (e.g., Vitreous humor), or 0.110 μg/mL in the Aqueous humour (the anterior chamber of the eye) for three months are desired; thereafter, Vitreous Cmin concentrations of the transgene product ranging from 1.70 to 6.60 μg/mL, and/or Aqueous Cmin concentrations ranging from 0.567 to 2.20 μg/mL should be maintained. However, because the transgene product is continuously produced (under the control of a constitutive promoter or induced by hypoxic conditions when using an hypoxia-inducible promoter), maintenance of lower concentrations can be effective. Transgene concentrations can be measured directly in patient samples of fluid collected from a bodily fluid, ocular fluid, vitreous humor, or the anterior chamber, or estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

In certain embodiments, dosages are measured by genome copies per ml (GC/mL) or the number of genome copies administered to the eye of the patient (e.g., administered suprachoroidally). In some embodiments, 2.4×10¹¹ GC/mL to 1×10¹³ GC/mL are administered, 2.4×10¹¹ GC/mL to 5×10¹¹ GC/mL are administered, 5×10¹¹ GC/mL to 1×10¹² GC/mL are administered, 1×10¹² GC/mL to 5×10¹² GC/mL are administered, or 5×10¹² GC/mL to 1×10¹³ GC/mL are administered. In some embodiments, 1.5×10¹³ GC/mL to 3×10¹³ GC/mL are administered. In some embodiments, about 2.4×10¹¹ GC/mL, about 5×10¹¹ GC/mL, about 1×10¹² GC/mL, about 2.5×10¹² GC/mL, about 5×10¹² GC/mL, about 1×10¹³ GC/mL or about 1.5×10¹³ GC/mL are administered. In some embodiments, 1×10⁹ to 1×10¹² genome copies are administered. In some embodiments, 3×10⁹ to 2.5×10¹¹ genome copies are administered. In specific embodiments, 1×10⁹ to 2.5×10¹¹ genome copies are administered. In specific embodiments, 1×10⁹ to 1×10¹¹ genome copies are administered. In specific embodiments, 1×10⁹ to 5×10⁹ genome copies are administered. In specific embodiments, 6×10⁹ to 3×10¹⁰ genome copies are administered. In specific embodiments, 4×10¹⁰ to 1×10¹¹ genome copies are administered. In specific embodiments, 2×10¹¹ to 1.5×10¹² genome copies are administered. In a specific embodiment, about 3×10⁹ genome copies are administered (which corresponds to about 1.2×10¹⁰ GC/mL in a volume of 250 μl). In another specific embodiment, about 1×10¹⁰ genome copies are administered (which corresponds to about 4×10¹⁰ GC/mL in a volume of 250 μl). In another specific embodiment, about 6×10¹⁰ genome copies are administered (which corresponds to about 2.4×10¹¹ GC/mL in a volume of 250 μl). In another specific embodiment, about 6.4×10¹⁰ genome copies are administered (which corresponds to about 3.2×10¹¹ GC/mL in a volume of 200 μl). In another specific embodiment, about 1.3×10¹¹ genome copies are administered (which corresponds to about 6.5×10¹¹ GC/mL in a volume of 200 μl). In some embodiments, about 6.4×10¹⁰ genome copies are administered per eye, or per dose, or per route of administration. In some embodiments, about 6.4×10¹⁰ genome copies is the total number of genome copies administered. In some embodiments, about 1.3×10¹¹ genome copies are administered per eye, or per dose, or per route of administration. In some embodiments, about 1.3×10¹¹ genome copies is the total number of genome copies administered. In some embodiments, about 2.5×10¹¹ genome copies are administered per eye, or per dose, or per route of administration. In some embodiments, about 2.5×10¹¹ genome copies is the total number of genome copies administered. In some embodiments, about 5×10¹¹ genome copies are administered per eye, or per dose, or per route of administration. In some embodiments, about 5×10¹¹ genome copies is the total number of genome copies administered. In some embodiments, about 3×10¹² genome copies are administered per eye, or per dose, or per route of administration. In some embodiments, about 3×10¹² genome copies is the total number of genome copies administered. In another specific embodiment, about 1.6×10¹¹ genome copies are administered (which corresponds to about 6.2×10¹¹ GC/mL in a volume of 250 μl). In another specific embodiment, about 1.55×10¹¹ genome copies are administered (which corresponds to about 6.2×10¹¹ GC/mL in a volume of 250 μl). In another specific embodiment, about 1.6×10¹¹ genome copies are administered (which corresponds to about 6.4×10¹¹ GC/mL in a volume of 250 μl). In another specific embodiment, about 2.5×10¹¹ genome copies (which corresponds to about 1.0×10¹² in a volume of 250 μl) are administered. In another specific embodiment, about 2.5×10¹¹ genome copies are administered (which corresponds to about 2.5×10¹² GC/mL in a volume of 100 μl). In another specific embodiment, about 5×10¹¹ genome copies are administered (which corresponds to about 5×10¹² GC/mL in a volume of 200 μl). In another specific embodiment, about 1.5×10¹² genome copies are administered (which corresponds to about 1.5×10¹³ GC/mL in a volume of 100 μl). In another specific embodiment, about 3×10¹¹ genome copies are administered (which corresponds to about 3×10¹² GC/mL in a volume of 100 μl). In another specific embodiment, about 6×10¹¹ genome copies are administered (which corresponds to about 3×10¹² GC/mL in a volume of 200 μl). In another specific embodiment, about 6×10¹¹ genome copies are administered (which corresponds to about 6×10¹² GC/mL in a volume of 100 μl).

In certain embodiments, about 6.0×10¹⁰ genome copies per administration, or per eye are administered. In certain embodiments, about 6.4×10¹⁰ genome copies per administration, or per eye are administered. In certain embodiments, about 1.3×10¹¹ genome copies per administration, or per eye are administered. In certain embodiments, about 1.5×10¹¹ genome copies per administration, or per eye are administered. In certain embodiments, about 1.6×10¹¹ genome copies per administration, or per eye are administered. In certain embodiments, about 2.5×10¹¹ genome copies per administration, or per eye are administered. In certain embodiments, about 3×10¹¹ genome copies per administration, or per eye are administered. In certain embodiments, about 5.0×10¹¹ genome copies per administration, or per eye are administered. In certain embodiments, about 6×10¹¹ genome copies per administration, or per eye are administered. In some embodiments, about 1.5×10¹² genome copies are administered per eye, or per dose, or per route of administration. In some embodiments, about 1.5×10¹² genome copies is the total number of genome copies administered.

In certain embodiments, about 3×10¹² genome copies per administration, or per eye are administered. In certain embodiments, about 1.0×10¹² GC/mL per administration, or per eye are administered. In certain embodiments, about 2.5×10¹² GC/mL per administration, or per eye are administered. In certain embodiments, about 3×10¹² GC/mL per administration, or per eye are administered. In certain embodiments, about 3.0×10¹³ genome copies per administration, or per eye are administered. In certain embodiments, up to 3.0×10¹³ genome copies per administration, or per eye are administered.

In certain embodiments, about 1.5×10¹¹ genome copies per administration, or per eye are administered by suprachoroidal injection. In certain embodiments, about 2.5×10¹¹ genome copies per administration, or per eye are administered by suprachoroidal injection. In certain embodiments, about 3×10¹¹ genome copies per administration, or per eye are administered by suprachoroidal injection. In certain embodiments, about 5.0×10¹¹ genome copies per administration, or per eye are administered by suprachoroidal injection. In certain embodiments, about 6×10¹¹ genome copies per administration, or per eye are administered by suprachoroidal injection. In certain embodiments, about 1.5×10¹² genome copies per administration, or per eye are administered by suprachoroidal injection. In certain embodiments, about 3×10¹² genome copies per administration, or per eye are administered by suprachoroidal injection. In certain embodiments, about 2.5×10¹¹ genome copies per eye are administered by a single suprachoroidal injection. In certain embodiments, about 3×10¹¹ genome copies per administration, or per eye are administered by a single suprachoroidal injection. In certain embodiments, about 3×10¹¹ genome copies per administration, or per eye are administered by a single suprachoroidal injection in a volume of 100 μl. In certain embodiments, about 3×10¹¹ genome copies per administration, or per eye are administered by a single suprachoroidal injection in a volume of 200 μl. In certain embodiments, about 3×10¹¹ genome copies per administration, or per eye are administered by double suprachoroidal injections. In certain embodiments, about 3×10¹¹ genome copies per administration, or per eye are administered by double suprachoroidal injections, wherein each injection is in a volume of 50 μl. In certain embodiments, about 3×10¹¹ genome copies per administration, or per eye are administered by double suprachoroidal injections, wherein each injection is in a volume of 100 μl. In certain embodiments, about 5.0×10¹¹ genome copies per administration, or per eye are administered by double suprachoroidal injections. In certain embodiments, about 6×10¹¹ genome copies per administration, or per eye are administered by a single suprachoroidal injection. In certain embodiments, about 6×10¹¹ genome copies per administration, or per eye are administered by a single suprachoroidal injection in a volume of 100 μl. In certain embodiments, about 6×10¹¹ genome copies per administration, or per eye are administered by a single suprachoroidal injection in a volume of 200 μl. In certain embodiments, about 6×10¹¹ genome copies per administration, or per eye are administered by double suprachoroidal injections. In certain embodiments, about 6×10¹¹ genome copies per administration, or per eye are administered by double suprachoroidal injections, wherein each injection is in a volume of 50 μl. In certain embodiments, about 6×10¹¹ genome copies per administration, or per eye are administered by double suprachoroidal injections, wherein each injection is in a volume of 100 μl. In certain embodiments, about 3.0×10¹³ genome copies per administration, or per eye are administered by suprachoroidal injection. In certain embodiments, up to 3.0×10¹³ genome copies per administration, or per eye are administered by suprachoroidal injection. In certain embodiments, about 2.5×10¹² GC/mL per eye are administered by a single suprachoroidal injection in a volume of 100 μl. In certain embodiments, about 2.5×10¹² GC/mL per eye are administered by double suprachoroidal injections, wherein each injection is in a volume of 100 μl. In certain embodiments, about 1.5×10¹³ GC/mL per eye are administered by a single suprachoroidal injection in a volume of 100 μl.

In certain embodiments, the recombinant viral vector is administered by double suprachoroidal injections. In certain embodiments, the first injection in the right eye is administered in the superior temporal quadrant (i.e., between the 10 o'clock and 11 o'clock positions), and the second injection in the same eye is administered in the inferior nasal quadrant (i.e., between the 4 o'clock and 5 o'clock positions). In certain embodiments, the first injection in the right eye is administered in the inferior nasal quadrant (i.e., between the 4 o'clock and 5 o'clock positions), and the second injection in the same eye is administered in the superior temporal quadrant (i.e., between the 10 o'clock and 11 o'clock positions). In certain embodiments, the first injection in the left eye is administered in the superior temporal quadrant (i.e., between the 1 o'clock and 2 o'clock positions), and the second injection in the same eye is administered in the inferior nasal quadrant (i.e., between the 7 o'clock and 8 o'clock positions). In certain embodiments, the first injection in the left eye is administered in the inferior nasal quadrant (i.e., between the 7 o'clock and 8 o'clock positions), and the second injection in the same eye is administered in the superior temporal quadrant (i.e., between the 1 o'clock and 2 o'clock positions).

In certain embodiments, the recombinant viral vector is administered by a single suprachoroidal injection. In certain embodiments, the single injection in the right eye is administered in the superior temporal quadrant (i.e., between the 10 o'clock and 11 o'clock positions). In certain embodiments, the single injection in the right eye is administered in the inferior nasal quadrant (i.e., between the 4 o'clock and 5 o'clock positions). In certain embodiments, the single injection in the left eye is administered in the superior temporal quadrant (i.e., between the 1 o'clock and 2 o'clock positions). In certain embodiments, the single injection in the left eye is administered in the inferior nasal quadrant (i.e., between the 7 o'clock and 8 o'clock positions).

In some embodiments, the pharmaceutical composition or the reference pharmaceutical composition is administered to a human subject (e.g., suprachoroidally, subretinally, or intravitreously) once, twice, three times, four times, five times, six times, seven times, eight times, nine times, ten times, fifteen times, twenty times, twenty five times, or thirty times. In some embodiments, the pharmaceutical composition or the reference pharmaceutical composition is administered to a human subject once in one day, twice in one day, three times in one day, four times in one day, five times in one day, six times in one day, or seven times in one day. In some embodiments, the same amount of AAV genome copies are administered per administration. For example, the same genome copies are administered suprachoroidally, subretinally, or intravitreously. In some embodiments, the same total amount of AAV genome copies are administered. For example, the same total amount of AAV genome copies are administered suprachoroidally, subretinally, or intravitreously regardless of the number of total administrations (e.g., if subretinal administration is performed once and suprachoroidal administration is performed twice, the genome copies in the one subretinal administration is the same as the genome copies in both suprachoroidal administrations combined).

As used herein and unless otherwise specified, the term “about” means within plus or minus 10% of a given value or range

### 4.4 Constructs and Formulations

In some embodiments, the recombinant vectors provided herein comprise the following elements in the following order: a) a constitutive or a hypoxia-inducible promoter sequence, and b) a sequence encoding the transgene (e.g., therapeutic product). In certain embodiments, the recombinant vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the transgene (e.g., an anti-VEGF antigen-binding fragment moiety), i) a second UTR sequence, j) a fourth linker sequence, k) a poly A sequence, l) a fifth linker sequence, and m) a second ITR sequence.

In certain embodiments, the recombinant vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the transgene (e.g., an anti-VEGF antigen-binding fragment moiety), i) a second UTR sequence, j) a fourth linker sequence, k) a poly A sequence, l) a fifth linker sequence, and m) a second ITR sequence, wherein the transgene comprises the signal peptide of VEGF (SEQ ID NO: 5), and wherein the transgene encodes a light chain and a heavy chain sequence separated by a cleavable F/F2A sequence.

In some embodiments, the AAV (AAV viral vectors) provided herein comprise the following elements in the following order: a) a constitutive or a hypoxia-inducible promoter sequence, and b) a sequence encoding the transgene (e.g., an anti-VEGF antigen-binding fragment moiety). In some embodiments, the transgene is a fully human post-translationally modified (HuP™) antibody against VEGF. In some embodiments, the fully human post-translationally modified antibody against VEGF is a fully human post-translationally modified antigen-binding fragment of a monoclonal antibody (mAb) against VEGF (“HuPTMFabVEGFi”). In some embodiments, the HuPTMFabVEGFi is a fully human glycosylated antigen-binding fragment of an anti-VEGF mAb (“HuGlyFabVEGFi”). In an alternative embodiment, full-length mAbs can be used. In some embodiments, the AAV used for delivering the transgene should have a tropism for human retinal cells or photoreceptor cells. Such AAV can include non-replicating recombinant adeno-associated virus vectors (“rAAV”), particularly those bearing an AAV8 capsid are preferred. In a specific embodiment, the viral vector or other DNA expression construct described herein is Construct I, wherein the Construct I comprises the following components: (1) AAV8 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides. In some embodiments, the viral vector comprises a signal peptide. In some embodiments, the signal peptide is MYRMQLLLLIALSLALVTNS (SEQ ID NO: 55). In some embodiments, the signal peptide is derived from IL-2 signal sequence. In some embodiments, the viral vector comprises a signal peptide from any signal peptide disclosed in Table 1, such as MNFLLSWVHW SLALLLYLHH AKWSQA (VEGF-A signal peptide) (SEQ ID NO: 5); MERAAPSRRV PLPLLLLGGL ALLAAGVDA (Fibulin-1 signal peptide) (SEQ ID NO: 6); MAPLRPLLIL ALLAWVALA (Vitronectin signal peptide) (SEQ ID NO: 7); MRLLAKIICLMLWAICVA (Complement Factor H signal peptide) (SEQ ID NO: 8); MRLLAFLSLL ALVLQETGT (Opticin signal peptide) (SEQ ID NO: 9); MKWVTFISLLFLFSSAYS (Albumin signal peptide) (SEQ ID NO: 22); MAFLWLLSCWALLGTTFG (Chymotrypsinogen signal peptide) (SEQ ID NO: 23); MYRMQLLSCIALILALVTNS (Interleukin-2 signal peptide) (SEQ ID NO: 24); MNLLLILTFVAAAVA (Trypsinogen-2 signal peptide) (SEQ ID NO: 25); or MYRMQLLLLIALSLALVTNS (mutant Interleukin-2 signal peptide) (SEQ ID NO: 55). In another specific embodiment, the viral vector or other DNA expression construct described herein is Construct II, wherein the Construct II comprise the following components: (1) AAV2 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides. In some embodiments, the anti-hVEGF antibody comprises a heavy chain comprising the amino acid sequence of SEQ ID NO:2 or SEQ ID NO:4, and a light chain comprising the amino acid sequence of SEQ ID NO:1 or SEQ ID NO: 3.

In some embodiments, the viral vector or other expression construct suitable for packaging in an AAV capsid, comprises (1) AAV inverted terminal repeats (ITRs) flank the expression cassette; (2) regulatory control elements, consisting essentially of one or more enhancers and/or promoters, d) a poly A signal, and e) optionally an intron; and (3) a transgene providing (e.g., coding for) one or more RNA or protein products of interest.

In some aspects, the disclosure provides for a nucleic acid for use, wherein the nucleic acid encodes a therapeutic product operatively linked to a promoter or enhancer-promoter described herein.

In some aspects, the disclosure provides for a nucleic acid for use, wherein the nucleic acid encodes a HuPTMFabVEGFi, e.g., HuGlyFabVEGFi operatively linked to a promoter selected from the group consisting of: the CB7 promoter (a chicken R-actin promoter and CMV enhancer), cytomegalovirus (CMV) promoter, Rous sarcoma virus (RSV) promoter, MNT promoter, EF-1 alpha promoter, UB6 promoter, chicken beta-actin promoter, CAG promoter, RPE65 promoter and opsin promoter. In a specific embodiment, HuPTMFabVEGFi is operatively linked to the CB7 promoter.

In certain embodiments, provided herein are recombinant vectors that comprise one or more nucleic acids (e.g. polynucleotides). The nucleic acids may comprise DNA, RNA, or a combination of DNA and RNA. In certain embodiments, the DNA comprises one or more of the sequences selected from the group consisting of promoter sequences, the sequence encoding the therapeutic product of interest (the transgene, e.g., an anti-VEGF antigen-binding fragment), untranslated regions, and termination sequences. In certain embodiments, recombinant vectors provided herein comprise a promoter operably linked to the sequence encoding the therapeutic product of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

In certain embodiments, the recombinant vectors provided herein comprise modified mRNA encoding for the therapeutic product of interest (e.g., the transgene, for example, an anti-VEGF antigen-binding fragment moiety). In certain embodiments, provided herein is a modified mRNA encoding for an anti-VEGF antigen-binding fragment moiety. In certain embodiments, the recombinant vectors provided herein comprise a nucleotide sequence encoding for a therapeutic product that is an shRNA, siRNA, or miRNA.

In certain embodiments, the vectors provided herein comprise components that modulate protein delivery. In certain embodiments, the viral vectors provided herein comprise one or more signal peptides. Examples of signal peptides include, but is not limited to, VEGF-A signal peptide (SEQ ID NO: 5), fibulin-1 signal peptide (SEQ ID NO: 6), vitronectin signal peptide (SEQ ID NO: 7), complement Factor H signal peptide (SEQ ID NO: 8), opticin signal peptide (SEQ ID NO: 9), albumin signal peptide (SEQ ID NO: 22), chymotrypsinogen signal peptide (SEQ ID NO: 23), interleukin-2 signal peptide (SEQ ID NO: 24), and trypsinogen-2 signal peptide (SEQ ID NO: 25), mutant interleukin-2 signal peptide (SEQ ID NO: 55).

### 4.4.1 Viral Vectors

In some embodiments, the viral vectors provided herein are AAV based viral vectors. In preferred embodiments, the viral vectors provided herein are AAV8 based viral vectors. In certain embodiments, the AAV8 based viral vectors provided herein retain tropism for retinal cells. In certain embodiments, the AAV-based vectors provided herein encode the AAV rep gene (required for replication) and/or the AAV cap gene (required for synthesis of the capsid proteins). Multiple AAV serotypes have been identified. In certain embodiments, AAV-based vectors provided herein comprise components from one or more serotypes of AAV. In certain embodiments, AAV based vectors provided herein comprise capsid components from one or more of AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In preferred embodiments, AAV based vectors provided herein comprise components from one or more of AAV8, AAV9, AAV10, AAV11, or AAVrh10 serotypes. In certain embodiments, the recombinant viral vectors provided herein are altered such that they are replication-deficient in humans. In certain embodiments, the recombinant viral vectors are hybrid vectors, e.g., an AAV vector placed into a “helpless” adenoviral vector. In certain embodiments, provided herein are recombinant viral vectors comprising a viral capsid from a first virus and viral envelope proteins from a second virus. In specific embodiments, the second virus is vesicular stomatitis virus (VSV). In more specific embodiments, the envelope protein is VSV-G protein.

Provided in particular embodiments are AAV8 vectors comprising a viral genome comprising an expression cassette for expression of the transgene, under the control of regulatory elements and flanked by ITRs and a viral capsid that has the amino acid sequence of the AAV8 capsid protein or is at least 95%, 96%, 97%, 98%, 99% or 99.9% identical to the amino acid sequence of the AAV8 capsid protein (SEQ ID NO: 48) while retaining the biological function of the AAV8 capsid. In certain embodiments, the encoded AAV8 capsid has the sequence of SEQ ID NO: 48 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV8 capsid.

In certain embodiments, the AAV that is used in the methods described herein is Anc80 or Anc80L65, as described in Zinn et al., 2015, Cell Rep. 12(6): 1056-1068, which is incorporated by reference in its entirety. In certain embodiments, the AAV that is used in the methods described herein comprises one of the following amino acid insertions: LGETTRP or LALGETTRP, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is AAV.7m8, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is any AAV disclosed in U.S. Pat. No. 9,585,971, such as AAV.PHP.B. In certain embodiments, the AAV that is used in the methods described herein is an AAV disclosed in any of the following patents and patent applications, each of which is incorporated herein by reference in its entirety: U.S. Pat. Nos. 7,906,111; 8,524,446; 8,999,678; 8,628,966; 8,927,514; 8,734,809; 9,284,357; 9,409,953; 9,169,299; 9,193,956; 9,458,517; and 9,587,282 US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

AAV8-based viral vectors are used in certain of the methods described herein. Nucleic acid sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV8)-based viral vectors encoding a transgene (e.g., an anti-VEGF antigen-binding fragment). In specific embodiments, provided herein are AAV8-based viral vectors encoding an anti-VEGF antigen-binding fragment. In more specific embodiments, provided herein are AAV8-based viral vectors encoding ranibizumab.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the anti-VEGF antigen-binding fragment. The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approx. 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In a specific embodiment, a vector for use in the methods described herein is one that encodes an anti-VEGF antigen-binding fragment (e.g., ranibizumab) such that, upon introduction of the vector into a relevant cell (e.g., a retinal cell in vivo or in vitro), a glycosylated and or tyrosine sulfated variant of the anti-VEGF antigen-binding fragment is expressed by the cell. In a specific embodiment, the expressed anti-VEGF antigen-binding fragment comprises a glycosylation and/or tyrosine sulfation pattern.

### 4.4.2 Therapeutic Product or Transgenes

The therapeutic products can be, for example, therapeutic proteins (for example, antibodies), therapeutic RNAs (for example, shRNAs, siRNAs, and miRNAs), or therapeutic aptamers.

In certain embodiments, the disclosure provides a pharmaceutical composition comprising recombinant AAV encoding a transgene. In some embodiments, provided herein are rAAV viral vectors encoding an anti-VEGF Fab or anti-VEGF antibody. In some embodiments, provided herein are rAAV8-based viral vectors encoding an anti-VEGF Fab or anti-VEGF antibody. In some embodiments, provided herein are rAAV8-based viral vectors encoding ranibizumab. In some embodiments, provided herein are rAAV viral vectors encoding Iduronidase (IDUA). In some embodiments, provided herein are rAAV9-based viral vectors encoding IDUA. In some embodiments, provided herein are rAAV viral vectors encoding Iduronate 2-Sulfatase (IDS). In some embodiments, provided herein are rAAV9-based viral vectors encoding IDS. In some embodiments, provided herein are rAAV viral vectors encoding a low-density lipoprotein receptor (LDLR). In some embodiments, provided herein are rAAV8-based viral vectors encoding LDLR. In some embodiments, provided herein are rAAV viral vectors encoding tripeptidyl peptidase 1 (TPP1) protein. In some embodiments, provided herein are rAAV9-based viral vectors encoding TPP1. In some embodiments, provided herein are rAAV viral vectors encoding microdystrophin protein. In some embodiments, provided herein are rAAV8-based viral vectors encoding microdystrophin. In some embodiments, provided herein are rAAV9-based viral vectors encoding microdystrophin. In some embodiments, provided herein are rAAV viral vectors encoding anti-kallikrein (anti-pKal) protein. In some embodiments, provided herein are rAAV8-based or rAAV9-based viral vectors encoding lanadelumab Fab or full-length antibody. In some embodiments, provided herein are rAAV viral vectors encoding human-alpha-sarcoglycan-gamma-sarcoglycan. In some embodiments, provided herein are rAAV viral vectors encoding huFollistatin344. In some embodiments, provided herein are rAAV viral vectors encoding human-alpha-sarcoglycan-gamma-sarcoglycan. In some embodiments, provided herein are rAAV viral vectors encoding CLN2. In some embodiments, provided herein are rAAV viral vectors encoding CLN3. In some embodiments, provided herein are rAAV viral vectors encoding CLN6. In some embodiments, provided herein are rAAV8-based or rAAV9-based viral vectors encoding human-alpha-sarcoglycan-gamma-sarcoglycan. In some embodiments, provided herein are rAAV8-based or rAAV9-based viral vectors encoding huFollistatin344. In some embodiments, provided herein are rAAV8-based or rAAV9-based viral vectors encoding human-alpha-sarcoglycan-gamma-sarcoglycan. In some embodiments, provided herein are rAAV8-based or rAAV9-based viral vectors encoding CLN2. In some embodiments, provided herein are rAAV8-based or rAAV9-based viral vectors encoding CLN3. In some embodiments, provided herein are rAAV8-based or rAAV9-based viral vectors encoding CLN6.

In certain embodiments, the therapeutic product (e.g., transgene) is: (1) anti-human vascular endothelial growth factor (hVEGF) antibody or aptamer; (2) an anti-hVEGF antigen-binding fragment; (3) anti-hVEGF antigen-binding fragment is a Fab, F(ab′)2, or single chain variable fragment (scFv); (4) Palmitoyl-Protein Thioesterase 1 (PPT1); (5) Tripeptidyl-Peptidase 1 (TPP1); (6) Battenin (CLN3); and (7) CLN6 Transmembrane ER Protein (CLN6).

In certain embodiments, the disclosure provides a pharmaceutical composition comprising recombinant AAV encoding a transgene. In some embodiments, provided herein are rAAV viral vectors encoding an anti-VEGF Fab or anti-VEGF antibody. In some embodiments, provided herein are rAAV8-based viral vectors encoding an anti-VEGF Fab or anti-VEGF antibody. In more embodiments, provided herein are rAAV8-based viral vectors encoding ranibizumab. In some embodiments, provided herein are rAAV viral vectors encoding Iduronidase (IDUA). In some embodiments, provided herein are rAAV9-based viral vectors encoding IDUA. In some embodiments, provided herein are rAAV viral vectors encoding Iduronate 2-Sulfatase (IDS). In some embodiments, provided herein are rAAV9-based viral vectors encoding IDS. In some embodiments, provided herein are rAAV viral vectors encoding a low-density lipoprotein receptor (LDLR). In some embodiments, provided herein are rAAV8-based viral vectors encoding LDLR. In some embodiments, provided herein are rAAV viral vectors encoding tripeptidyl peptidase 1 (TPP1) protein. In some embodiments, provided herein are rAAV9-based viral vectors encoding TPP1. In some embodiments, provided herein are rAAV viral vectors encoding microdystrophin protein. In some embodiments, provided herein are rAAV8-based viral vectors encoding microdystrophin. In some embodiments, provided herein are rAAV9-based viral vectors encoding microdystrophin. In some embodiments, provided herein are rAAV viral vectors encoding anti-kallikrein (anti-pKal) protein. In some embodiments, provided herein are rAAV8-based or rAAV9-based viral vectors encoding lanadelumab Fab or full-length antibody. In some embodiments, provided herein are rAAV viral vectors encoding human-alpha-sarcoglycan-gamma-sarcoglycan. In some embodiments, provided herein are rAAV viral vectors encoding huFollistatin344. In some embodiments, provided herein are rAAV viral vectors encoding human-alpha-sarcoglycan-gamma-sarcoglycan. In some embodiments, provided herein are rAAV viral vectors encoding CLN2. In some embodiments, provided herein are rAAV viral vectors encoding CLN3. In some embodiments, provided herein are rAAV viral vectors encoding CLN6. In some embodiments, provided herein are rAAV8-based or rAAV9-based viral vectors encoding human-alpha-sarcoglycan-gamma-sarcoglycan. In some embodiments, provided herein are rAAV8-based or rAAV9-based viral vectors encoding huFollistatin344. In some embodiments, provided herein are rAAV8-based or rAAV9-based viral vectors encoding human-alpha-sarcoglycan-gamma-sarcoglycan. In some embodiments, provided herein are rAAV8-based or rAAV9-based viral vectors encoding CLN2. In some embodiments, provided herein are rAAV8-based or rAAV9-based viral vectors encoding CLN3. In some embodiments, provided herein are rAAV8-based or rAAV9-based viral vectors encoding CLN6.

In certain embodiments, the vectors provided herein can be used for (1) the pathology of the eye associated with Batten-CLN1 and the therapeutic product is Palmitoyl-Protein Thioesterase 1 (PPT1); (2) the pathology of the eye associated with Batten-CLN2 and the therapeutic product is Tripeptidyl-Peptidase 1 (TPP1); (3) the pathology of the eye associated with Batten-CLN3 and the therapeutic product is Battenin (CLN3); (4) the pathology of the eye associated with Batten-CLN6 and the therapeutic product is CLN6 Transmembrane ER Protein (CLN6); (5) the pathology of the eye associated with Batten-CLN7 and the therapeutic product is Major Facilitator Superfamily Domain Containing 8 (MFSD8); and (6) the pathology of the eye associated with Batten-CLN1 and the therapeutic product is Palmitoyl-Protein Thioesterase 1 (PPT1).

In some embodiments, the HuPTMFabVEGFi, e.g., HuGlyFabVEGFi encoded by the transgene can include, but is not limited to an antigen-binding fragment of an antibody that binds to VEGF, such as bevacizumab; an anti-VEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

In certain embodiments, the vectors provided herein encode an anti-VEGF antigen-binding fragment transgene. In specific embodiments, the anti-VEGF antigen-binding fragment transgene is controlled by appropriate expression control elements for expression in retinal cells: In certain embodiments, the anti-VEGF antigen-binding fragment transgene comprises bevacizumab Fab portion of the light and heavy chain cDNA sequences (SEQ ID Nos: 10 and 11, respectively). In certain embodiments, the anti-VEGF antigen-binding fragment transgene comprises ranibizumab light and heavy chain cDNA sequences (SEQ ID Nos: 12 and 13, respectively). In certain embodiments, the anti-VEGF antigen-binding fragment transgene encodes a bevacizumab Fab, comprising a light chain and a heavy chain of SEQ ID NOs: 3 and 4, respectively. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3. In certain embodiments, the anti-VEGF antigen-binding fragment transgene encodes an antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-VEGF antigen-binding fragment transgene encodes a hyperglycosylated ranibizumab, comprising a light chain and a heavy chain of SEQ ID NOs: 1 and 2, respectively. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In certain embodiments, the anti-VEGF antigen-binding fragment transgene encodes an antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2.

In certain embodiments, the anti-VEGF antigen-binding fragment transgene encodes a hyperglycosylated bevacizumab Fab, comprising a light chain and a heavy chain of SEQ ID NOs: 3 and 4, with one or more of the following mutations: L118N (heavy chain), E195N (light chain), or Q160N or Q160S (light chain). In certain embodiments, the anti-VEGF antigen-binding fragment transgene encodes a hyperglycosylated ranibizumab, comprising a light chain and a heavy chain of SEQ ID NOs: 1 and 2, with one or more of the following mutations: L118N (heavy chain), E195N (light chain), or Q160N or Q160S (light chain). The sequences of the antigen-binding fragment transgene cDNAs may be found, for example, in Table 1. In certain embodiments, the sequence of the antigen-binding fragment transgene cDNAs is obtained by replacing the signal sequence of SEQ ID NOs: 10 and 11 or SEQ ID NOs: 12 and 13 with one or more signal sequences.

In certain embodiments, the anti-VEGF antigen-binding fragment transgene encodes an antigen-binding fragment and comprises the nucleotide sequences of the six bevacizumab CDRs. In certain embodiments, the anti-VEGF antigen-binding fragment transgene encodes an antigen-binding fragment and comprises the nucleotide sequences of the six ranibizumab CDRs. In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21). In certain embodiments, the anti-VEGF antigen-binding fragment transgene encodes an antigen-binding fragment comprising a light chain variable region comprising light chain CDRs 1-3 of ranibizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19). In certain embodiments, the anti-VEGF antigen-binding fragment transgene encodes an antigen-binding fragment comprising a light chain variable region comprising light chain CDRs 1-3 of bevacizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21) and a light chain variable region comprising light chain CDRs 1-3 of ranibizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19) and a light chain variable region comprising light chain CDRs 1-3 of bevacizumab (SEQ ID NOs: 14-16).

In certain embodiments, the anti-VEGF antigen-binding fragment transgene encodes an antigen-binding fragment comprising a light chain variable region comprising light chain CDRs 1-3 of SEQ ID NOs: 14-16, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16, wherein the second amino acid residue of the light chain CDR3 (i.e., the second Q in QQYSTVPWTF (SEQ ID NO. 16)) is not acetylated. In a specific embodiment, the anti-VEGF antigen-binding fragment transgene encodes an antigen-binding fragment comprising a light chain variable region comprising light chain CDRs 1-3 of SEQ ID NOs: 14-16, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a heavy chain variable region comprising heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu), and wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated, and wherein the last amino acid residue of the heavy chain CDR1 (i.e., the N in GYDFTHYGMN (SEQ ID NO. 20)) is not acetylated. In a specific embodiment, the antigen-binding fragment comprises a heavy chain CDR1 of SEQ ID NO. 20, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated, and the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

TABLE 1
EXEMPLARY SEQUENCES

| SEQ | | |
|---|---|---|
| ID | | |
| NO: | Description | Sequence |
| 1 | Ranibizumab | DIQLTQSPSSLSASVGDRVTITCSASQDISNYLNWYQQKPGKAPKVLIYFTSSLH |
| | Fab Amino | SGVPSRFSGSGSGTDFTLTISSLQPEDFATYYCQQYSTVPWTFGQGTKVEIKRTV |
| | Acid Sequence | AAPSVFIFPPSDEQLKSGTASVVCLLNNFYPREAKVQWKVDNALQSGNSQESVTE |
| | (Light chain) | QDSKDSTYSLSSTLTLSKADYEKHKVYACEVTHQGLSSPVTKSENRGEC |
| 2 | Ranibizumab | EVQLVESGGGLVQPGGSLRLSCAASGYDFTHYGMNWVRQAPGKGLEWVGWINTYT |
| | Fab Amino | GEPTYAADFKRRFTFSLDTSKSTAYLQMNSLRAEDTAVYYCAKYPYYYGTSHWYF |
| | Acid Sequence | DVWGQGTLVTVSSASTKGPSVFPLAPSSKSTSGGTAALGCLVKDYFPEPVTVSWN |
| | (Heavy chain) | SGALTSGVHTFPAVLQSSGLYSLSSVVTVPSSSLGTQTYICNVNHKPSNTKVDKK |
| | | VEPKSCDKTHL |
| 3 | Bevacizumab | DIQMTQSPSSLSASVGDRVTITCSASQDISNYLNWYQQKPGKAPKVLIYFTSSLH |
| | Fab Amino | SGVPSRFSGSGSGTDFTLTISSLQPEDFATYYCQQYSTVPWTFGQGTKVEIKRTV |
| | Acid Sequence | AAPSVFIFPPSDEQLKSGTASVVCLLNNFYPREAKVQWKVDNALQSGNSQESVTE |
| | (Light chain) | QDSKDSTYSLSSTLTLSKADYEKHKVYACEVTHQGLSSPVTKSENRGEC |
| 4 | Bevacizumab | EVQLVESGGGLVQPGGSLRLSCAASGYTFTNYGMNWVRQAPGKGLEWVGWINTYT |
| | Fab Amino | GEPTYAADFKRRFTFSLDTSKSTAYLQMNSLRAEDTAVYYCAKYPHYYGSSHWYF |
| | Acid Sequence | DVWGQGTLVTVSSASTKGPSVFPLAPSSKSTSGGTAALGCLVKDYFPEPVTVSWN |
| | (Heavy chain) | SGALTSGVHTFPAVLQSSGLYSLSSVVTVPSSSLGTQTYICNVNHKPSNTKVDKK |
| | | VEPKSCDKTHL |
| 5 | VEGF-A signal | MNFLLSWVHW SLALLLYLHH AKWSQA |
| | peptide | |
| 6 | Fibulin-1 | MERAAPSRRV PLPLLLLGGL ALLAAGVDA |
| | signal peptide | |
| 7 | Vitronectin | MAPLRPLLIL ALLAWVALA |
| | signal peptide | |
| 8 | Complement | MRLLAKIICLMLWAICVA |
| | Factor H signal | |
| | peptide | |
| 9 | Opticin signal | MRLLAFLSLL ALVLQETGT |
| | peptide | |
| 10 | Bevacizumab | gctagcgcca ccatgggctg gtcctgcatc atcctgttcc tggtggccac |
| | CDNA | cgccaccggc gtgcactccg acatccagat gacccagtcc ccctcctccc |
| | (Light chain) | tgtccgcctc cgtgggcgac cgggtgacca tcacctgctc cgcctcccag |
| | | gacatctcca actacctgaa ctggtaccag cagaagcccg gcaaggcccc |
| | | caaggtgctg atctacttca cctcctccct gcactccggc gtgccctccc |
| | | ggttctccgg ctccggctcc ggcaccgact tcaccctgac catctcctcc |
| | | ctgcagcccg aggacttcgc cacctactac tgccagcagt actccaccgt |
| | | gccctggacc ttcggccagg gcaccaaggt ggagatcaag cggaccgtgg |
| | | ccgccccctc cgtgttcatc ttccccccct ccgacgagca gctgaagtcc |
| | | ggcaccgcct ccgtggtgtg cctgctgaac aacttctacc cccgggaggc |
| | | caaggtgcag tggaaggtgg acaacgccct gcagtccggc aactcccagg |
| | | agtccgtgac cgagcaggac tccaaggact ccacctactc cctgtcctcc |
| | | accctgaccc tgtccaaggc cgactacgag aagcacaagg tgtacgcctg |
| | | cgaggtgacc caccagggcc tgtcctcccc cgtgaccaag tccttcaacc |
| | | ggggcgagtg ctgagcggcc gcctcgag |
| 11 | Bevacizumab | gctagcgcca ccatgggctg gtcctgcatc atcctgttcc tggtggccac |
| | CDNA (Heavy | cgccaccggc gtgcactccg aggtgcagct ggtggagtcc ggcggcggcc |
| | chain) | tggtgcagcc cggcggctcc ctgcggctgt cctgcgccgc ctccggctac |
| | | accttcacca actacggcat gaactgggtg cggcaggccc ccggcaaggg |
| | | cctggagtgg gtgggctgga tcaacaccta caccggcgag cccacctacg |
| | | ccgccgactt caagcggcgg ttcaccttct ccctggacac ctccaagtcc |
| | | accgcctacc tgcagatgaa ctccctgcgg gccgaggaca ccgccgtgta |
| | | ctactgcgcc aagtaccccc actactacgg ctcctcccac tggtacttcg |
| | | acgtgtgggg ccagggcacc ctggtgaccg tgtcctccgc ctccaccaag |
| | | ggcccctccg tgttccccct ggccccctcc tccaagtcca cctccggcgg |
| | | caccgccgcc ctgggctgcc tggtgaagga ctacttcccc gagcccgtga |
| | | ccgtgtcctg gaactccggc gccctgacct ccggcgtgca caccttcccc |
| | | gccgtgctgc agtcctccgg cctgtactcc ctgtcctccg tggtgaccgt |
| | | gccctcctcc tccctgggca cccagaccta catctgcaac gtgaaccaca |
| | | agccctccaa caccaaggtg gacaagaagg tggagcccaa gtcctgcgac |
| | | aagacccaca cctgcccccc ctgccccgcc cccgagctgc tggggggccc |
| | | ctccgtgttc ctgttccccc ccaagcccaa ggacaccctg atgatctccc |
| | | ggacccccga ggtgacctgc gtggtggtgg acgtgtccca cgaggacccc |
| | | gaggtgaagt tcaactggta cgtggacggc gtggaggtgc acaacgccaa |
| | | gaccaagccc cgggaggagc agtacaactc cacctaccgg gtggtgtccg |
| | | tgctgaccgt gctgcaccag gactggctga acggcaagga gtacaagtgc |
| | | aaggtgtcca acaaggccct gcccgccccc atcgagaaga ccatctccaa |
| | | ggccaagggc cagccccggg agccccaggt gtacaccctg cccccctccc |
| | | gggaggagat gaccaagaac caggtgtccc tgacctgcct ggtgaagggc |
| | | ttctacccct ccgacatcgc cgtggagtgg gagtccaacg gccagcccga |
| | | gaacaactac aagaccaccc cccccgtgct ggactccgac ggctccttct |
| | | tcctgtactc caagctgacc gtggacaagt cccggtggca gcagggcaac |
| | | gtgttctcct gctccgtgat gcacgaggcc ctgcacaacc actacaccca |
| | | gaagtccctg tccctgtccc ccggcaagtg agcggccgcc |
| 12 | Ranibizumab | gagctccatg gagtttttca aaaagacggc acttgccgca ctggttatgg |
| | cDNA (Light | gttttagtgg tgcagcattg gccgatatcc agctgaccca gtagcgcaag |
| | chain | agcctgagcg caagcgttgg tgatcgtgtt accattacct ccgggtaaag |
| | comprising a | ccaggatatt agcaattatc tgaattggta tcagcagaaa cggtgttccg |
| | signal | caccgaaagt tctgatttat tttaccagca gcctgcatag tgaccattag |
| | sequence) | agccgtttta gcggtagcgg tagtggcacc gattttaccc cagtatagca |
| | | cagcctgcag ccggaagatt ttgcaaccta ttattgtcag taaacgtacc |
| | | ccgttccgtg gacctttggt cagggcacca aagttgaaat aacagctgaa |
| | | gttgcagcac cgagcgtttt tatttttccg cctagtgatg tatccgcgtg |
| | | aagcggcacc gcaagcgttg tttgtctgct gaataatttt cggtaatagc |
| | | aagcaaaagt gcagtggaaa gttgataatg cactgcagag atagcctgag |
| | | caagaaagcg ttaccgaaca ggatagcaaa gatagcacct aaagtgtatg |
| | | cagcaccctg accctgagca aagcagatta tgaaaaacac caaaagtttt |
| | | cctgcgaagt tacccatcag ggtctgagca gtccggttac gagcccgagc |
| | | aatcgtggcg aatgctaata gaagcttggt acc |
| 13 | Ranibizumab | gagctcatat gaaatacctg ctgccgaccg ctgctgctgg tctgctgctc |
| | cDNA (Heavy | ctcgctgccc agccggcgat ggccgaagtt cagctggttg aaagcggtgg |
| | chain | tggtctggtt cagcctggtg gtagcctgcg tctgagctgt gcagcaagcg |
| | comprising a | gttatgattt tacccattat ggtatgaatt gggttcgtca ggcaccgggt |
| | signal | aaaggtctgg aatgggttgg ttggattaat acctataccg gtgaaccgac |
| | sequence) | ctatgcagca gattttaaac gtcgttttac ctttagcctg gataccagca |
| | | aaagcaccgc atatctgcag atgaatagcc tgcgtgcaga agataccgca |
| | | gtttattatt gtgccaaata tccgtattac tatggcacca gccactggta |
| | | tttcgatgtt tggggtcagg gcaccctggt taccgttagc agcgcaagca |
| | | ccaaaggtcc gagcgttttt ccgctggcac cgagcagcaa aagtaccagc |
| | | ggtggcacag cagcactggg ttgtctggtt aaagattatt ttccggaacc |
| | | ggttaccgtg agctggaata gcggtgcact gaccagcggt gttcatacct |
| | | ttccggcagt tctgcagagc agcggtctgt atagcctgag cagcgttgtt |
| | | accgttccga gcagcagcct gggcacccag acctatattt gtaatgttaa |
| | | tcataaaccg agcaatacca aagtggataa aaaagttgag ccgaaaagct |
| | | gcgataaaac ccatctgtaa tagggtacc |
| 14 | Bevacizumab | SASQDISNYLN |
| | and | |
| | Ranibizumab | |
| | Light Chain | |
| | CDR1 | |
| 15 | Bevacizumab | FTSSLHS |
| | and | |
| | Ranibizumab | |
| | Light Chain | |
| | CDR2 | |
| 16 | Bevacizumab | QQYSTVPWT |
| | and | |
| | Ranibizumab | |
| | Light Chain | |
| | CDR3 | |
| 17 | Bevacizumab | GYTFTNYGMN |
| | Heavy Chain | |
| | CDR1 | |
| 18 | Bevacizumab | WINTYTGEPTYAADFKR |
| | and | |
| | Ranibizumab | |
| | Heavy Chain | |
| | CDR2 | |
| 19 | Bevacizumab | YPHYYGSSHWYFDV |
| | Heavy Chain | |
| | CDR3 | |
| 20 | Ranibizumab | GYDFTHYGMN |
| | Heavy Chain | |
| | CDR1 | |
| 21 | Ranibizumab | YPYYYGTSHWYFDV |
| | Heavy Chain | |
| | CDR3 | |
| 22 | Albumin signal | MKWVTFISLLFLESSAYS |
| | peptide | |
| 23 | Chymotrypsinogen | MAFLWLLSCWALLGTTFG |
| | signal | |
| | peptide | |
| 24 | Interleukin-2 | MYRMQLLSCIALILALVINS |
| | signal peptide | |
| 25 | Trypsinogen-2 | MNLLLILTFVAAAVA |
| | signal peptide | |
| 26 | F2A site | LLNFDLLKLAGDVESNPGP |
| 27 | T2A site | (GSG)EGRGSLLTCGDVEENPGP |
| 28 | P2A site | (GSG)ATNFSLLKQAGDVEENPGP |
| 29 | E2A site | (GSG)QCTNYALLKLAGDVESNPGP |
| 30 | F2A site | (GSG)VKQTLNFDLLKLAGDVESNPGP |
| 31 | Furin linker | RKRR |
| 32 | Furin linker | RRRR |
| 33 | Furin linker | RRKR |
| 34 | Furin linker | RKKR |
| 35 | Furin linker | R-X-K/R-R |
| 36 | Furin linker | RXKR |
| 37 | Furin linker | RXRR |
| 38 | Ranibizumab | MDIQLTQSPSSLSASVGDRVTITCSASQDISNYLNWYQQKPGKAPKVLIYFTSSL |
| | Fab amino acid | HSGVPSRFSGSGSGTDFTLTISSLQPEDFATYYCQQYSTVPWTFGQGTKVEIKRT |
| | sequence (Light | VAAPSVFIFPPSDEQLKSGTASVVCLLNNFYPREAKVQWKVDNALQSGNSQESVT |
| | chain) | EQDSKDSTYSLSSTLTLSKADYEKHKVYACEVTHQGLSSPVTKSENRGEC |
| 39 | Ranibizumab | MEVQLVESGGGLVQPGGSLRLSCAASGYDFTHYGMNWVRQAPGKGLEWVGWINTY |
| | Fab amino acid | TGEPTYAADFKRRFTFSLDTSKSTAYLQMNSLRAEDTAVYYCAKYPYYYGTSHWY |
| | sequence | FDVWGQGTLVTVSSASTKGPSVFPLAPSSKSTSGGTAALGCLVKDYFPEPVTVSW |
| | (Heavy chain) | NSGALTSGVHTFPAVLQSSGLYSLSSVVTVPSSSLGTQTYICNVNHKPSNTKVDK |
| | | KVEPKSCDKTHLRKRR |
| 40 | Ranibizumab | MEVQLVESGGGLVQPGGSLRLSCAASGYDFTHYGMNWVRQAPGKGLEWVGWINTY |
| | Fab amino acid | TGEPTYAADFKRRFTFSLDTSKSTAYLQMNSLRAEDTAVYYCAKYPYYYGTSHWY |
| | sequence | FDVWGQGTLVTVSSASTKGPSVFPLAPSSKSTSGGTAALGCLVKDYFPEPVTVSW |
| | (Heavy chain) | NSGALTSGVHTFPAVLQSSGLYSLSSVVTVPSSSLGTQTYICNVNHKPSNTKVDK |
| | | KVEPKSCDKTHL |
| 41 | AAV1 | MAADGYLPDWLEDNLSEGIREWWDLKPGAPKPKANQQKQDDGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPLGLVEEGAKTAPGKKRPVEQSPQEPDSSSGIGKTGQQ |
| | | PAKKRLNFGQTGDSESVPDPQPLGEPPATPAAVGPTTMASGGGAPMADNNEGADG |
| | | VGNASGNWHCDSTWLGDRVITTSTRTWALPTYNNHLYKQISSASTGASNDNHYFG |
| | | YSTPWGYFDFNRFHCHFSPRDWQRLINNNWGFRPKRLNFKLFNIQVKEVTTNDGV |
| | | TTIANNLTSTVQVESDSEYQLPYVLGSAHQGCLPPFPADVFMIPQYGYLTLNNGS |
| | | QAVGRSSFYCLEYFPSQMLRTGNNFTFSYTFEEVPFHSSYAHSQSLDRLMNPLID |
| | | QYLYYLNRTQNQSGSAQNKDLLFSRGSPAGMSVQPKNWLPGPCYRQQRVSKTKTD |
| | | NNNSNFTWTGASKYNLNGRESIINPGTAMASHKDDEDKFFPMSGVMIFGKESAGA |
| | | SNTALDNVMITDEEEIKATNPVATERFGTVAVNFQSSSTDPATGDVHAMGALPGM |
| | | VWQDRDVYLQGPIWAKIPHTDGHFHPSPLMGGFGLKNPPPQILIKNTPVPANPPA |
| | | EFSATKFASFITQYSTGQVSVEIEWELQKENSKRWNPEVQYTSNYAKSANVDFTV |
| | | DNNGLYTEPRPIGTRYLTRPL |
| 42 | AAV2 | MAADGYLPDWLEDTLSEGIRQWWKLKPGPPPPKPAERHKDDSRGLVLPGYKYLGP |
| | | FNGLDKGEPVNEADAAALEHDKAYDRQLDSGDNPYLKYNHADAEFQERLKEDTSF |
| | | GGNLGRAVFQAKKRVLEPLGLVEEPVKTAPGKKRPVEHSPVEPDSSSGTGKAGQQ |
| | | PARKRLNFGQTGDADSVPDPQPLGQPPAAPSGLGTNTMATGSGAPMADNNEGADG |
| | | VGNSSGNWHCDSTWMGDRVITTSTRTWALPTYNNHLYKQISSQSGASNDNHYFGY |
| | | STPWGYFDFNRFHCHFSPRDWQRLINNNWGFRPKRLNFKLFNIQVKEVTQNDGTT |
| | | TIANNLTSTVQVFTDSEYQLPYVLGSAHQGCLPPFPADVFMVPQYGYLTLNNGSQ |
| | | AVGRSSFYCLEYFPSQMLRTGNNFTFSYTFEDVPFHSSYAHSQSLDRLMNPLIDQ |
| | | YLYYLSRTNTPSGTTTQSRLQFSQAGASDIRDQSRNWLPGPCYRQQRVSKTSADN |
| | | NNSEYSWTGATKYHLNGRDSLVNPGPAMASHKDDEEKFFPQSGVLIFGKQGSEKT |
| | | NVDIEKVMITDEEEIRTTNPVATEQYGSVSTNLQRGNRQAATADVNTQGVLPGMV |
| | | WQDRDVYLQGPIWAKIPHTDGHFHPSPLMGGFGLKHPPPQILIKNTPVPANPSTT |
| | | FSAAKFASFITQYSTGQVSVEIEWELQKENSKRWNPEIQYTSNYNKSVNVDFTVD |
| | | TNGVYSEPRPIGTRYLTRNL |
| 43 | AAV3-3 | MAADGYLPDWLEDNLSEGIREWWALKPGVPQPKANQQHQDNRRGLVLPGYKYLGP |
| | | GNGLDKGEPVNEADAAALEHDKAYDQQLKAGDNPYLKYNHADAEFQERLQEDTSF |
| | | GGNLGRAVFQAKKRILEPLGLVEEAAKTAPGKKGAVDQSPQEPDSSSGVGKSGKQ |
| | | PARKRLNFGQTGDSESVPDPQPLGEPPAAPTSLGSNTMASGGGAPMADNNEGADG |
| | | VGNSSGNWHCDSQWLGDRVITTSTRTWALPTYNNHLYKQISSQSGASNDNHYFGY |
| | | STPWGYFDFNRFHCHFSPRDWQRLINNNWGFRPKKLSFKLFNIQVRGVTQNDGTT |
| | | TIANNLTSTVQVFTDSEYQLPYVLGSAHQGCLPPFPADVFMVPQYGYLTLNNGSQ |
| | | AVGRSSFYCLEYFPSQMLRTGNNFQFSYTFEDVPFHSSYAHSQSLDRLMNPLIDQ |
| | | YLYYLNRTQGTTSGTTNQSRLLFSQAGPQSMSLQARNWLPGPCYRQQRLSKTAND |
| | | NNNSNFPWTAASKYHLNGRDSLVNPGPAMASHKDDEEKFFPMHGNLIFGKEGTTA |
| | | SNAELDNVMITDEEEIRTTNPVATEQYGTVANNLQSSNTAPTTGTVNHQGALPGM |
| | | VWQDRDVYLQGPIWAKIPHTDGHFHPSPLMGGFGLKHPPPQIMIKNTPVPANPPT |
| | | TFSPAKFASFITQYSTGQVSVEIEWELQKENSKRWNPEIQYTSNYNKSVNVDFTV |
| | | DTNGVYSEPRPIGTRYLTRNL |
| 44 | AAV4-4 | MTDGYLPDWLEDNLSEGVREWWALQPGAPKPKANQQHQDNARGLVLPGYKYLGPG |
| | | NGLDKGEPVNAADAAALEHDKAYDQQLKAGDNPYLKYNHADAEFQQRLQGDTSFG |
| | | GNLGRAVFQAKKRVLEPLGLVEQAGETAPGKKRPLIESPQQPDSSTGIGKKGKQP |
| | | AKKKLVFEDETGAGDGPPEGSTSGAMSDDSEMRAAAGGAAVEGGQGADGVGNASG |
| | | DWHCDSTWSEGHVTTTSTRTWVLPTYNNHLYKRLGESLQSNTYNGFSTPWGYFDF |
| | | NRFHCHFSPRDWQRLINNNWGMRPKAMRVKIFNIQVKEVTTSNGETTVANNLTST |
| | | VQIFADSSYELPYVMDAGQEGSLPPFPNDVFMVPQYGYCGLVTGNTSQQQTDRNA |
| | | FYCLEYFPSQMLRTGNNFEITYSFEKVPFHSMYAHSQSLDRLMNPLIDQYLWGLQ |
| | | STTTGTTLNAGTATTNFTKLRPTNFSNFKKNWLPGPSIKQQGFSKTANQNYKIPA |
| | | TGSDSLIKYETHSTLDGRWSALTPGPPMATAGPADSKFSNSQLIFAGPKQNGNTA |
| | | TVPGTLIFTSEEELAATNATDTDMWGNLPGGDQSNSNLPTVDRLTALGAVPGMVW |
| | | QNRDIYYQGPIWAKIPHTDGHFHPSPLIGGFGLKHPPPQIFIKNTPVPANPATTE |
| | | SSTPVNSFITQYSTGQVSVQIDWEIQKERSKRWNPEVQFTSNYGQQNSLLWAPDA |
| | | AGKYTEPRAIGTRYLTHHL |
| 45 | AAV5 | MSFVDHPPDWLEEVGEGLREFLGLEAGPPKPKPNQQHQDQARGLVLPGYNYLGPG |
| | | NGLDRGEPVNRADEVAREHDISYNEQLEAGDNPYLKYNHADAEFQEKLADDTSFG |
| | | GNLGKAVFQAKKRVLEPFGLVEEGAKTAPTGKRIDDHFPKRKKARTEEDSKPSTS |
| | | SDAEAGPSGSQQLQIPAQPASSLGADTMSAGGGGPLGDNNQGADGVGNASGDWHC |
| | | DSTWMGDRVVTKSTRTWVLPSYNNHQYREIKSGSVDGSNANAYFGYSTPWGYFDF |
| | | NRFHSHWSPRDWQRLINNYWGFRPRSLRVKIFNIQVKEVTVQDSTTTIANNLTST |
| | | VQVFTDDDYQLPYVVGNGTEGCLPAFPPQVFTLPQYGYATLNRDNTENPTERSSF |
| | | FCLEYFPSKMLRTGNNFEFTYNFEEVPFHSSFAPSQNLFKLANPLVDQYLYRFVS |
| | | TNNTGGVQFNKNLAGRYANTYKNWFPGPMGRTQGWNLGSGVNRASVSAFATTNRM |
| | | ELEGASYQVPPQPNGMTNNLQGSNTYALENTMIFNSQPANPGTTATYLEGNMLIT |
| | | SESETQPVNRVAYNVGGQMATNNQSSTTAPATGTYNLQEIVPGSVWMERDVYLQG |
| | | PIWAKIPETGAHFHPSPAMGGFGLKHPPPMMLIKNTPVPGNITSFSDVPVSSFIT |
| | | QYSTGQVTVEMEWELKKENSKRWNPEIQYTNNYNDPQFVDFAPDSTGEYRTTRPI |
| | | GTRYLTRPL |
| 46 | AAV6 | MAADGYLPDWLEDNLSEGIREWWDLKPGAPKPKANQQKQDDGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPFGLVEEGAKTAPGKKRPVEQSPQEPDSSSGIGKTGQQ |
| | | PAKKRLNFGQTGDSESVPDPQPLGEPPATPAAVGPTTMASGGGAPMADNNEGADG |
| | | VGNASGNWHCDSTWLGDRVITTSTRTWALPTYNNHLYKQISSASTGASNDNHYFG |
| | | YSTPWGYFDFNRFHCHFSPRDWQRLINNNWGFRPKRLNFKLFNIQVKEVTTNDGV |
| | | TTIANNLTSTVQVFSDSEYQLPYVLGSAHQGCLPPFPADVFMIPQYGYLTLNNGS |
| | | QAVGRSSFYCLEYFPSQMLRTGNNFTFSYTFEDVPFHSSYAHSQSLDRLMNPLID |
| | | QYLYYLNRTQNQSGSAQNKDLLFSRGSPAGMSVQPKNWLPGPCYRQQRVSKTKTD |
| | | NNNSNFTWTGASKYNLNGRESIINPGTAMASHKDDKDKFFPMSGVMIFGKESAGA |
| | | SNTALDNVMITDEEEIKATNPVATERFGTVAVNLQSSSTDPATGDVHVMGALPGM |
| | | VWQDRDVYLQGPIWAKIPHTDGHFHPSPLMGGFGLKHPPPQILIKNTPVPANPPA |
| | | EFSATKFASFITQYSTGQVSVEIEWELQKENSKRWNPEVQYTSNYAKSANVDFTV |
| | | DNNGLYTEPRPIGTRYLTRPL |
| 47 | AAV7 | MAADGYLPDWLEDNLSEGIREWWDLKPGAPKPKANQQKQDNGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPLGLVEEGAKTAPAKKRPVEPSPQRSPDSSTGIGKKGQ |
| | | QPARKRLNFGQTGDSESVPDPQPLGEPPAAPSSVGSGTVAAGGGAPMADNNEGAD |
| | | GVGNASGNWHCDSTWLGDRVITTSTRTWALPTYNNHLYKQISSETAGSTNDNTYF |
| | | GYSTPWGYFDFNRFHCHFSPRDWQRLINNNWGFRPKKLRFKLFNIQVKEVTTNDG |
| | | VTTIANNLTSTIQVFSDSEYQLPYVLGSAHQGCLPPFPADVEMIPQYGYLTLNNG |
| | | SQSVGRSSFYCLEYFPSQMLRTGNNFEFSYSFEDVPFHSSYAHSQSLDRLMNPLI |
| | | DQYLYYLARTQSNPGGTAGNRELQFYQGGPSTMAEQAKNWLPGPCFRQQRVSKTL |
| | | DQNNNSNFAWTGATKYHLNGRNSLVNPGVAMATHKDDEDRFFPSSGVLIFGKTGA |
| | | TNKTTLENVLMTNEEEIRPTNPVATEEYGIVSSNLQAANTAAQTQVVNNQGALPG |
| | | MVWQNRDVYLQGPIWAKIPHTDGNFHPSPLMGGFGLKHPPPQILIKNTPVPANPP |
| | | EVFTPAKFASFITQYSTGQVSVEIEWELQKENSKRWNPEIQYTSNFEKQTGVDFA |
| | | VDSQGVYSEPRPIGTRYLTRNL |
| 48 | AAV8 | MAADGYLPDWLEDNLSEGIREWWALKPGAPKPKANQQKQDDGRGLVLPGYKYLGP |
| | | FNGLDKGEPVNAADAAALEHDKAYDQQLQAGDNPYLRYNHADAEFQERLQEDTSF |
| | | GGNLGRAVFQAKKRVLEPLGLVEEGAKTAPGKKRPVEPSPQRSPDSSTGIGKKGQ |
| | | QPARKRLNFGQTGDSESVPDPQPLGEPPAAPSGVGPNTMAAGGGAPMADNNEGAD |
| | | GVGSSSGNWHCDSTWLGDRVITTSTRTWALPTYNNHLYKQISNGTSGGATNDNTY |
| | | FGYSTPWGYFDENRFHCHFSPRDWQRLINNNWGFRPKRLSFKLFNIQVKEVTQNE |
| | | GTKTIANNLTSTIQVFTDSEYQLPYVLGSAHQGCLPPFPADVFMIPQYGYLTLNN |
| | | GSQAVGRSSFYCLEYFPSQMLRTGNNFQFTYTFEDVPFHSSYAHSQSLDRLMNPL |
| | | IDQYLYYLSRTQTTGGTANTQTLGFSQGGPNTMANQAKNWLPGPCYRQQRVSTTT |
| | | GQNNNSNFAWTAGTKYHLNGRNSLANPGIAMATHKDDEERFFPSNGILIFGKQNA |
| | | ARDNADYSDVMLTSEEEIKTTNPVATEEYGIVADNLQQQNTAPQIGTVNSQGALP |
| | | GMVWQNRDVYLQGPIWAKIPHTDGNFHPSPLMGGFGLKHPPPQILIKNTPVPADP |
| | | PTTFNQSKLNSFITQYSTGQVSVEIEWELQKENSKRWNPEIQYTSNYYKSTSVDF |
| | | AVNTEGVYSEPRPIGTRYLTRNL |
| 49 | hu31 | MAADGYLPDWLEDTLSEGIRQWWKLKPGPPPPKPAERHKDDSRGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGSQ |
| | | PAKKKLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDFNRFHCHFSPRDWQRLINNNWGFRPKRLNFKLFNIQVKEVTDNNG |
| | | VKTIANNLTSTVQVFTDSDYQLPYVLGSAHEGCLPPFPADVFMIPQYGYLTLNDG |
| | | GQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | STEGVYSEPRPIGTRYLTRNL |
| 50 | hu32 | MAADGYLPDWLEDTLSEGIRQWWKLKPGPPPPKPAERHKDDSRGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGSQ |
| | | PAKKKLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDFNRFHCHFSPRDWQRLINNNWGFRPKRLNFKLFNIQVKEVTDNNG |
| | | VKTIANNLTSTVQVFTDSDYQLPYVLGSAHEGCLPPFPADVFMIPQYGYLTLNDG |
| | | SQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | NTEGVYSEPRPIGTRYLTRNL |
| 51 | AAV9 | MAADGYLPDWLEDNLSEGIREWWALKPGAPQPKANQQHQDNARGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGAQ |
| | | PAKKRLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDFNRFHCHFSPRDWQRLINNNWGFRPKRLNFKLFNIQVKEVTDNNG |
| | | VKTIANNLTSTVQVFTDSDYQLPYVLGSAHEGCLPPFPADVFMIPQYGYLTLNDG |
| | | SQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | NTEGVYSEPRPIGTRYLTRNL |
| 52 | Vascular | MNFLLSWVHWSLALLLYLHHAKWSQAAPMAEGGGQNHHEVVKFMDVYQRSYCHPI |
| | endothelial | ETLVDIFQEYPDEIEYIFKPSCVPLMRCGGCCNDEGLECVPTEESNITMQIMRIK |
| | growth factor | PHQGQHIGEMSFLQHNKCECRPKKDRARQENPCGPCSERRKHLFVQDPQTCKCSC |
| | (vegf) | KNTDSRCKARQLELNERTCRCDKPRR |
| | Caa44447.1 | |
| 53 | Palmitoyl- | MASPGCLWLLAVALLPWTCASRALQHLDPPAPLPLVIWHGMGDSCCNPLSMGAIK |
| | protein | KMVEKKIPGIYVLSLEIGKTLMEDVENSFFLNVNSQVTTVCQALAKDPKLQQGYN |
| | thioesterase 1 | AMGFSQGGQFLRAVAQRCPSPPMINLISVGGQHQGVFGLPRCPGESSHICDFIRK |
| | (ppt1) | TLNAGAYSKVVQERLVQAEYWHDPIKEDVYRNHSIFLADINQERGINESYKKNLM |
| | Aah08426.1 | ALKKFVMVKFLNDSIVDPVDSEWFGFYRSGQAKETIPLQETSLYTQDRLGLKEMD |
| | | NAGQLVFLATEGDHLQLSEEWFYAHIIPFLG |
| 54 | Tripeptidyl- | MGLQACLLGLFALILSGKCSYSPEPDQRRTLPPGWVSLGRADPEEELSLTFALRQ |
| | peptidase 1 | QNVERLSELVQAVSDPSSPQYGKYLTLENVADLVRPSPLTLHTVQKWLLAAGAQK |
| | (tpp1) | CHSVITQDFLTCWLSIRQAELLLPGAEFHHYVGGPTETHVVRSPHPYQLPQALAP |
| | Np_000382.3 | HVDFVGGLHRFPPTSSLRQRPEPQVTGTVGLHLGVTPSVIRKRYNLTSQDVGSGT |
| | | SNNSQACAQFLEQYFHDSDLAQFMRLFGGNFAHQASVARVVGQQGRGRAGIEASL |
| | | DVQYLMSAGANISTWVYSSPGRHEGQEPFLQWLMLLSNESALPHVHTVSYGDDED |
| | | SLSSAYIQRVNTELMKAAARGLTLLFASGDSGAGCWSVSGRHQFRPTFPASSPYV |
| | | TTVGGTSFQEPFLITNEIVDYISGGGFSNVFPRPSYQEEAVTKFLSSSPHLPPSS |
| | | YFNASGRAYPDVAALSDGYWVVSNRVPIPWVSGTSASTPVFGGILSLINEHRILS |
| | | GRPPLGFLNPRLYQQHGAGLFDVTRGCHESCLDEEVEGQGFCSGPGWDPVTGWGT |
| | | PNFPALLKTLLNP |
| 55 | Mutant | MYRMQLLLLIALSLALVINS |
| | interleukin-2 | |
| | signal peptide | |

### 4.5 Diseases

The pharmaceutical composition or the reference pharmaceutical composition provided herein (e.g., Section 4.1) can be administered to a subject diagnosed with nAMD (wet AMD), dry AMD, retinal vein occlusion (RVO), diabetic macular edema (DME), diabetic retinopathy (DR), or Batten disease.

In some embodiments, disclosed herein are methods of treating a subject diagnosed with nAMD (wet AMD), dry AMD, retinal vein occlusion (RVO), diabetic macular edema (DME), diabetic retinopathy (DR), or Batten by administering to the subject a therapeutically effective amount of the pharmaceutical composition by suprachoroidal injection (for example, via a suprachoroidal drug delivery device such as a microinjector with a microneedle).

In some embodiments, a pharmaceutical composition containing about 2.5×10¹¹ GC/eye, about 5×10¹¹ GC/eye, or about 1.5×10¹² GC/eye of Construct II of a pharmaceutical composition comprising 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL, 4% w/v sucrose, and optionally a surfactant is administered to a patient via suprachoroidal administration. In some embodiments, the patient has diabetic retinopathy.

In some embodiments, a pharmaceutical composition containing about 2.5×10¹¹ GC/eye, about 5×10¹¹ GC/eye, or about 1.5×10¹² GC/eye of Construct II of a pharmaceutical composition comprising 10% w/v sucrose is administered to a patient via suprachoroidal administration. In some embodiments, the patient has diabetic retinopathy. In some embodiments, the pharmaceutical composition has a tonicity/osmolality equal to or greater than 240 mOsm/kg.

In some aspects, disclosed herein are pharmaceutical compositions suitable for, or methods of, treating a subject diagnosed with mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis, or kallikrein-related disease comprising administering to the subject a therapeutically effective amount of the pharmaceutical composition. In some embodiments, the pharmaceutical composition is administered in the SCS.

In some embodiments, the pharmaceutical composition or the reference pharmaceutical composition provided herein (e.g., Section 4.1) can be administered to a subject diagnosed with (1) Batten-CLN2 and the therapeutic product is Tripeptidyl-Peptidase 1 (TPP1); (2) Usher's-Type 1 and the therapeutic product is Myosin VIIA (MYO7A); (3) Usher's-Type 1 and the therapeutic product is Cadherin Related 23 (CDH23); (4) Usher's-Type 2 and the therapeutic product is Protocadherin Related 15 (PCDH15); (5) Usher's-Type 2 and the therapeutic product is Usherin (USH2A); (6) Usher's-Type 3 and the therapeutic product is Clarin 1 (CLRN1); (7) Stargardt's and the therapeutic product is ATP Binding Cassette Subfamily A Member 4 (ABCA4); (8) Stargardt's and the therapeutic product is ELOVL Fatty Acid Elongase 4 (ELOVL4); (9) red-green color blindness and the therapeutic product is L opsin (OPN1LW); (10) red-green color blindness and the therapeutic product is M opsin (OPN1MW); (11) blue cone monochromacy and the therapeutic product is M opsin (OPN1MW); (12) Leber congenital amaurosis-1 (LCA 1) and the therapeutic product is Guanylate Cyclase 2D, Retinal (GUCY2D); (13) Leber congenital amaurosis-2 (LCA 2) and the therapeutic product is Retinoid Isomerohydrolase RPE65 (RPE65); (14) Leber congenital amaurosis-4 (LCA 4) and the therapeutic product is Aryl Hydrocarbon Receptor Interacting Protein Like 1 (AIPL1); (15) Leber congenital amaurosis-7 (LCA 7) and the therapeutic product is Cone-Rod Homeobox (CRX); (16) Leber congenital amaurosis-8 (LCA 8) and the therapeutic product is Crumbs Cell Polarity Complex Component 1 (CRB1); (17) Leber congenital amaurosis-9 (LCA 9) and the therapeutic product is Nicotinamide Nucleotide Adenylyltransferase 1 (NMNAT1); (18) Leber congenital amaurosis-10 (LCA 10) and the therapeutic product is Centrosomal Protein 290 (CEP290); (19) Leber congenital amaurosis-11 (LCA 11) and the therapeutic product is Inosine Monophosphate Dehydrogenase 1 (IMPDH1); (20) Leber congenital amaurosis-15 (LCA 15) and the therapeutic product is Tubby Like Protein 1 (TULP1); (21) LHON and the therapeutic product is Mitochondrially Encoded NADH Dehydrogenase 4 (MT-ND4); (22) LHON and the therapeutic product is Mitochondrially Encoded NADH Dehydrogenase 6 (MT-ND6); (23) choroideremia and the therapeutic product is Rab Escort Protein 1 (CHM); (24) X-linked retinoschisis (XLRS) and the therapeutic product is Retinoschisin (RS1); (25) Bardet-Biedl syndrome 1 and the therapeutic product is Bardet-Biedl Syndrome 1 (BBS1); (26) Bardet-Biedl syndrome 6 and the therapeutic product is McKusick-Kaufman Syndrome (MKKS); (27) Bardet-Biedl syndrome 10 and the therapeutic product is Bardet-Biedl Syndrome 10 (BBS10); (28) cone dystrophy and the therapeutic product is Guanylate Cyclase Activator 1A (GUCA1A); (29) optic atrophy and the therapeutic product is OPA1 Mitochondrial Dynamin Like GTPase (OPA1); (30) retinitis pigmentosa 1 and the therapeutic product is RP1 Axonemal Microtubule Associated (RP1); (31) retinitis pigmentosa 2 and the therapeutic product is RP2 Activator of ARL3 GTPase (RP2); (32) retinitis pigmentosa 7 and the therapeutic product is Peripherin 2 (PRPH2); (33) retinitis pigmentosa 11 and the therapeutic product is Pre-mRNA Processing Factor 31 (PRPF31); (34) retinitis pigmentosa 13 and the therapeutic product is Pre-mRNA Processing Factor 8 (PRPF8); (35) retinitis pigmentosa 37 and the therapeutic product is Nuclear Receptor Subfamily 2 Group E Member 3 (NR2E3); (36) retinitis pigmentosa 38 and the therapeutic product is MER Proto-Oncogene, Tyrosine Kinase (MERTK); (37) retinitis pigmentosa 40 and the therapeutic product is Phosphodiesterase 6B (PDE6B); (38) retinitis pigmentosa 41 and the therapeutic product is Prominin 1 (PROM1); (39) retinitis pigmentosa 56 and the therapeutic product is Interphotoreceptor Matrix Proteoglycan 2 (IMPG2); (40) petinitis pigmentosa 62 and the therapeutic product is Male Germ Cell Associated Kinase (MAK); (41) retinitis pigmentosa 80 and the therapeutic product is Intraflagellar Transport 140 (IFT140); or (42) Best disease and the therapeutic product is Bestrophin 1 (BEST1).

### 4.6 Assays

The skilled artesian may use the assays as described herein and/or techniques known in the art to study the composition and methods described herein, for example to test the formulations provided herein. As detailed in Section 5, the following assays are also provided herein.

### 4.6.1 Ultrasound B-Scan

A high-frequency ultrasound (U/S) probe (UBM Plus; Accutome, Malvern, PA, USA) can be used to determine SCS thickness by generating 21) cross-sectional images of the SCS in animal eyes ex vivo after injecting different volumes ranging in viscosity and/or elastic modulus (G′) (e.g., from 25 μL to 500 μL ranging from low viscosity to high viscosity) at about 32-35° C. An U/S probe cover (Clearscan, Eye-Surgical-Instruments, Plymouth, MN) can be attached to the UBM Plus to facilitate U/S image acquisition. The U/S probe can be used to acquire sagittal views around the eye (e.g., eight sagittal views). Postprocessing of the U/S B-scans can be performed to find the thickness from the outer sclera to the inner retina at, for example, 1, 5, and 9 mm posterior to the scleral spur. The mean, median, and standard deviation for each eye can be calculated.

### 4.6.2 Measuring SCS Thickness Based on Liquid Volume

3D cryo-reconstruction imaging can be used to measure SCS thickness. Animal eyes that are injected with, for example, 25 μL to 500 μL containing red-fluorescent particles are frozen a few minutes (e.g., 3-5 minutes) post injection and prepared for cryosectioning. Using a digital camera, one red-fluorescent image of the cryoblock of tissue can be obtained every 300 μm by slicing the sample with the cryostat. Image stacks consisting of red-fluorescence images are analyzed to determine SCS thickness.

### 4.6.3 Measuring SCS Thickness Based on Formulation

U/S B-scan can be used to determine SCS thickness after injection of pharmaceutical compositions ranging in viscosity and/or elastic modulus (G′) into the SCS of animals. High-frequency ultrasound B-scan can be used to determine the rate of SCS collapse. Eight sagittal views over the pars plana can be acquired: (a) supranasal, over the injection site; (b) superior; (c) nasal; (d) supratemporal; (e) temporal; (f) infratemporal; (g) inferior; and (h) infranasal.

Off-line post processing can be performed on the U/S views to measure the SCS thickness. The U/S probe can have a minimum axial resolution of 15 m. For each U/S view, a line segment 5 mm posterior to the scleral spur and perpendicular to the sclera can be created. A line can start at the outer surface of the sclera and end at the inner surface of the retina. The sclera and chorioretina can be included in the measurement to ensure the line is perpendicular. SCS thickness is then calculated by subtracting the tissue thickness from the measured line length. Curve fitting is done to determine the rate of SCS collapse.

U/S B-scan can be used to determine SCS thickness at multiple locations over time and the rate of SCS collapse can be calculated. The approximate clearance rate of injected fluorescent material from the SCS can be found by taking fluorescence fundus images in the animal eyes in vivo over time until fluorescence is no longer detected.

### 4.6.4 SCS Clearance Kinetics by Fundus Imaging

To study the effect of viscosity and/or elastic modulus (G′) on movement in the SCS, different pharmaceutical compositions ranging in viscosity and/or elastic modulus (G′) and containing a fluorescein can be injected into the SCS. The approximate clearance rate or clearance time of injected fluorescent material from the SCS can be found by taking fluorescence fundus images over time in animal eyes in vivo. In some cases, the rate of clearance can be determined by determining the total clearance time and the clearance time constant (tclearance) calculated using a curve fit derived from the normalized concentration of total fluorescent signal over time. Topical eye drops of tropicamide and phenylephrine (Akorn, Lake Forest, IL) can be administered prior to each imaging session to dilate the eye. A RetCam II (Clarity Medical Systems, Pleasanton, CA) with the 1300 lens attachment and the built-in fluorescein angiography module can be used to acquire the images. Multiple images can be taken with the blue light output from the RetCam II set at, for example, 0.0009, 1.6, and 2.4 W/m2. In an attempt to capture the entire interior surface of the ocular globe, nine images can be captured: central, supranasal, superior, supratemporal, temporal, infratemporal, inferior, infranasal, and nasal. This allows imaging into the far periphery. Imaging can be done immediately after injection, at 1 h, every 3 h for 12 h, and every two days post-injection. The total clearance time, which can be defined as the first time point in which fluorescence is not detectable by visual observation, is determined for all eyes injected. Fluorescein isothiocyanate-conjugated AAV (FITC-AAV), or FITC Conjugated-AAV capsid Protein-specific monoclonal antibody may be utilized in analogous experiments to track movement and clearance of AAV particles in the SCS. Methods for fluorescent labeling of AAV are known in the art (Shi, et al. Sci. Adv. 2020; 6: eaaz3621; and Tsui, T. Y., et al. Hepatology 42, 335-342 (2005). Antibodies (FITC Conjugated) recognizing many AAV serotypes are commercially available.

### 4.6.5 Flat Mount to Characterize 2D Circumferential Spread

Pharmaceutical compositions of the present disclosure containing fluorescein, or fluorescently labeled AAV, are injected into the SCS. After SCS injection and freezing, eyes can be prepared to assess the 2D spread of particles and fluorescein. The frozen eye are sliced open from the limbus to the posterior pole to generate equidistant scleral flaps. The resulting scleral flaps are splayed open and the frozen vitreous humor, lens, and aqueous humor are removed.

A digital SLR camera (Canon 60D, Canon, Melville, N.Y.) with a 100 mm lens (Canon) can be used to acquire brightfield and fluorescence images. Camera parameters are held constant. To acquire the area of fluorescein spread, a green optical band-pass filter (520±10 nm; Edmunds Optics, Barrington, N.J.) can be placed on the lens, and the sample can be illuminated by a lamp with the violet setting of a multicolor LED bulb (S Series RGB MR16/E26. HitLights, Baton Rouge, La.). To visualize the location of the red-fluorescent particles, a red filter (610±10 nm; Edmunds Optics) can be placed on the lens, and the sample can be illuminated with the same lamp switched to green light. The area of green and red fluorescence that are above threshold can be calculated for each eye using ImageJ (National Institutes of Health, Bethesda, Md.). Thresholding can be set manually based on visual inspection of background signal.

### 4.6.6 Intraocular Pressure Measurements

A pressure measurement system can be used to measure pressure in SCS after SCS injection. Animals can be terminally anesthetized by subcutaneous injection of a ketamine/xylazine cocktail. After SCS injection (N=4), pressure in the SCS can be measured every few minutes. Pressures are monitored until they reach their original baseline values from before injection (i.e., ˜15 mmHg). After the measurements, the animals are euthanized with a lethal dose of pentobarbital injected intravenously. A second set of SCS injections can be made in the animal postmortem. In postmortem measurements, pressure is only measured in the tissue space (i.e., SCS) where the injection was made.

### 4.6.7 Temperature Stress Assay

A temperature stress development stability study can be conducted at 1.0×10¹² GC/mL over 4 days at 37° C. to evaluate the relative stability of formulations provided herein. Assays can be used to assess stability include but are not limited to in vitro relative potency (IVRP), vector genome concentration (VGC by ddPCR), free DNA by dye fluorescence, dynamic light scattering, appearance, and pH. Long-term development stability studies can be carried out for 12 months to demonstrate maintenance of in-vitro relative potency and other quality at −80° C. (≤−60° C.) and −20° C. (−25° C. to −15° C.) in the formulations provided herein.

### 4.6.8 In Vitro Relative Potency (IVRP) Assay

To relate the ddPCR GC titer to gene expression, an in vitro bioassay may be performed by transducing HEK293 cells and assaying the cell culture supernatant for anti-VEGF Fab protein levels. HEK293 cells are plated onto three poly-D-lysine-coated 96-well tissue culture plates overnight. The cells are then pre-infected with wild-type human Ad5 virus followed by transduction with three independently prepared serial dilutions of AAV vector reference standard and test article, with each preparation plated onto separate plates at different positions. On the third day following transduction, the cell culture media is collected from the plates and measured for VEGF-binding Fab protein levels via ELISA. For the ELISA, 96-well ELISA plates coated with VEGF are blocked and then incubated with the collected cell culture media to capture anti-VEGF Fab produced by HEK293 cells. Fab-specific anti-human IgG antibody is used to detect the VEGF-captured Fab protein. After washing, horseradish peroxidase (HRP) substrate solution is added, allowed to develop, stopped with stop buffer, and the plates are read in a plate reader. The absorbance or OD of the HRP product is plotted versus log dilution, and the relative potency of each test article is calculated relative to the reference standard on the same plate fitted with a four-parameter logistic regression model after passing the parallelism similarity test, using the formula: EC50 reference÷EC50 test article. The potency of the test article is reported as a percentage of the reference standard potency, calculated from the weighted average of the three plates.

To relate the ddPCR GC titer to functional gene expression, an in vitro bioassay may be performed by transducing HEK293 cells and assaying for transgene (e.g. enzyme) activity. HEK293 cells are plated onto three 96-well tissue culture plates overnight. The cells are then pre-infected with wild-type human adenovirus serotype 5 virus followed by transduction with three independently prepared serial dilutions of enzyme reference standard and test article, with each preparation plated onto separate plates at different positions. On the second day following transduction, the cells are lysed, treated with low pH to activate the enzyme, and assayed for enzyme activity using a peptide substrate that yields increased fluorescence signal upon cleavage by transgene (enzyme). The fluorescence or RFU is plotted versus log dilution, and the relative potency of each test article is calculated relative to the reference standard on the same plate fitted with a four-parameter logistic regression model after passing the parallelism similarity test, using the formula: EC50 reference÷EC50 test article. The potency of the test article is reported as a percentage of the reference standard potency, calculated from the weighted average of the three plates.

### 4.6.9 Vector Genome Concentration Assay

Fluorescein isothiocyanate-conjugated AAV (FITC-AAV), or FITC Conjugated-AAV capsid Protein-specific monoclonal antibody may be utilized in analogous experiments to track movement and clearance of AAV particles in the SCS. Methods for fluorescent labeling of AAV are known in the art (Shi, et al. Sci. Adv. 2020; 6: eaaz3621; and Tsui, T. Y., et al. Hepatology 42, 335-342 (2005). Antibodies (FITC Conjugated) recognizing many AAV serotypes are commercially available.

### 4.6.10 Free DNA Analysis Using Dye Fluorescence Assay

Free DNA can be determined by fluorescence of SYBR® Gold nucleic acid gel stain (‘SYBR Gold dye’) that is bound to DNA. The fluorescence can be measured using a microplate reader and quantitated with a DNA standard. The results in ng/μL can be reported.

Two approaches can be used to estimate the total DNA in order to convert the measured free DNA in ng/μL to a percentage of free DNA. In the first approach the GC/mL (OD) determined by UV-visible spectroscopy was used to estimate the total DNA in the sample, where M is the molecular weight of the DNA and 1×10⁶ is a unit conversion factor:

Total DNA (ng/μL) estimated=1×10⁶×GC/mL(OD)×M (g/mol)/6.02×10²³

In the second approach, the sample can be heated to 85° C. for 20 min with 0.05% poloxamer 188 and the actual DNA measured in the heated sample by the SYBR Gold dye assay can be used as the total. This therefore has the assumption that all the DNA was recovered and quantitated. For trending, either the raw ng/μL can be used or the percentage determined by a consistent method can be used.

### 4.6.11 Size Exclusion Chromatography (SEC)

SEC can be performed using a Sepax SRT SEC-1000 Peek column (PN 215950P-4630, SN: 8A11982, LN: BT090, 5 μm 1000A, 4.6×300 mm) on Waters Acquity Arc Equipment ID 0447 (C3PO), with a 25 mm pathlength flowcell. The mobile phase can be, for example, 20 mM sodium phosphate, 300 mM NaCl, 0.005% poloxamer 188, pH 6.5, with a flow rate of 0.35 mL/minute for 20 minutes, with the column at ambient temperature. Data collection can be performed with 2 point/second sampling rate and 1.2 nm resolution with 25 point mean smoothing at 214, 260, and 280 nm. The ideal target load can be 1.5×10¹¹ GC. The samples can be injected with 50 μL, about ⅓ of the ideal target or injected with 5 μL.

### 4.6.12 Dynamic Light Scattering (DLS) Assay

Dynamic light scattering (DLS) can be performed on a Wyatt DynaProIII using Corning 3540 384 well plates with a 30 μL sample volume. Ten acquisitions each for 10 s can be collected per replicate and there can be three replicate measurements per sample. The solvent can be set according to the solvent used in the samples, for example ‘PBS’ for an AAV vector in dPBS. Results not meeting data quality criteria (baseline, SOS, noise, fit) can be ‘marked’ and excluded from the analysis.

### 4.6.13 Viscosity Measurement

Viscosity can be measured using methods known in the art, for example methods provide in the United States Pharmacopeia (USP) published in 2019 and previous versions thereof (incorporated by reference herein in their entirety). Viscosity at low shear was measured using a capillary viscometer, using methods described in USP<911>.

Viscosity versus shear rate can be determined using a cone and plate rotational rheometer. Rheometry measurements are described in the United States Pharmacopeia (USP) USP<1911> and rotational viscometry is described in USP<912>. Rotational rheometry viscosity measurements can be collected with an AR-G2 rheometer equipped with a Peltier temperature control plate with a 60 mm 1° angle aluminum cone accessory (TA Instruments, New Castle, DE). A viscosity versus shear rate sweep can be performed over the range starting at <0.3 s−1 ramped up to 5000 s⁻¹ with 5 points per decade collected. The viscosity versus shear rate was collected at 20° C. Viscosity at 10,000 and 20,000 s⁻¹ were extrapolated from the data.

In some cases, the viscosity of the pharmaceutical composition or the reference pharmaceutical composition can be measured at zero, 0.1 s⁻¹, 1 s⁻¹, 1000 s⁻¹, 5000 s⁻¹, 10,000 s⁻¹, 20,000 s⁻¹, or more than 20,000 s⁻¹.

### 4.6.14 Rheology Measurements

Rheometry measurements are described in the United States Pharmacopeia (USP) USP<1911> and rotational viscometry is described in USP<912>.

Rheology measurements were collected with an AR-G2 rheometer equipped with a Peltier temperature control plate with a 60 mm 1° angle aluminum cone accessory (TA Instruments, New Castle, DE)

The gelation temperature was determined using by applying at temperature ramp in oscillatory mode at 0.1% strain and 1 Hz. The samples were loaded and pre-equilibrated for 5 minutes at 5° C., followed by a temperature ramp at 5° C./min up to either 40° C. or 60° C. The temperature at which the storage/elastic modulus (G′) and loss/viscous modulus (G″) crossed was recorded as the system gelation temperature. A torque sweep demonstrated that linear viscoelastic region extended to about 0.4% and therefore operation at 0.1% was well-within the linear viscoelastic region.

Gelation time was determined in oscillatory mode 0.1% strain and 1 Hz. Samples equilibrated at both 5° C. and 20° C. and exposed to a temperature jump to 34° C. As above, the time to gel was defined as the crossover of the storage and loss modulus curves.

### 4.6.15 Virus Infectivity Assay

TCID₅₀ infectious titer assay as described in François, et al. Molecular Therapy Methods & Clinical Development (2018) Vol. 10, pp. 223-236 (incorporated by reference herein in its entirety) can be used. Relative infectivity assay as described in Provisional Application 62/745,859 filed Oct. 15, 2018) can be used.

### 4.6.16 Differential Scanning Fluorimetry

The thermal stability of proteins and virus capsids made up of proteins can be determined by differential scanning fluorimetry (DSF). DSF measures the intrinsic tryptophan and tyrosine emission of proteins as a function of temperature. The local environment of Trp and Tyr residues changes as the protein unfolds resulting in a large increase in fluorescence. The temperature where 50% of proteins are unfolded is defined as the ‘melting’ temperature (Tₘ). Fluorescence spectroscopy is described in the USP<853> and USP<1853>.

DSF data was collected using a Promethius NTPlex Nano DSF Instrument (NanoTemper technologies, Munich, Germany). Samples were loaded into the capillary cell at 20° C. and the temperature ramped at a rate of 1° C./min to 95° C. The signal output ratio of emission at 350 nm (unfolded) and 330 nm (unfolded) was used to determine the Tₘ.

### 4.6.17 Injection Pressure Measurements

Injection pressures were measured using either a Flow Screen and Fluid Sensor (Viscotec America, Kennesaw, GA) or a PressureMAT-DPG with single use pressure sensor S-N-000 (PendoTECH, Princeton, NJ).

Injections into air were either performed manually or using a Legato-100 syringe pump (Kd Scientific, Holliston, MA) to apply a consistent flow rate. For injections into enucleated porcine eyes, the eyes were mounted on a Mandell eye mount (Mastel) with applied suction to adjust the intraocular pressure of the eye.

### 4.6.18 Reference Compositions

The viscosity of a composition provided herein may be evaluated by comparing the composition to a reference pharmaceutical composition. In some embodiments, the reference pharmaceutical composition is a pharmaceutical composition comprising the same type and amount of recombinant AAV as the composition being evaluated, but is not a thermoresponsive composition. In some embodiments, the reference pharmaceutical composition is a pharmaceutical composition comprising the same type and amount of recombinant AAV as the composition being evaluated, but has a lower viscosity and/or elastic modulus at extraocular temperature (about 32-35° C.) than the composition being evaluated.

In some embodiments, the reference pharmaceutical composition is a pharmaceutical composition comprising the same recombinant AAV in the same concentration as the composition being evaluated in phosphate-buffered saline. In some embodiments, the reference pharmaceutical composition is a pharmaceutical composition comprising the same recombinant AAV in the same concentration as the composition being evaluated in Dulbecco's phosphate buffered saline with 0.001% poloxamer 188, pH 7.4. In some embodiments, the reference pharmaceutical composition is a pharmaceutical composition comprising the same recombinant AAV in the same concentration as the composition being evaluated in Dulbecco's phosphate buffered saline with 4% sucrose and 0.001% poloxamer 188, pH 7.4. A reference pharmaceutical composition may be administered by the same route or a different route as the composition being evaluated. In some embodiments, the reference pharmaceutical composition is administered suprachoroidally.

### 5. EXAMPLES

The examples in this section (i.e., section 5) are offered by way of illustration, and not by way of limitation.

### 5.1 Example 1: Optimization of Thermoresponsive Gel Formulation for Suprachoroidal Delivery

### 5.1.1 Overview of Thermoresponsive Gel Formulation Objectives

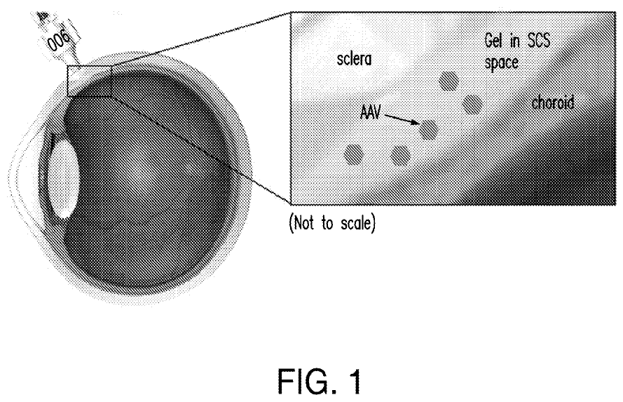

Construct II is being investigated as a treatment delivered by injection into the suprachoroidal space. The suprachoroidal space (SCS) is a region between the sclera and the choroid that expands upon injection of the drug solution (Habot-Wilner, 2019). See also FIG. 1. The SCS space recovers to its pre-injection size as the injected solution is cleared by physiologic processes. The drug solution diffuses within SCS and is absorbed into adjacent tissues. Capillaries in the choroid are permeable to low molecular weight osmolytes. This example describes experimental approaches to increase residence time of Construct II in the suprachoroidal space and to ultimately improve its efficacy.

In order to achieve a longer residence time, adeno-associated virus (AAV) was formulated in a final formulation that is an injectable liquid when at a temperature between refrigerated conditions (2-8° C.) and controlled room temperature (20° C.) and then changes state to a gel at the temperature of the eye (34.5±0.8° C.). The gel holds the AAV in the suprachoroidal space, reducing clearance and increasing localization, thereby resulting in enhanced therapeutic efficacy in the desired target tissues.

### 5.1.2 Overview of Initial Design Parameters

Initial design parameters for formulation feasibility are subject to adjustment with additional data and described here to demonstrate the starting point for formulation optimization.

Injection to the eye is a sensitive injection procedure as the eye is a critical organ. The needle gauge cannot be too high when injecting a drug in the eye in order to avoid pain, tissue damage, or inflammation. For example, in some cases, a 30 gauge can be selected and in other cases, a 29 gauge is selected. For injection into the eye, it must be possible to inject the solution through a very narrow bore needle within a specified time frame before it gels and plugs the needle and injection site. This adds limits to the viscosity of the formulation at room temperature (or refrigerated if injected cold) and also to the gelation time to allow the injection to be complete. This example describes a response surface design of experiments approach to optimize a formulation composition that can simultaneously meet all the requirements identified for suprachoroidal delivery.

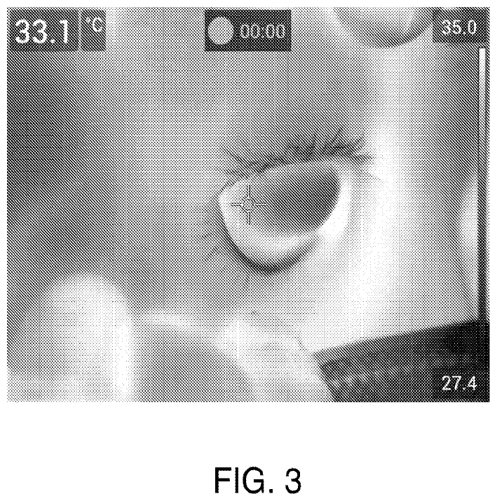

The formulation should gel at the temperature of the eye. The temperature of the surface of the eye has been reported as 34.5±0.8° C. (Tkacova et al., 2011, MEASUREMENT 2011, Proceedings of the 8th International Conference, Smolenice, Slovakia). FIG. 3. Shows a measured extraocular temperature at 33.1° C. using a thermal camera (FLIR, model T530). The temperature of the surface of the eye can be taken as a worst case for the suprachoroidal space temperature, which may be slightly warmer than the surface. The limit for gelation temperature is the temperature of the eye (34.5° C.). The average minus three standard deviations of the eye temperature is 32° C. Therefore, a preferred gelation temperature of ≤32° C. was used a design parameter to ensure that gelation will occur in the eye.

The thermoresponsive gel should be an injectable liquid when at a temperature between refrigerated conditions (2-8° C.) and controlled room temperature (20° C.). Ideally, for ease of dose preparation and manufacturing, the gel temperature should be sufficiently above room temperature to allow for variability in room temperature and for practical mixing, pumping and other operations to be performed. For example, a gelation temperature of ≥27° C. might be more easily handled. The higher gelation temperature (≥27° C.) is preferred but is not a rigid requirement because the dose could be chilled to a lower temperature for dose preparation and if needed also chilled before administration.

The formulation should have an acceptable viscosity that will allow for an injection using commonly available syringe components (i.e. an injection pressure limit based on syringe pressure-rating limitations). There are conflicting design parameters to balance between gel-forming temperature and time and injectability.

The desired needle size is 30 or 29 gauge, and this will impact the pressure of the injection. Gelation should not occur before the injection is complete to avoid clogging the needle or injection site. For initial modelling, an injection time of 10 seconds was assumed.

There are alternative approaches to reducing the injection pressure, if needed. The temperature, viscosity, gelation time, and the required injection time are linked parameters. The Hagen-Poiseuille equation (ΔP=(8 μLQ)/(πR⁴), see below) shows that the injection pressure and time (dictated by the flow rate) are linearly linked. Therefore, doubling the injection time from 10 s to 20 s will reduce the pressure by a factor of two. The viscosity and pressure are inversely correlated. Reducing the viscosity, such as by a reduction in temperature, will reduce the injection pressure. The time to gel is greater if the dose is chilled. Chilling of the dose will lower viscosity and the injection pressure. A chilled dose will also take longer to gel, allowing for a slower injection which can also reduce the injection pressure.

The gelation time should be greater than the injection time to avoid clogging the needle or injection site and should be less than the expected clearance time of 10 minutes (600 s) for pressure driven reflux reported in the literature (Chiang, IOVS, 2017, 58 (1) 545-554). An initial design preferred target of <90 seconds is considered (if feasible) for the formulation evaluation as a very conservative gelation time compared to the 600 s reflux clearance time expected. Medium and longer-term clearance of AAV from the SCS spaces by blood flow, erosion of the gel, diffusion and convention is expected to be significantly reduced after gelation has occurred.

If possible, within the design space identified it is further desired to have some room for the gel to be slightly diluted upon injection while still maintain the ability to gel at the same or similar temperature.

TABLE 2
INITIAL DESIGN PARAMETERS

| Parameter | Target | Rationale |
|---|---|---|
| Excipients safety | Poloxamer 407 and 188 | Safety |
| | excipients are safe and | |
| | have been used | |
| | previously in | |
| | thermoresponsive gel | |
| | formulations | |
| Stability of Vector | Vector is stable and | Stability is required to ensure efficacy |
| | retains potency | |
| Gelation | Preferred: 27° C. < T | Upper limit is the temperature of the eye surface (34° C.) and |
| Temperature | (gel) < 32° C. | the range is this minus 3 standard deviations (32° C.) |
| | Range: 20° C. < T | Lower limit is based on practicality of working with as low |
| | (gel) < 32° C. | viscosity as possible at room temperature for dose |
| | Limit: 20° C. < T | administration and manufacturing, a range of 27° C. to 32° C. |
| | (gel) < 34.5° C. | was used for initial assessment. |
| | | Formulation may be chilled for certain operations and even |
| | | for dosing if required based on viscosity |
| Gelation Time | Preferred: 90 s > time | Greater than the injection time. |
| | (gel) > 15 s | Gelation time should be short enough to avoid clearance or |
| | Range: 600 s > time | dissipation of the dose before fully gelled. |
| | (gel) > 10 s | Gel time can be increased by chilling of the dose if needed. |
| Injection Time | Preferred: 10-15 s | Clinical administration targets |
| | Range: 5-30 s | |
| Administration | Preferred: 29 or 30 Ga | Clinical administration requirement and ability to use standard |
| components | ETW needle (ID = 220 | components |
| | or ID = 240 μm) and | |
| | standard plastic syringe | |
| Injection Pressure | Preferred ≤43 PSI | Preferred is based on ISO 7886-1: 2017 for syringe pressure |
| | Range ≤65 PSI | ratings, and low/easy injection pressure and force. |
| | Limit ≤100 PSI | The range of 65 PSI is based on the human factor/feeling of |
| | | force needed during laboratory injection feasibility |
| | | experiments |
| | | The limit of 100 PSI is based on level lower than 125 PSI |
| | | typical pressure rating of plastic syringes |
| Viscosity | Preferred ≤183 mPas at | Based on injection pressure target of 65 PSI when using |
| | room temp | CLSD 30 Ga ETW (220 μm ID) needle (see Table 3) at room |
| | Initial target ≤183 mPas | temperature or when chilled. |
| | when chilled | This target could be widened with use of larger needles or |
| | | allowing for higher pressure syringe, so is an initial target |
| | | rather than an absolute requirement |

### 5.1.3 Viscosity Design Space

The Hagen-Poiseuille equation for pressure drop during flow through a needle is given by ΔP=(8 μLQ)/(πR⁴). The pressure depends upon the viscosity (μ), the needle length (L), the volumetric flow rate (Q), and the inner radius of the needle (R). The equation was used to calculate the pressure drop in pounds per square inch (PSI) as a function of viscosity for 30 gauge and 29 gauge needles (ISO 9626:2016: regular wall, RW; thin wall, TW; extra thin wall, ETW; and ultra thin wall, UTW, and additional ClearSide (CLSD) needles in design or used in development studies). A conversion factor of PSI=Pa/6894.76 was used to convert to PSI. The total needle length including the mounting length is 14 mm, the injection volume is 0.1 mL, the injection time is modelled at 10 s (Q=0.1 mL/10=0.01 mL/s), and the needle inner diameters considered are: 30 Ga/29 Ga (133 μm ID), 30 Ga TW (165 μm ID), 30 Ga ETW/29 Ga TW (190 μm ID), 30 Ga UTW/29 Ga ETW (240 μm ID), ClearSide (CLSD) brand 30 Ga needle (160 μm ID), CLSD 30 Ga ETW (220 μm ID), CLSD 29 Ga ETW (240 μm ID).

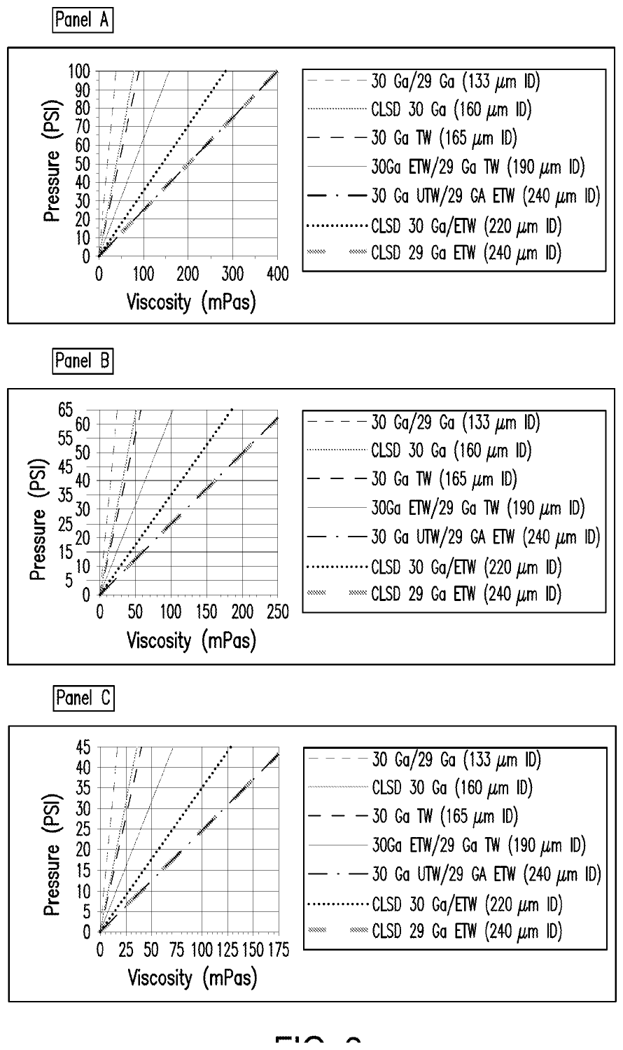

The pressure versus viscosity calculation results are shown in FIG. 2. and the results tabulated for the preferred, target, and limit values in Table 3. Based on the calculations in Table 3, the initial design assessment was based on the target pressure of 65 PSI using the CLSD 30 Ga ETW (220 μm ID) needle, which corresponds to a viscosity of 183 mPas.

The relative decrease in pressure and/or time at constant pressure needed to inject the CLSD 30 Ga ETW (220 μm ID) or CLSD 29 Ga ETW (240 μm ID) compared to the CLSD 30 Ga needle (160 μm ID) is based on the ratio of the needle inner diameter to the fourth power and is: (220/160)⁴=3.6-fold and (240/160)⁴=5-fold respectively. Therefore, initial feasibility data with the current 160 μm ID 30 Ga CLSD needle can be used to extrapolate the expected pressure and time for the use of the 30 and 29 Ga ETW needles planned for the clinical delivery of the gel formulations.

TABLE 3
VISCOSITY DESIGN SPACE BASED
ON INJECTION PRESSURES

| | Viscosity (mPas) upper range | | |
|---|---|---|---|
| | for ≤43 | for 65 | for 100 |
| | PSI | PSI | PSI |
| Needle | (Preferred) | (Target) | (Limit) |
| 30 Ga/29 Ga (133 μm ID) | 16 | 24 | 38 |
| 30 Ga TW (165 μm ID) | 38 | 58 | 90 |
| 30 Ga ETW/29 Ga TW (190 μm ID) | 67 | 102 | 157 |
| 30 Ga UTW/29 Ga ETW (240 μm ID) | 171 | 259 | 400 |
| CLSD 30 Ga needle (160 μm ID) | 34 | 51 | 79 |
| CLSD 30 Ga ETW (220 μm ID) | 121 | 183* | 282 |
| CLSD 29 Ga ETW (240 μm ID) | 171 | 259 | 400 |

*Initial formulation design feasibility assessment set for 30 Ga TW (220 μm ID) needle and expected ~65 PSI nominal injection pressure

### 5.1.4 Identification of Design Space for Thermoresponsive Hydrogel Formulation Composition

To maintain consistency with the current modified DPBS with sucrose formulation and also to maintain tonicity of the fluid within the hydrogel matrix within physiologically acceptable ranges (240<osmolality<600 mOsm/kg) the hydrogel will be based on dissolution of poloxamer 407 and poloxamer 188 into the current modified DPBS with sucrose formulation buffer (osmolality=345 mOsm/kg).

A combination of poloxamer 407 and poloxamer 188 was evaluated in a design of experiments approach to identify if there is a formulation composition with a gel temperature in desired target range while also limiting the viscosity to a level that can be injected through a 30 or 29 gauge needle.

The impact of the composition of the hydrogel formulation on the gelation temperature, and the gelation time and viscosity at both 5° C. and 20° C. was assessed using a response surface central composite design of experiments (DOE) approach utilizing JMP 15 software (Cary, NC). A DOE approach was needed based on the number of formulation design limitations required that have differing responses to composition changes and to enable to determination of one or more optimal target formulations. Poloxamer 407 was varied from 16% to 22% w/v and poloxamer 188 from 0% to 16% w/v with a duplicate center point. Gelation can be determined using different methods. Some method may measure the onset of gelation and others may measure an objective ‘crossover’ in material properties as the gelation temperature. For the purposes of the formulation design the crossover in G′ and G″ as a function of temperature was taken as the gelation temperature for consistency and objectivity. The raw data shows that the onset of gelation occurs at a slightly lower temperature than the crossover.

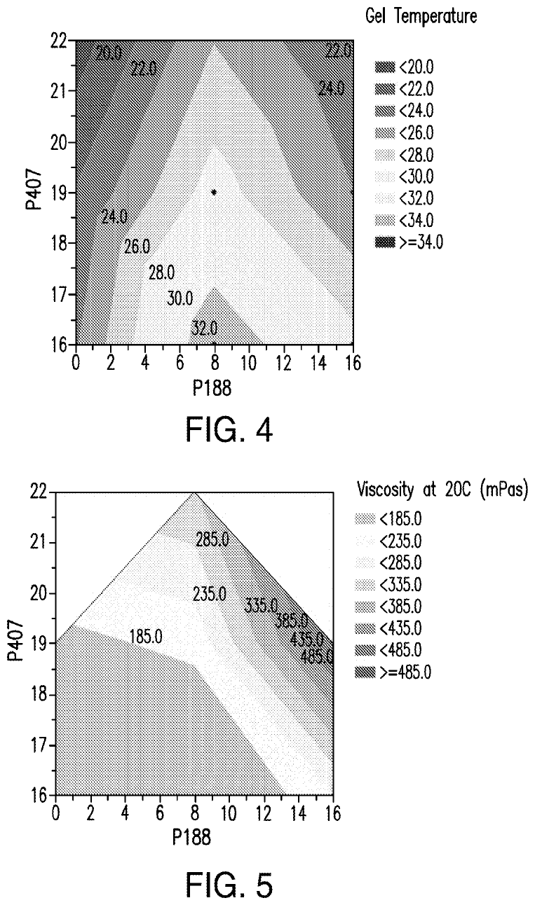

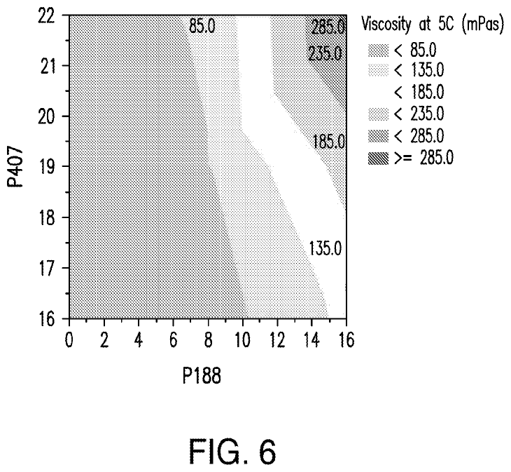

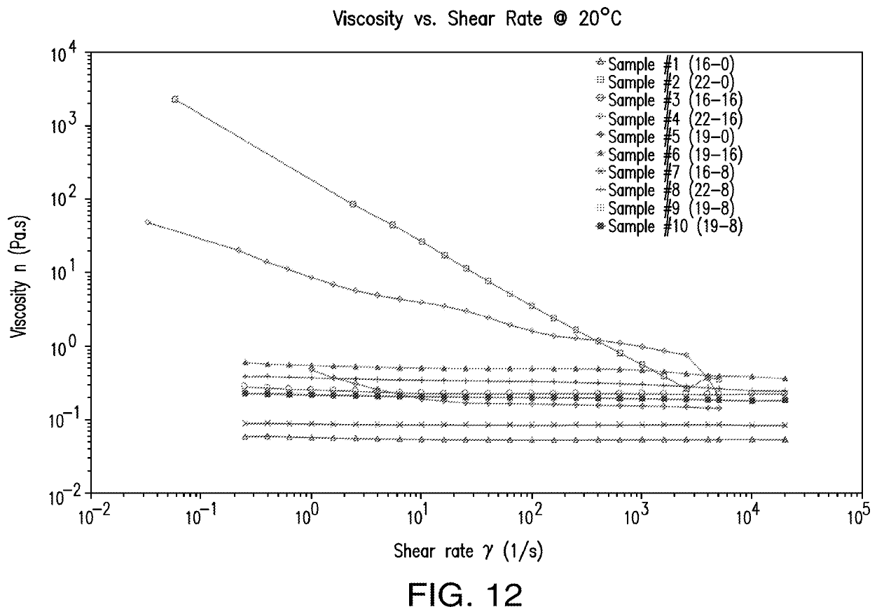

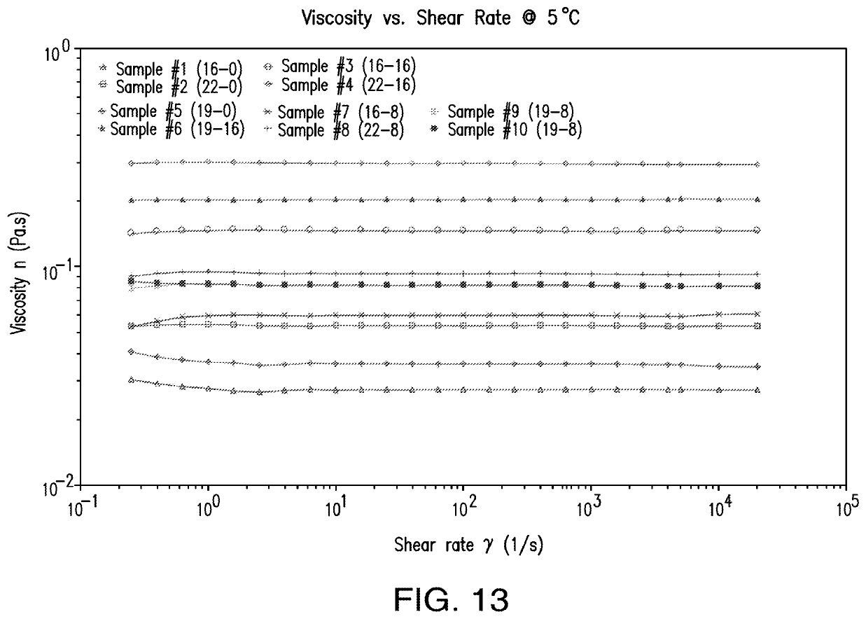

The results of the DOE study are summarized in Table 4. The data was fit in JMP DOE software to produce a response surface for the different measurements, FIG. 4. shows the gelation temperature as a function of formulation composition response surface, Responses surfaces for viscosity as a function of composition are shown in FIG. 5 (20° C.) and FIG. 6. (5° C.) and a summary of the raw shear rate sweep data is shown in FIG. 12. and FIG. 13.

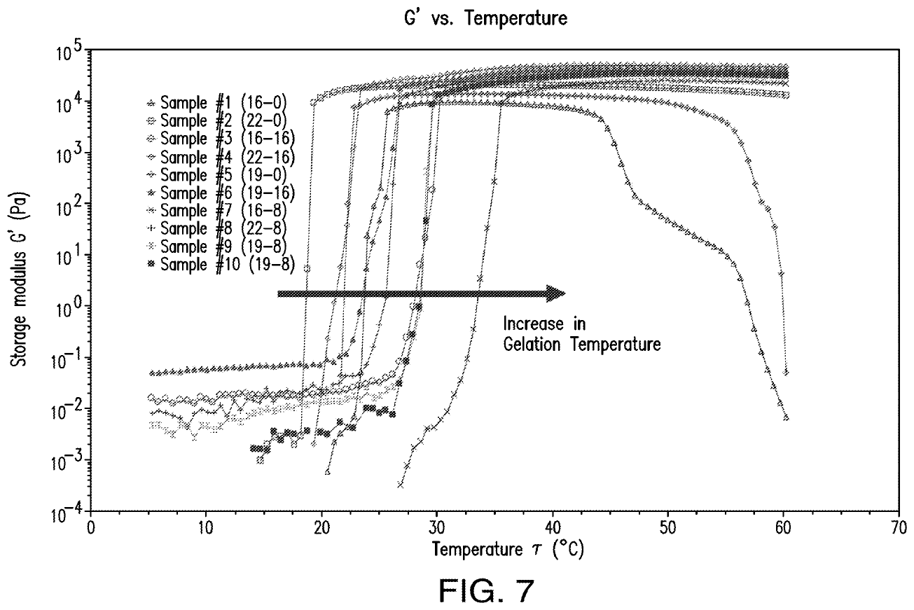

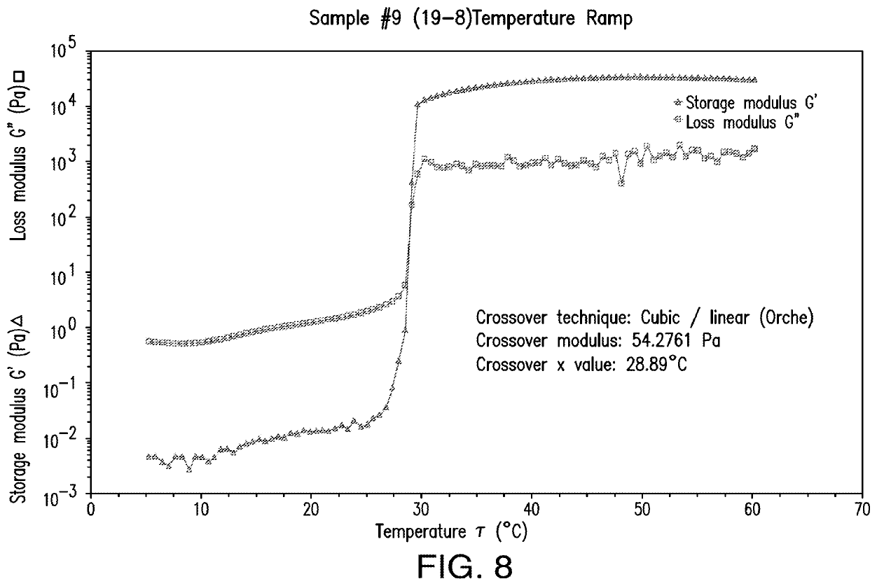

The raw data for gelation profiles are summarized in FIG. 7, and a specific example for how the gelation temperature is determined as the crossover in G′ and G″ for sample #9 shown in FIG. 8. The raw data shows that the onset of gelation occurs at a slightly lower temperature than the crossover. Therefore, the use as the crossover as the definition of gelation temperature for the formulation adds extra robustness to the design because complete gelation is not necessarily needed to dramatically increase localization of the AAV in the SCS space.

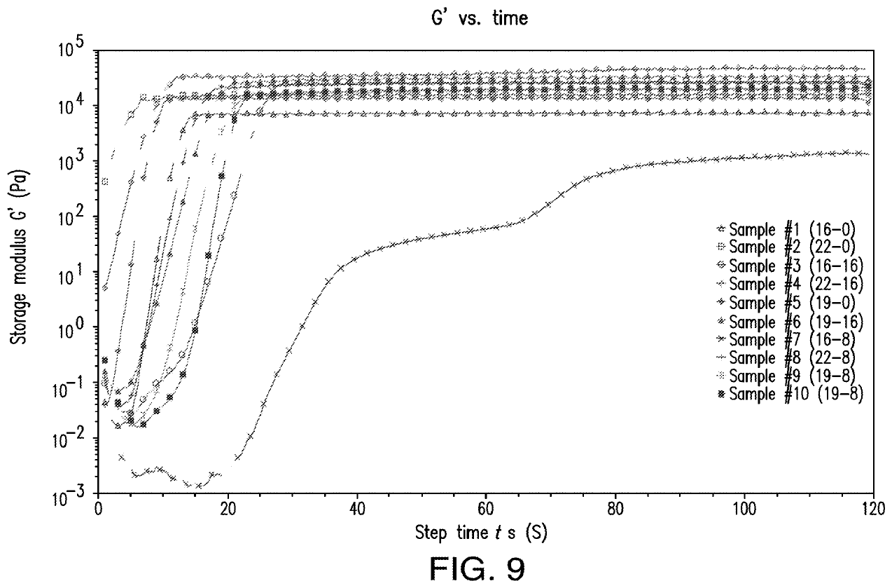

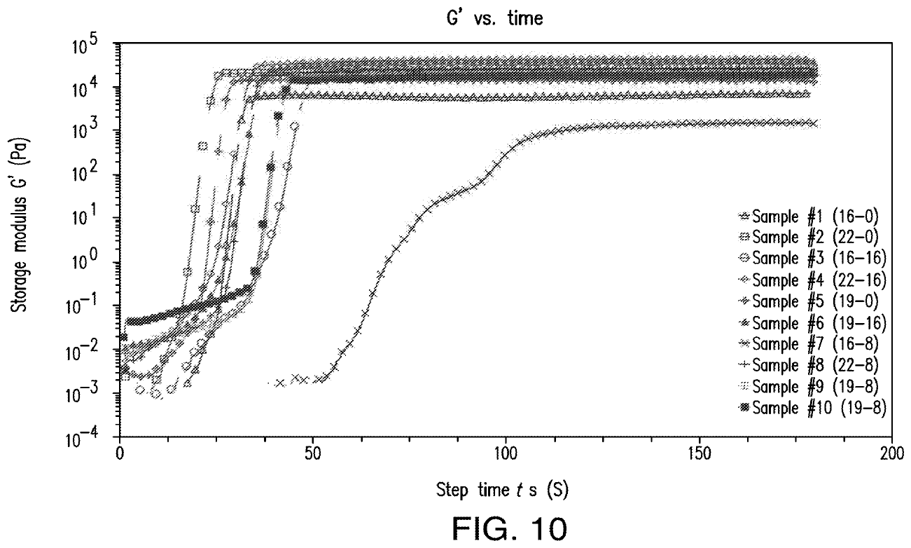

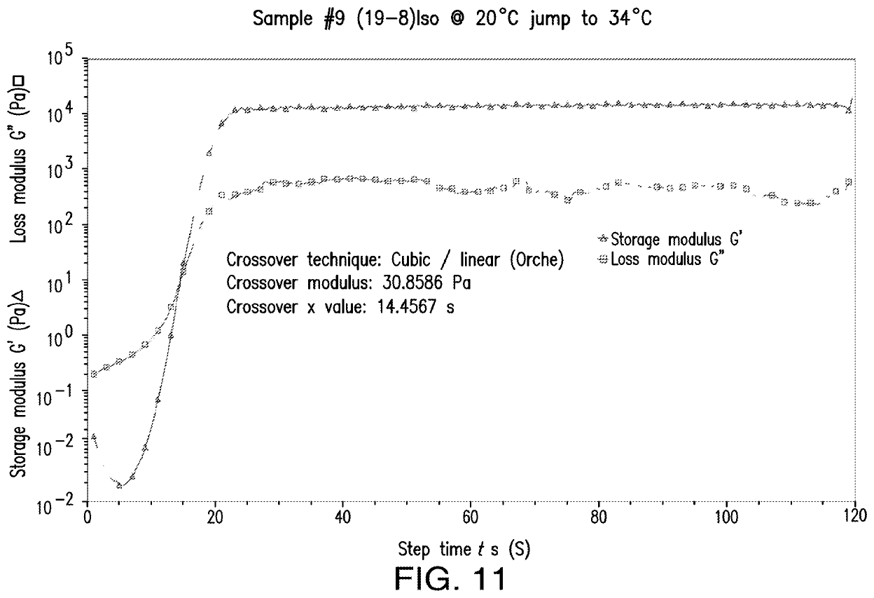

FIG. 9. and FIG. 10. show the G′ versus time used to determine the gelation times for the samples exposed to a temperature jump from 5° C. and 20° C. to 34° C. respectively. FIG. 11. shows the specific example for sample #9.

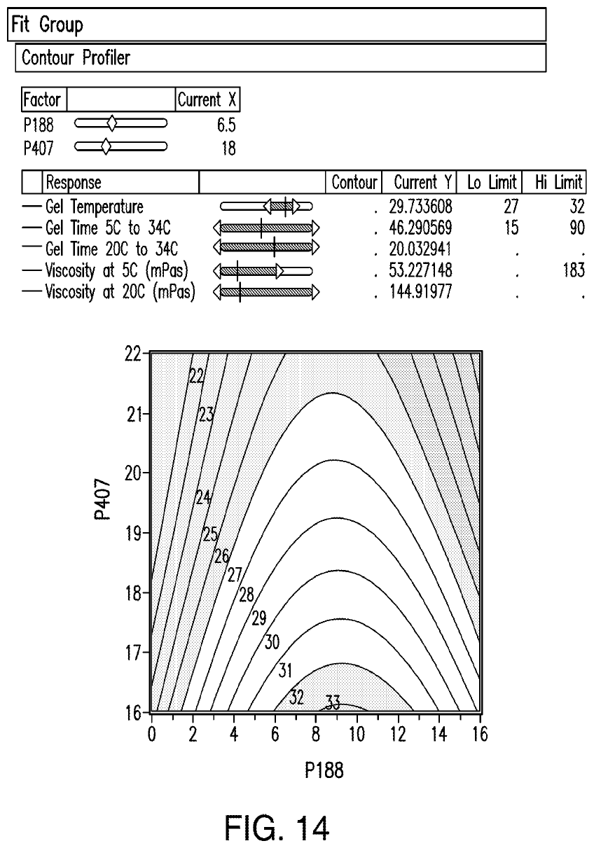

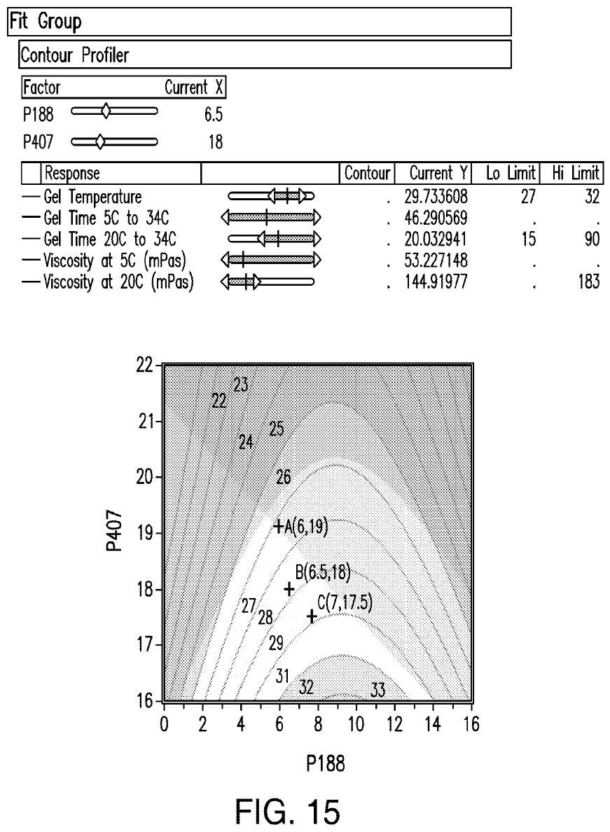

The data were taken as a whole to evaluate and optimize the potential formulation design space for all the design parameters summarized in Table 1. FIG. 14. shows a thermoresponsive gel formulation design space (white area) with limits of 27 to 32° C. for gel temperature. This design space can represent a scenario where the dose is prepared at 2-8° C. and administered while still chilled or refrigerated. Limits: 15-90 s gel time, viscosity at 5° C.≤183 mPas, injection duration=10 s, and >220 μm needle ID). FIG. 15. further narrows the design space with the same factors with an additional parameter constraint for viscosity at 20° C. of ≤183 mPas.

TABLE 4
DESIGN OF EXPERIMENTS STUDY OF GEL COMPOSITION IMPACT
ON GELATION TEMPERATURE, TIME, AND VISCOSITY.

| | | | | Gelation | Gelation | Increase in | | |
|---|---|---|---|---|---|---|---|---|
| | | | | Time for | Time for | Gelation Time | | |
| | Poloxamer | Poloxamer | Gel | 20° C. to | 5° C. to | Initially at 5° C. | Viscosity | Viscosity |
| DOE | 407 | 188 | Temperature | 34° C. Jump | 34° C. | compared to | at 20° C. | at 5° C. |
| Pattern | (% w/v) | (% w/v) | (° C.) | (s) | Jump (s) | 20° C.^{c} (s) | (mPas) | (mPas) |
| −− | 16 | 0 | 23.72 | 9.92 | 29.87 | 20.0 | 54.6 | 27.6 |
| −+ | 22 | 0 | 18.85 | 1^{a} | 20.53 | 19.5 | NA^{b} | 53.8 |
| +− | 16 | 16 | 28.75 | 20.05 | 43.27 | 23.2 | 233.4 | 145.7 |
| ++ | 22 | 16 | 21.93 | 3.42 | 29.71 | 26.3 | NA^{b} | 296.9 |
| a0 | 19 | 0 | 22.04 | 5.58 | 24.85 | 19.3 | 164.1 | 36.5 |
| A0 | 19 | 16 | 24.03 | 13.34 | 33.47 | 20.1 | 497.1 | 203 |
| 0a | 16 | 8 | 33.88 | 34.52 | 78.76 | 44.2 | 85.7 | 59.4 |
| 0A | 22 | 8 | 25.9 | 10.13 | 31.46 | 21.3 | 330.4 | 92.9 |
| 0 | 19 | 8 | 28.89 | 14.45 | 39.30 | 24.9 | 203.1 | 82.2 |
| 0 | 19 | 8 | 29.03 | 17.82 | 38.92 | 21.1 | 200 | 82.6 |

^{a}gelled immediately, so 1 s entered as result placeholder.
^{b}NA, already had gelled.
^{c}average increase in gelation time from 5° C. to 34° C. compared to 20° C. to 34° C. is 24 ± 7 s

### 5.1.5 Rheological Evaluation of Formulations A, B and C within the Design Space

Based on the DOE design space study results, three formulations, A, B, and C shown as crosshairs on FIG. 15. were identified for additional study. The compositions are: A=6%/19%, B=6.5%/18%, and C=7%/17.5% w/v poloxamer 188 and 407, respectively.

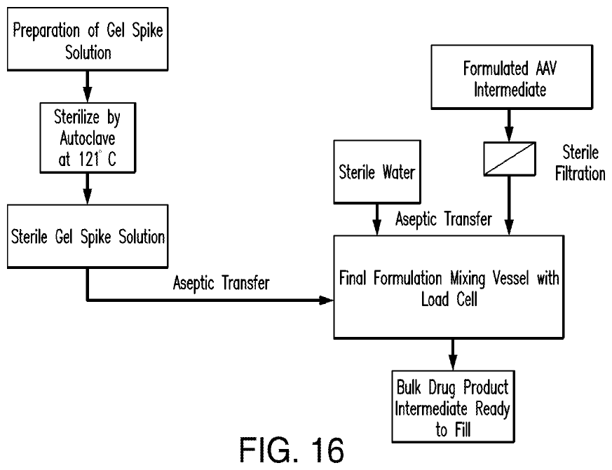

Formulations A, B and C were prepared sterile as shown in FIG. 16. All formulations were based on modified Dulbecco's phosphate-buffered saline solution. Modified Dulbecco's phosphate-buffered saline with sucrose solution was used as a control (100 mM sodium chloride, 2.70 mM potassium chloride, 8.10 mM sodium phosphate dibasic anhydrous, 1.47 mM potassium phosphate monobasic, 117 mM sucrose, 0.001% (0.01 mg/mL) poloxamer 188). The spike solutions were prepared slightly more concentrated by a ratio of 10/9 or 1.11-fold so that they can be spiked at a ratio of 9/10 with a ratio of 1/10 of the AAV intermediate to achieve the desired final composition, for example. Other dilution ratios could also be used. Viscous formulations can be difficult to sterile filter, so they were sterilized by autoclave at 121° C. for 20 min (suitable for up to 200 mL; longer times may be used for larger volumes, such as 40 min for 2000 mL). A ‘liquids’ cycle was used that gradually reduces the pressure when the temperature is reduced to prevent ‘boiling-over’ and a bottle with a cap equipped with a sterile filter was used to allow the steam to enter the bottle while maintaining sterility for then transferred to a sterile hood for filling (example caps include Chemglass CLS1484-12, or Sartorius MYCAP™ series of caps or equivalent). Executed cycles indicated that between 2 to 4% of mass may be lost due to evaporation of water during the cycle and this may be added back in as sterile water for injection along with spiking of the active AAV formulated intermediate. Table 7 shows that there was no impact of autoclaving on the thermal gelling properties of the formulations. Slight differences in the values represent measurement and preparation variability.

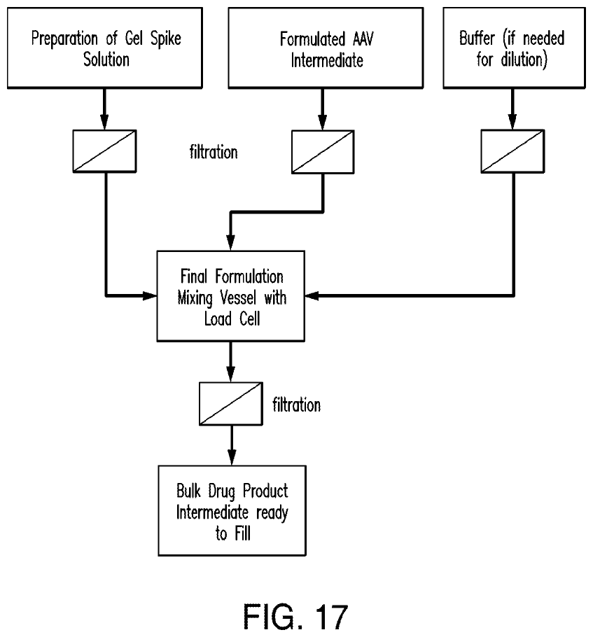

An alternative preparation shown in FIG. 17. involves sterile filtration. With optimization of the formulation viscosity by composition and temperature and optimization of the filtration flow and pressure, sterile filtration may be also feasible for sterilization of the final product.

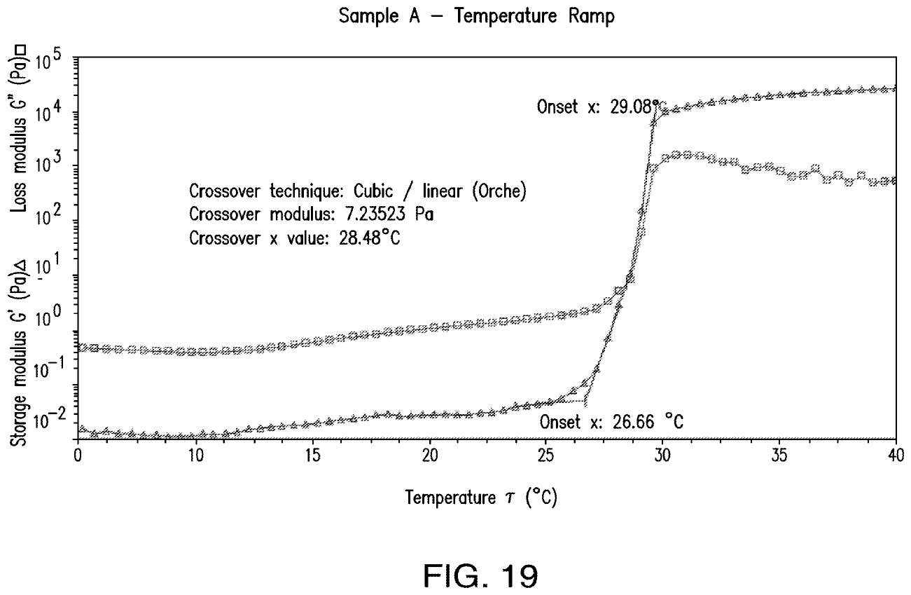

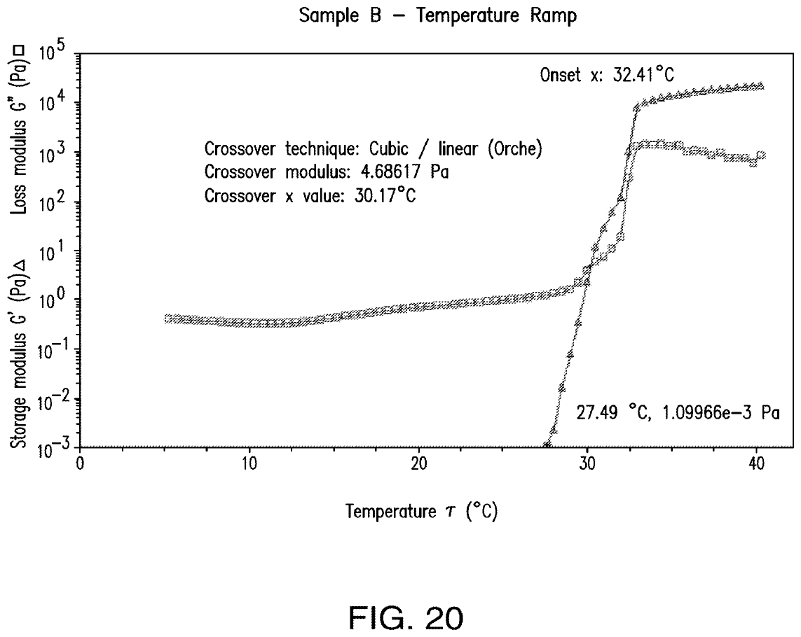

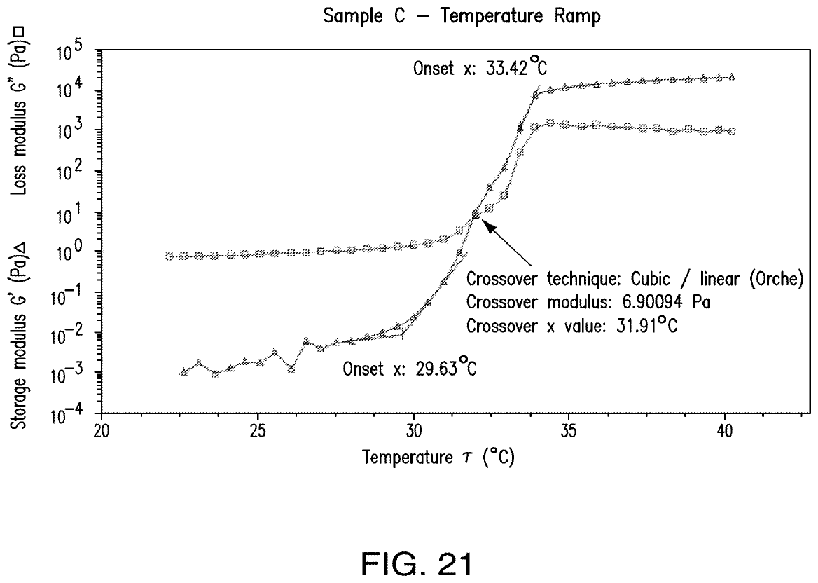

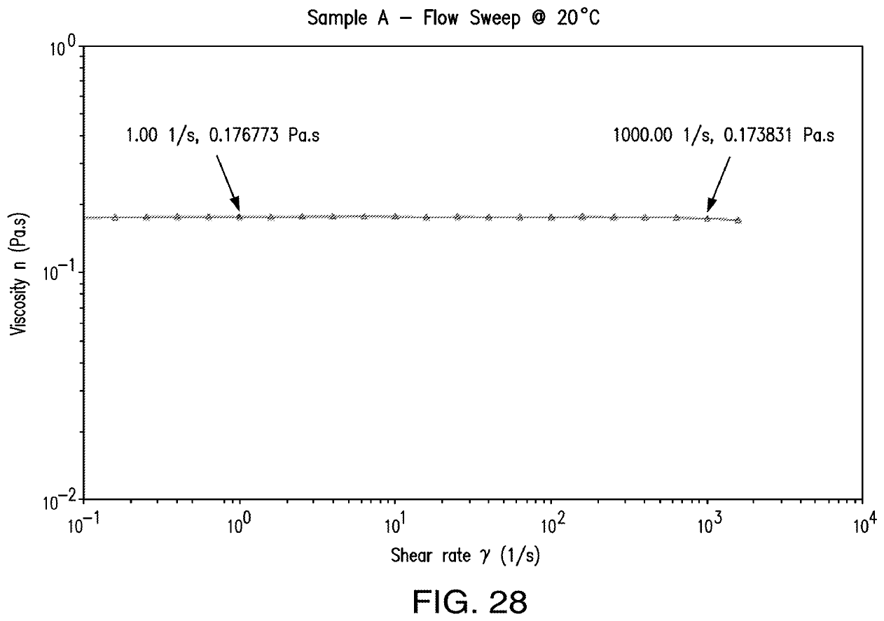

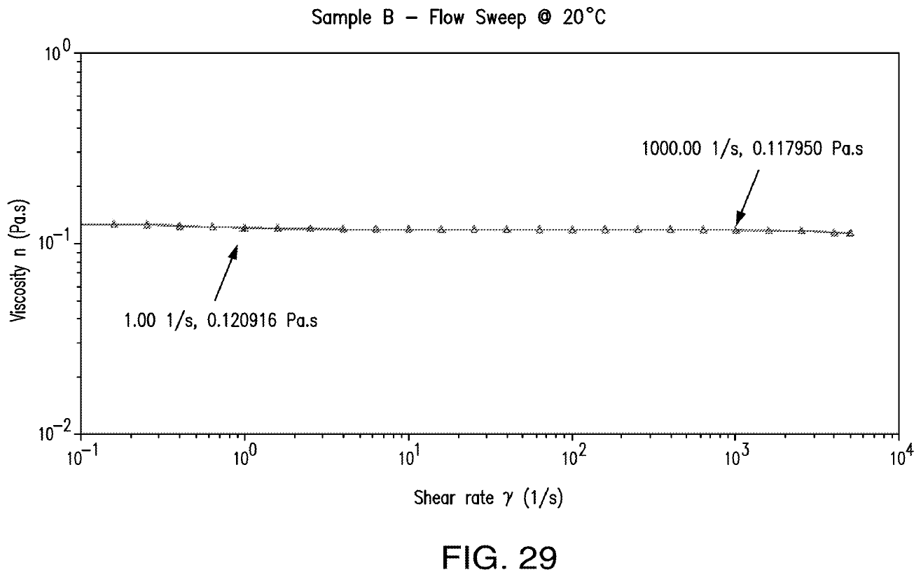

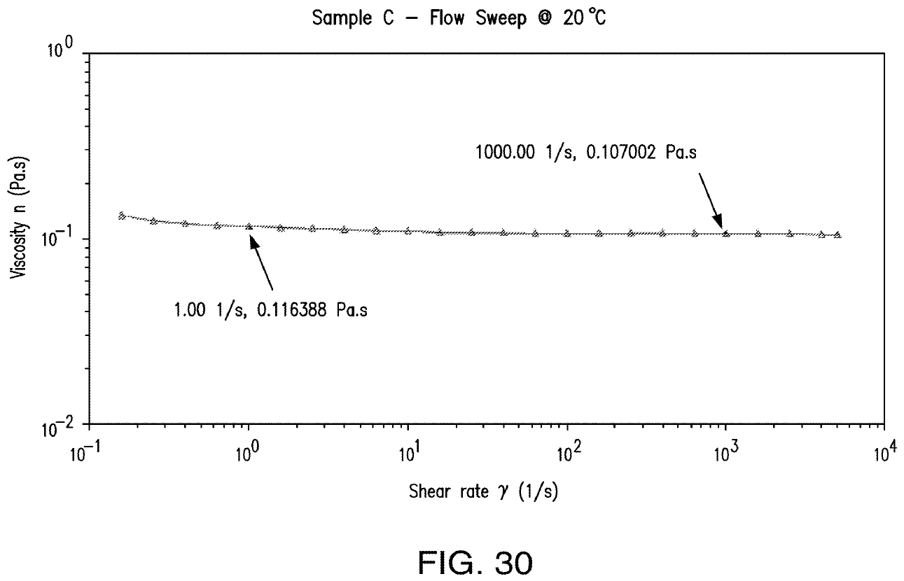

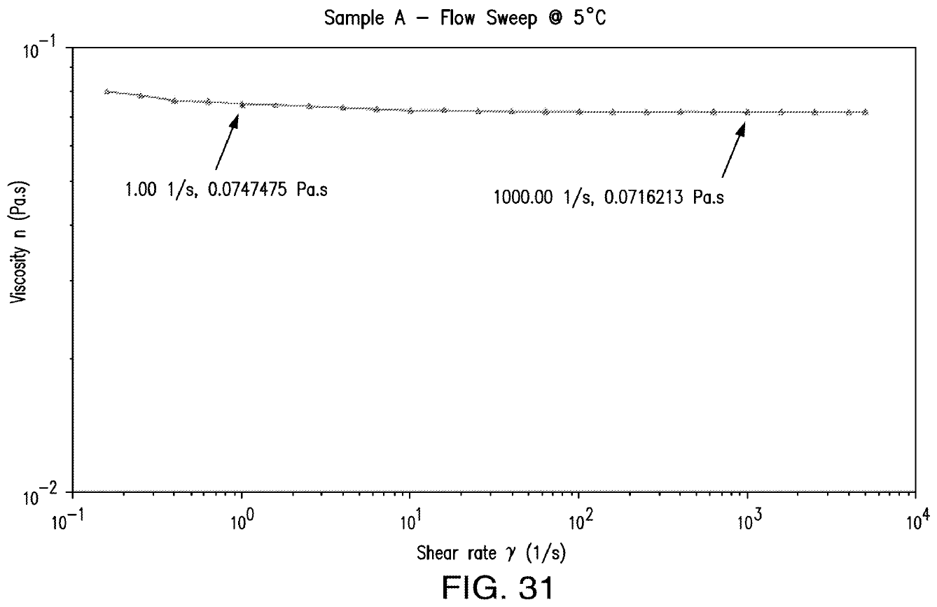

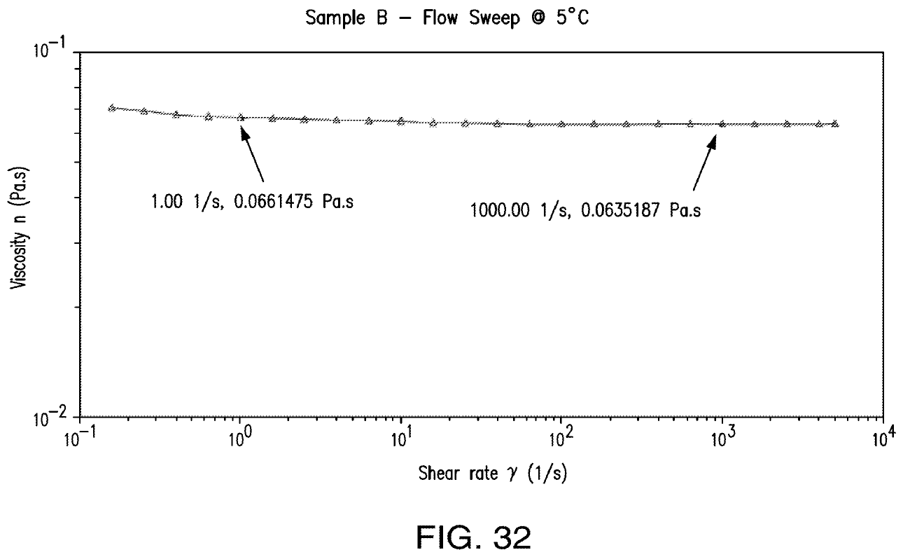

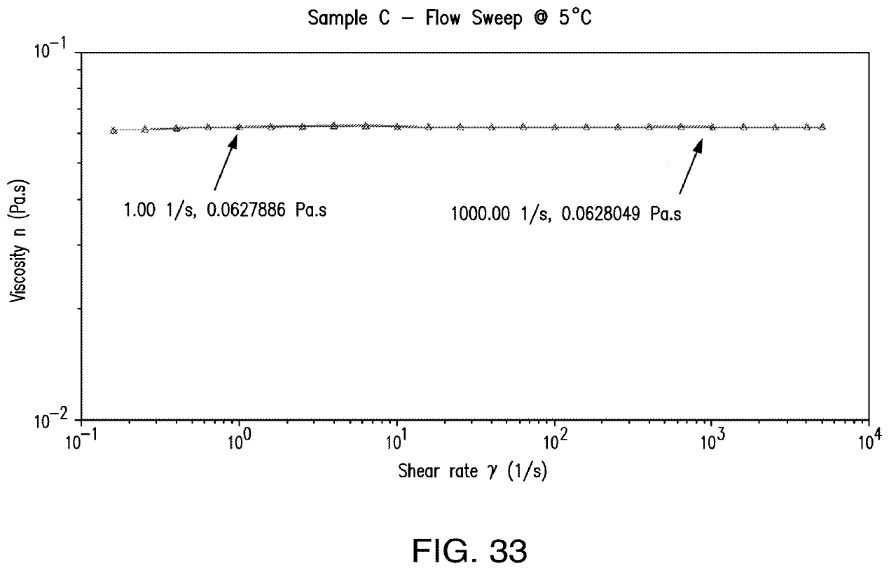

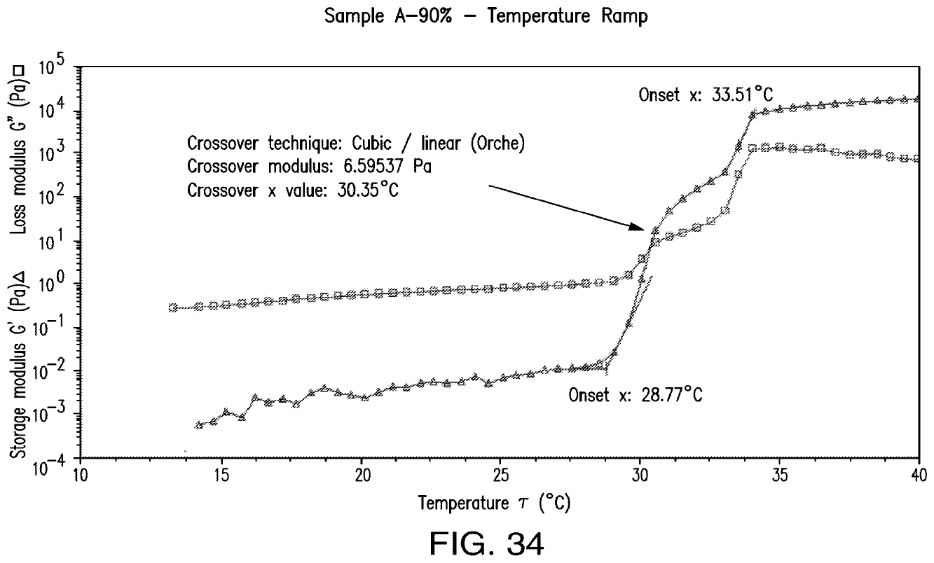

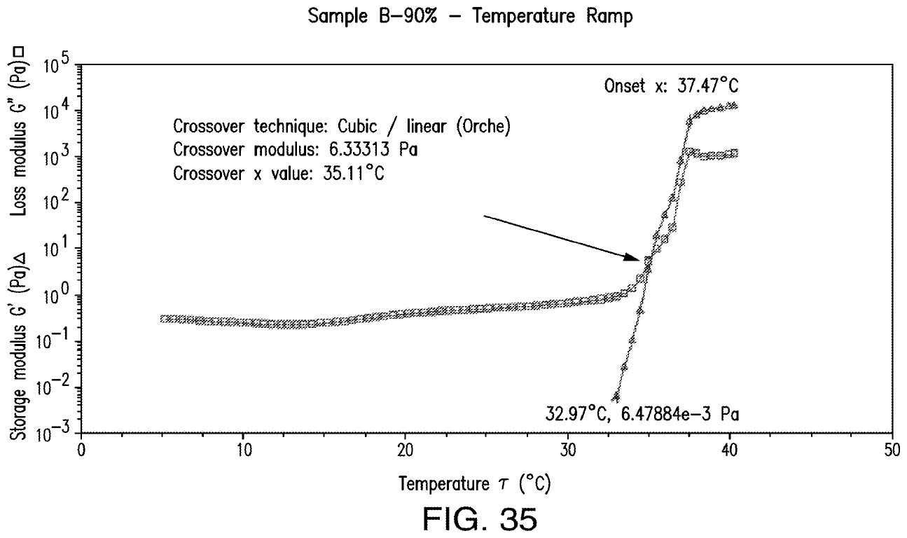

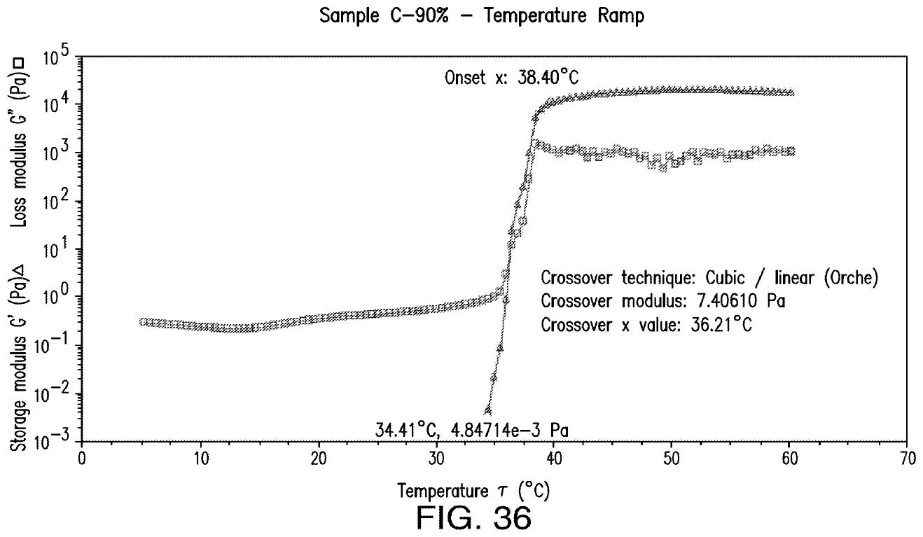

Table 5 summarizes the rheological properties of Formulations A, B, and C. The gelation temperatures, ranged from 28° C. to 32° C. The onset for gelation is also shown and ranged from 27 to 300 and the plateau for compete gelation ranged from 29° C. to 33° C. These all fall within the design space for gelation to occur at or below the temperature of the eye (˜34.5° C.). The time to gel ranged from 16 to 29 s for the three formulations, also within the initial design parameters. Finally, the viscosity of the three formulations were ≤183 mPas at 20° C., also within the design parameters. The profiles for gelation of the formulations A, B, and C are shown in FIG. 19, FIG. 20, and FIG. 21. The onset of gelation occurs about 2° C. below the crossover point and the change in G′ over the entire gelation covers 6 to 7 orders of magnitude increase in elastic modulus. The viscosity profiles are 20° C. and 5° C. are shown in FIG. 28 to FIG. 33.

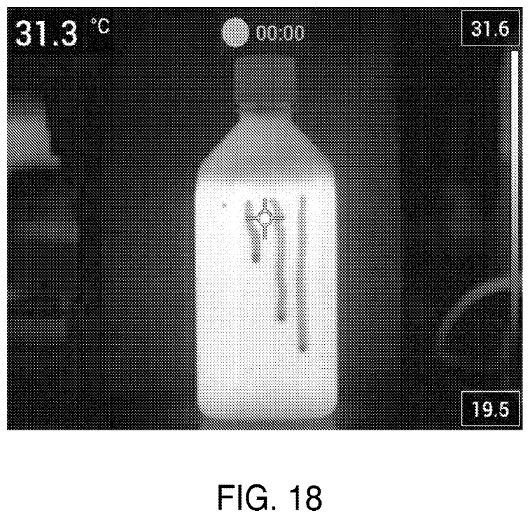

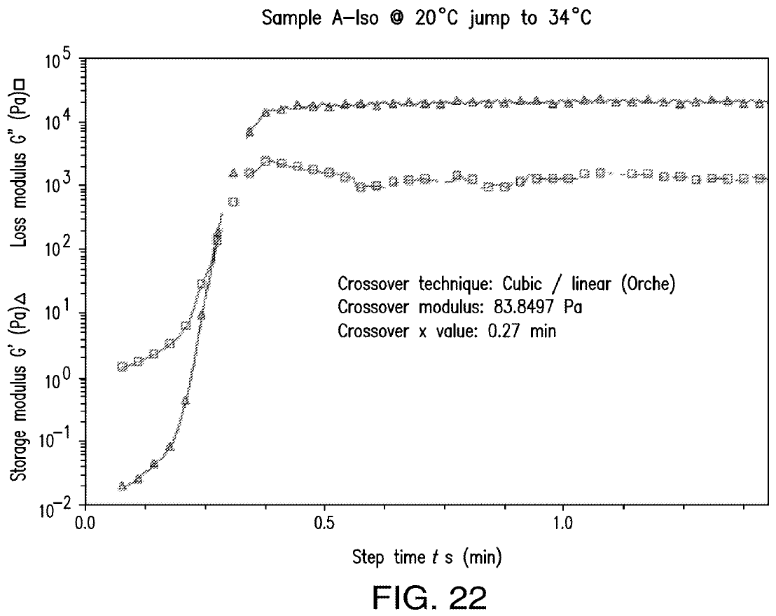

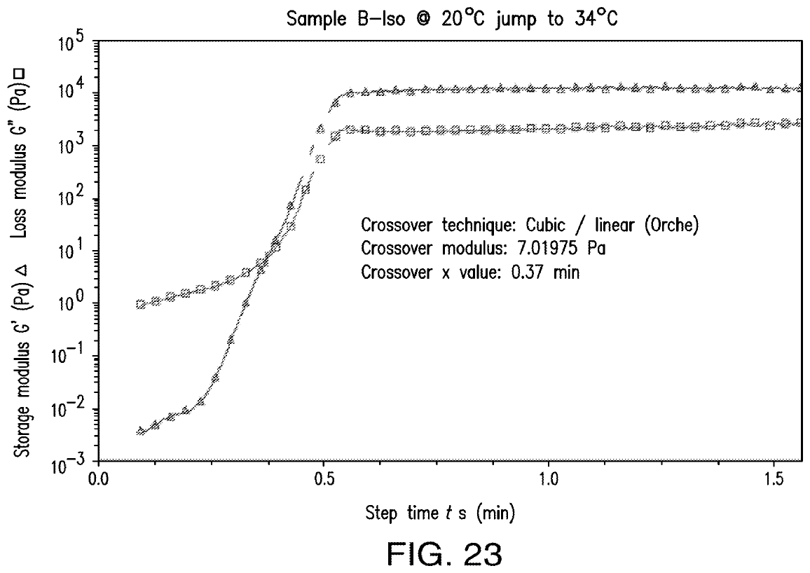

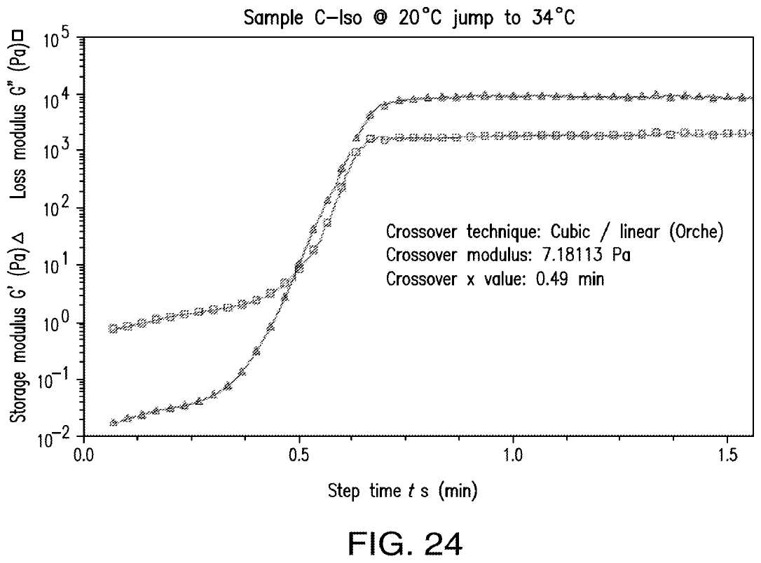

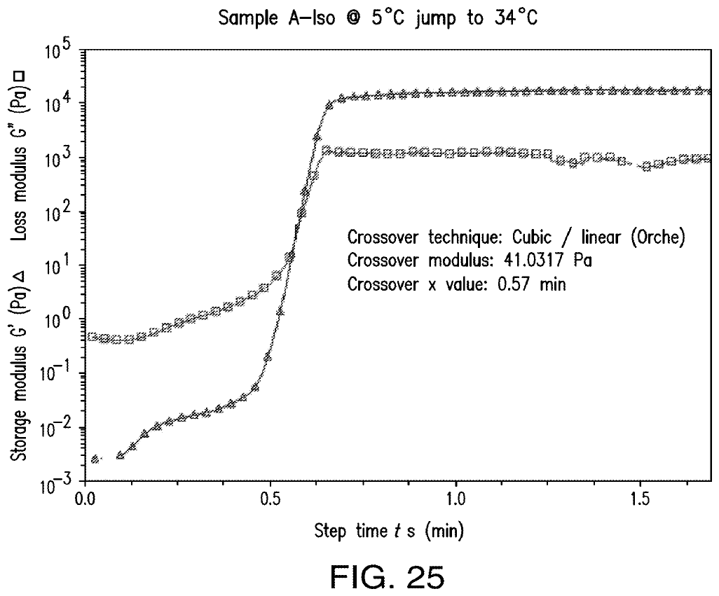

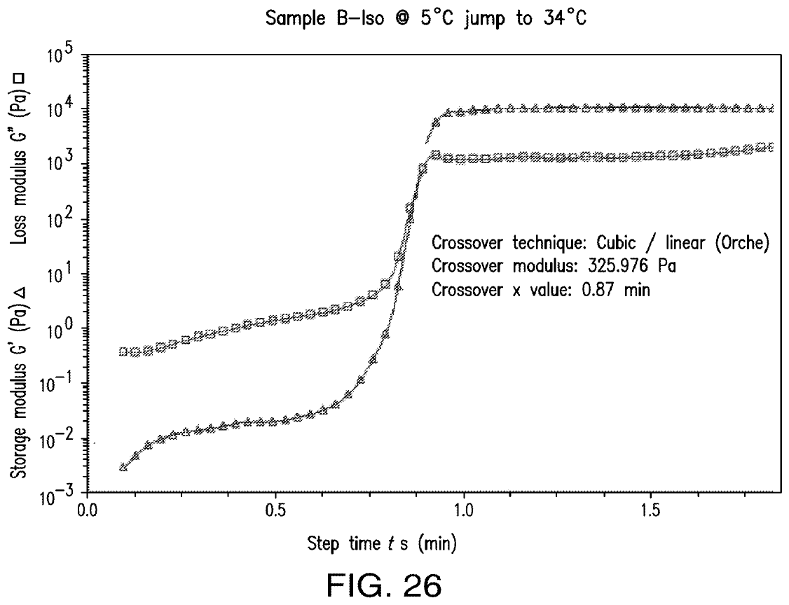

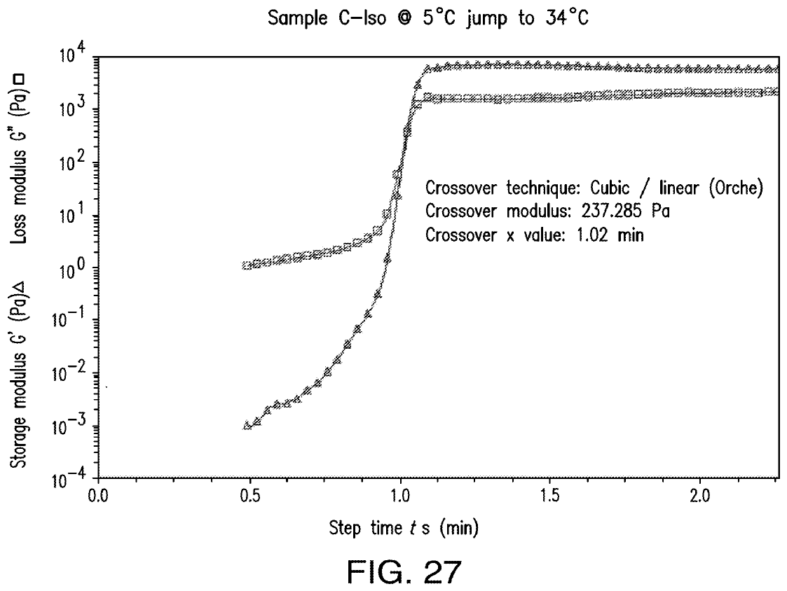

FIG. 18. shows a different method of assessing gelation time. A 50 μL volume of formulation A (left), B (middle) and C (right) at 20° C. were dispensed on the warm surface and a video of the flow of the droplet used to determine the time that the droplet stopped flowing. The times to gel using this approach were about 10 s (A), 25 s (B), and 45 s (C). The rheology profiles for gelation time starting at 20° C. and 5° C. with a jump to 34° C. are shown in FIG. 22 through to FIG. 27.

It is possible that Formulations may be slightly diluted after injection before they become fully gelled, especially at the periphery of the bolus injection. Formulations A, B, and C were placed in a way towards the upper-right of the design space that will allow for a dilution (slight down and left movement in design space) along the isothermal lines for gelation (see FIG. 15). Therefore, the formulations gelation properties will be robust and maintained if there is some slight dilution after injection. The main integrity of the bolus for these formulations is expected to be maintained given how rapidly they will gel. The impact of a 10% dilution by mass using Dulbecco's phosphate buffered saline with 0.001% P188 to mimic a slight dilution by in vivo fluid was performed and the rheological properties measured. At the crossover point used to define the gelation temperature, the storage modulus G′ is already increased substantially compared to the behavior at room temperature. The gelation onset was acceptable for all three formulations after dilution by 10% (Table 6) and this is expected to maintain the integrity of the gel periphery immediately after injection before the main bolus can fully gel. Even with a slight shift in the gelation properties for diluted formulations, the G′ value is increased by at least an order of magnitude at the temperature of the eye. The gelation time after slight dilution increased but remained within 90 s for formulations A and B and within 4.4 min for formulations C; all within the limit of 10 minutes for the initial design. These data indicate that the formulations will all be stable with respect to a slight dilution at the periphery immediately after injection before gelation of the bolus can substantially complete within the first 15 to 30 s after injection.

TABLE 5
RHEOLOGICAL AND THERMAL PROPERTIES OF FORMULATIONS A, B, AND C

| | | | | Gelation | Gelation | | |
|---|---|---|---|---|---|---|---|
| | | | | Time for | Time for | | |
| | Poloxamer | Poloxamer | | 20° C. to | 5° C. to | Viscosity | Viscosity |
| | 188 | 407 | | 34° C. | 34° C. | at 20° C. | at 5° C. |
| Formulation | (% w/v) | (% w/v) | Gel Temperature (° C.) | Jump (s) | Jump (s) | (mPas) | (mPas) |
| A | 6 | 19 | onset = 26.66 | 16.2 | 34.2 | 174 | 177 |
| | | | crossover = 28.45 | | | | |
| | | | plateau = 29.08 | | | | |
| B | 6.5 | 18 | onset = 27.49 | 22.2 | 52.2 | 118 | 121 |
| | | | crossover = 30.17 | | | | |
| | | | plateau = 32.41 | | | | |
| C | 7 | 17.5 | onset = 29.63 | 29.4 | 61.2 | 107 | 116 |
| | | | crossover = 31.91 | | | | |
| | | | plateau = 33.42 | | | | |

TABLE 6
IMPACT OF DILUTION ON THE GEL TEMPERATURE
AND GEL TIME FOR FORMULATIONS A, B, AND C

| | | | Gelation Time | |
|---|---|---|---|---|
| | Gel Temperature (° C.) | | at 20° C. (s) | |
| Formulation* | Neat | 90% | Neat | 90% |
| A | onset = 26.66 | onset = 28.77 | 16.2 | 28.2 |
| | crossover = 28.45 | crossover = 30.35 | | |
| | plateau = 29.08 | plateau = 33.51 | | |
| B | onset = 27.49 | onset = 32.97 | 22.2 | 60.6 |
| | crossover = 30.17 | crossover = 35.11 | | |
| | plateau = 32.41 | plateau = 37.47 | | |
| C | onset = 29.63 | onset = 34.41 | 29.4 | 262.8 |
| | crossover = 31.91 | crossover = 36.21 | | |
| | plateau = 33.42 | plateau = 38.40 | | |

TABLE 7
IMPACT OF AUTOCLAVE STERILIZATION
ON THE GEL TEMPERATURE AND TIME

| | | | Gelation Time | |
|---|---|---|---|---|
| | Gel Temperature (° C.) | | at 20° C. (s) | |
| Formu- | | Non- | | Non- |
| lation* | Autoclaved | autoclaved | Autoclaved | autoclaved |
| A | onset = 26.66 | onset = 26.56 | 16.2 | 16.2 |
| | crossover = 28.45 | crossover = 28.29 | | |
| | plateau = 29.08 | plateau = 29.99 | | |
| B | onset = 27.49 | onset = 28.51 | 22.2 | 27.0 |
| | crossover = 30.17 | crossover = 30.66 | | |
| | plateau = 32.41 | plateau = 32.45 | | |
| C | onset = 29.63 | onset = 29.01 | 29.4 | 28.2 |
| | crossover = 31.91 | crossover = 32.03 | | |
| | plateau = 33.42 | plateau = 33.45 | | |

### 5.1.6 Stress Stability of Construct II in Formulations A, B and C

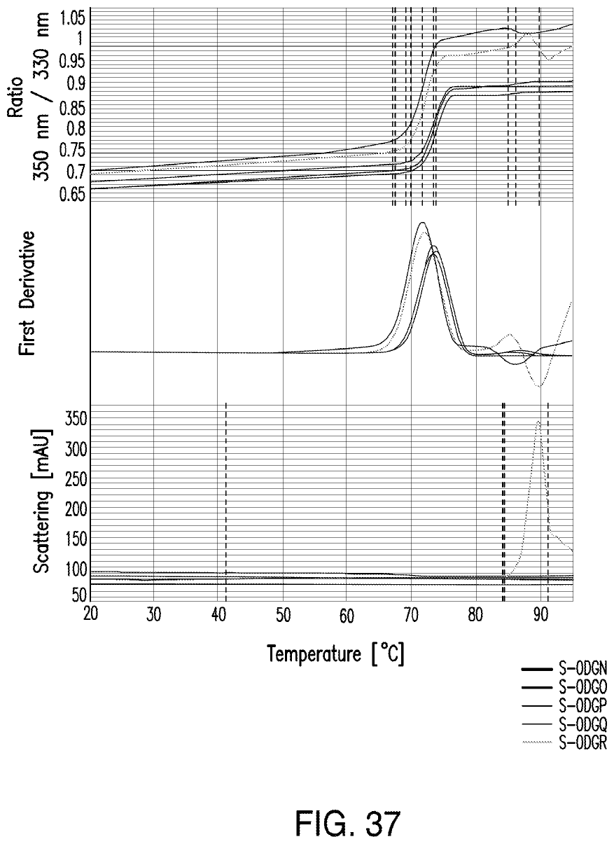

Differential scanning fluorimetry (DSF) measures the intrinsic tryptophan and tyrosine emission of proteins as a function of temperature. The local environment of Trp and Tyr residues changes as the protein unfolds resulting in a large increase in fluorescence. FIG. 37. shows differential scanning fluorimetry thermal ramp data for a control compared to formulations A, B, and C. Top panel: raw melting curve signal. Middle panel: derivative of data to identify the peak. Bottom panel: light scattering data to indicate either aggregation. All the formulations had similar profiles to the control upon thermal ramping. Table 8 summarizes the results that the formulations A, B and C have a similar thermal stability to the control formulation and that the AAV is therefore stable.

TABLE 8
DIFFERENTIAL SCANNING FLUOROMETRY MELTING TEMPERATURE
OF THE CONSTRUCT II AAV CAPSID IN THE DIFFERENT
GEL FORMULATIONS COMPARED TO THE CONTROL

| | Control in | Formulation A | Formulation B | Formulation C |
|---|---|---|---|---|
| | modified DPBS | (19% P407/ | (18% P407/ | (17.5% P407/ |
| Test | with Sucrose | 6% P188) | 6.5% P188) | 7% P188) |
| Sample ID | S-0DGN | S-0DGO | S-0DGP | S-0DGQ |
| Differential Scanning | 71.69 | 73.36 | 73.81 | 73.46 |
| Fluorometry Melting | | | | |
| Temperature (° C.) | | | | |

Table 9 summarizes the impact of 37° C. stress on the stability of AAV in formulations A, B and C compared to a control. The free DNA detected, which is a measure of AAV instability resulting in capsid disruption with release of the DNA payload was low and similar to the initial level accounting for method variability for all samples after 3 days at 37° C. The vector genome concentration (by ddPCR) of the formulations A, B, and C was measured on samples diluted 3-fold to reduce the viscosity. The results had some variability, likely related to the fact they were diluted by volume rather than by mass (since they have high viscosity the dilution may have introduced variability). The 3 day formulation A and B samples had similar vector genome concentrations to the control and overall indicate they were stable. The variability in dilution can be seen in the initial sample of formulation B being higher than the target and the 3 day sample of formulation C being lower. In future, dilution by mass accounting for density is an approach that could potentially help reduce variability in sample preparation. Overall, vector genome concentrations of A, B, and C were similar to the to the control, when accounting for variability, indicating that the formulations all have a similar and acceptable stability with respect to temperature stress.

TABLE 9
IMPACT OF 37° C. STRESS ON THE STABILITY
OF FORMULATIONS A, B AND C COMPARED TO A CONTROL

| | Time | Control in | Formulation A | Formulation B | Formulation C |
|---|---|---|---|---|---|
| | Point | modified DPBS | (19% P407/ | (18% P407/ | (17.5% P407/ |
| Test | (days) | with Sucrose | 6% P188) | 6.5% P188) | 7% P188) |
| Vector Genome | 0 | 1.41 × 10¹² | NT | 1.85 × 10¹² | NT |
| Concentration | 3 | 1.36 × 10¹² | 1.57 × 10¹² | 1.18 × 10¹² | 5.79 × 10¹¹ |
| (ddPCR) | | | | | |
| (GC/mL) | | | | | |
| Free DNA (%) | 0 | 1.2 ± 0.5 | 0.5 ± 0.3 | 1.2 ± 0.7 | 0.6 ± 0.3 |
| by SYBR Gold | 3 | 1.7 ± 0.8 | 0.8 ± 0.6 | 1.3 ± 0.7 | 2.8 ± 1.2 |

### 5.1.7 Evaluation of Injectability and Dose Preparation of Formulations A, B and C

Samples of A, B and C filled into 2 mL COP (West Pharmaceuticals, 19550057) Crystal Zenith™ vials and extracted using a vial adapter (West, 8070117). All samples were easily filled into the syringe at room temperature as assessed on multiple days. The room temperature was measured to be 22.4° C.±0.6° C. (min=20.7° C. and max=23.7° C.) over a representative 1 week period. This demonstrates that the gelation properties allow for handling at the typical controlled room temperature.

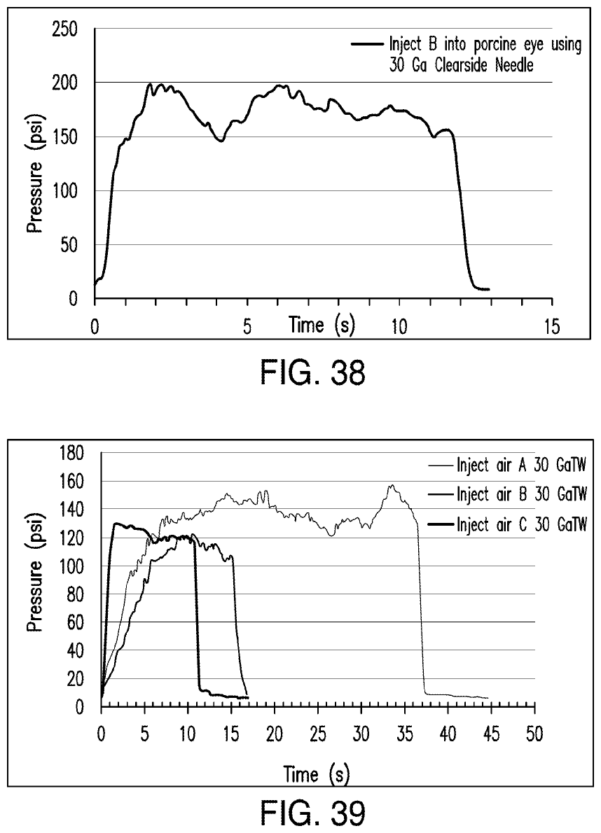

FIG. 38 shows the injection pressure for injection of formulation B into an enucleated porcine eye equilibrated at 35° C. using a Clearside syringe device (CLS-HN001) and 30 gauge (160 μm ID, CLS-MN1100) needle. The formulation was easily injected into the eye, although the pressure was about 160 PSI. However, scaling the needle ID used (160 μm ID) to a larger need ID will result in about 5-times lower pressure based on the Hagen-Poiseuille equation as pressure is inversely proportional to inner diameter to the fourth power (i.e. (240/160)⁴=5). Therefore with the needle in development, the pressure expected is 5-time lower or about 32 PSI, well within the acceptable range.

FIG. 39. shows injection into air using a BD 1 mL syringe (309628) with 30 GG*½ inch (0.3*13 mm) TW needle (Nipro, HN-3013-ET). The Clearside device and needle are designed with a specific exposed needle length for suprachoroidal injection. Currently the 30 Gauge (160 μm ID) is available in this format. Larger needle ID versions with 220 or 240 μm ID are in development. Injection using known needle sizes can be scaled according to the relationship of pressure to injection time and needle ID given by the Hagen-Poiseuille equation. Therefore injection with available size needles can be used to identify if the formulations will have the desired injection pressure and time parameters for needles currently under manufacturing development. The pressure scaling for this needle relative to a larger ID needle is about 4.5-fold (240/165)⁴=4.5). Pressure is also proportional to flow rate. In this experiment the pressure was held constant and the time to inject evaluated. The pressure and time to inject can then be scaled by a total combined factor of 4.5 to translate the readings to the larger needle in development. Therefore, formulation C was injected over 12 s with about 120 PSI with the 30 GaTW (165 μm ID) needle, which will reduce to about 27 PSI if a 240 μm ID needle is used. Formulation B was injected over 17 s with about 120 PSI, which will also reduce to about 27 PSI with a larger needle. If formulation B is injected more quickly, such as over 12 s, then a larger need ID pressure of about 38 PSI is expected. These are well within the injection pressure design space identified. If the 37 s and 140 PSI injection of formulation A is scaled to a larger needle and faster injection, then the expected pressure for a 12 to 16 s injection is 64 to 78 PSI, also within the limiting design space that was identified (<100 PSI or <64 PSI).

### (a) Rheology Measurements

Rheometry measurements are described in the United States Pharmacopeia (USP) USP<1911> and rotational viscometry is described in USP<912>.

Rheology measurements were collected with an AR-G2 rheometer equipped with a Peltier temperature control plate with a 60 mm 1° angle aluminum cone accessory (TA Instruments, New Castle, DE).

### (b) Gelation Temperature

The gelation temperature was determined using by applying at temperature ramp in oscillatory mode at 0.1% strain and 1 Hz. The samples were loaded and pre-equilibrated for 5 minutes at 5° C., followed by a temperature ramp at 5° C./min up to either 40° C. or 60° C. The temperature at which the storage/elastic modulus (G″) and loss/viscous modulus (G′) crossed was recorded as the system gelation temperature. A torque sweep demonstrated that linear viscoelastic region extended to about 0.4% and therefore operation at 0.1% was well-within the linear viscoelastic region.

### (c) Gelation Time

Gelation time was determined in oscillatory mode 0.1% strain and 1 Hz. Samples equilibrated at both 5° C. and 20° C. and exposed to a temperature jump to 34° C. As above, the time to gel was defined as the crossover of the storage and loss modulus curves.

### (d) Viscosity Versus Shear Rate

A viscosity versus shear rate sweep was performed over the range 0.25 s⁻¹ to 5000 s⁻¹ with 5 points per decade collected. The viscosity versus shear rate was collected at both 5° C. and 20° C. Viscosity at 10,000 and 20,000 s⁻¹ were extrapolated from the data.

### (e) Differential Scanning Fluorimetry

The thermal stability of proteins and virus capsids made up of proteins can be determined by differential scanning fluorimetry (DSF). DSF measures the intrinsic tryptophan and tyrosine emission of proteins as a function of temperature. The local environment of Trp and Tyr residues changes as the protein unfolds resulting in a large increase in fluorescence. The temperature where 50% of proteins are unfolded is defined as the ‘melting’ temperature (Tₘ). Fluorescence spectroscopy is described in the USP<853> and USP<1853>.

DSF data was collected using a Promethius NT.Plex Nano DSF Instrument (NanoTemper technologies, Munich, Germany). Samples were loaded into the capillary cell at 20° C. and the temperature ramped at a rate of 1° C./min. to 95° C. The signal output ratio of emission at 350 nm (unfolded) and 330 nm (unfolded) was used to determine the Tₘ.

### (f) Injection Pressure Measurements

Injection pressures were measured using either a Flow Screen and Fluid Sensor (Viscotec America, Kennesaw, GA) or a PressureMAT-DPG with pressure sensor S-N-000 (PendoTECH, Princeton, NJ).

Injections into air were either performed manually or using a Legato-100 syringe pump (Kd Scientific, Holliston, MA) to apply a consistent flow rate. For injections into enucleated porcine eyes, the eyes were mounted on a Mandell eye mount (Mastel, Rapid City, SD) with applied suction to adjust the intraocular pressure of the eye.

### 5.2 Example 2: Pharmacodynamic, Biodistribution, and Tolerability Study in Cynomolgus Monkeys Using Different Suprachoroidal Formulations

The objective of this study is to evaluate the biodistribution, pharmacodynamics (transgene concentration), and tolerability of different formulations comprising AAV8-anti-VEGF-ab when administered as a single dose via suprachoroidal injection to Cynomolgus monkeys. After dosing, animals are observed postdose for at least 4 weeks. One group is also administered a high volume of the formulations. Some of the formulations include varying gel temperatures. For example, Formulation 1 has a gel temperature of about 28° C., Formulation 2 has a gel temperature of about 30° C., and Formulation 3 has a gel temperature of about 32° C. The group assignment and dose levels are shown in Table 10. The test article is AAV8-anti-VEGF-ab. The control article is a placebo. The formulations and the controls can be stored in a freezer between −60° C. and −80° C. and thawed at room temperature on the day of use, or stored at room temperature if used on the day of formulation, or stored in a refrigerator between 2° C. and 8° C. The indication is chronic retinal conditions including wet AMD and diabetic retinopathy.

TABLE 10
GROUP ASSIGNMENT AND DOSE LEVELS

| | | | | Dose | |
|---|---|---|---|---|---|
| | Dose | Dose | Dose | Concen- | Number of |
| | Regimen | Regimen | level^{b} | tration | Animals |
| Group | Left Eye | Right Eye | (GC/eye) | (GC/mL) | (Females) |
| Control 1^{a} | Control | Control | 0 | 0 | 41 |
| | Article 1 | Article 1 | | | |
| Control 2 | Control | Control | 0 | 0 | 1 |
| | Article 2 | Article 2 | | | |
| Control 3 | Control | Control | 0 | 0 | 1 |
| | Article 3 | Article 3 | | | |
| Formulation | Test | Test | 3 × | 3 × | 4 |
| 1 | Article 1 | Article 1 | 10¹¹ | 10¹² | |
| Formulation | Test | Test | 3 × | 3 × | 4 |
| 2 | Article 2 | Article 2 | 10¹¹ | 10¹² | |
| Formulation | Test | Test | 3 × | 3 × | 4 |
| 3 | Article 3 | Article 3 | 10¹¹ | 10¹² | |
| High | Test | Test | 3 × | 1.5 × | 4 |
| volume | Articles 1, | Article 1, | 10¹¹ | 10¹² | |
| formulation | 2, or 3 | 2, or 3 | | | |

GC = Genome copies
^{a}Group 1 will be administered control article only.
^{b}Dose levels are based on a dose volume of 100 μL/eye for Formulations 1-3, and volume of 200 μL/eye for the high volume formulation group. Each eye is administered two injections.
c all animals are sacrificed on day 29 of the dosing phase.

Antibody Prescreening at Animal Supplier: blood (at least 1 mL) from about 90 female monkeys is collected from each animal via a femoral vein and placed into tubes containing no anticoagulant. Another vein may be used for collection, as needed. Animals are selected as study candidates based on the pre-screening results. Blood is allowed to clot at room temperature and centrifuged within 1 hour to obtain serum. Serum is divided into 2 aliquots and placed into cryovials and maintained on dry ice prior to storage at approximately −70° C. Samples are shipped overnight on dry ice for analysis. Samples are then analyzed for anti-AAV8 neutralizing antibodies (NAbs) by any acceptable method. Animals are selected for shipment based on anti-AAV8 Nab results.

Dose Administration: animals are fasted overnight and anesthetized with ketamine and dexmedetomidine prior to suprachoroidal injection. In brief, a single suprachoroidal injection of 100 μL (or 2 injections of 50 μL each) is administered to each eye (between 3 and 4 mm from the limbus) over 5 to 10 seconds. The formulations are all administered at room temperature. The formulations are administered with Clearside SCS Microinjectors. The microneedle size varies depending on the viscosity of the formulation. In some cases a 30-gauge microneedle is used. Injections in the right eye are administered in the superior temporal quadrant (i.e., between the 10 o'clock and 11 o'clock positions. Injections in the left eye are administered in the superior temporal quadrant (i.e., between the 1 o'clock and 2 o'clock positions). Following the injection, the needle is kept in the eye for approximately 5 seconds before being withdrawn. Upon withdrawal of the micro needle, a cotton-tipped applicator (dose wipe) is placed over the injection site for approximately 10 seconds. A topical antibiotic (e.g. Tobrex® or appropriate substitute) is instilled in each eye following dosing. Each dosing time is recorded as the time at the completion of each injection. The right eye is dosed first, followed by the left eye.

Ophthalmic Procedures: ophthalmic examinations (e.g., on days 4, 8, 15, and 29 post administration) are conducted. Animals are examined with a slit lamp biomicroscope and indirect ophthalmoscope. The adnexa and anterior portion of both eyes are examined using a slit lamp biomicroscope. The ocular fundus of both eyes are examined (where visible) using an indirect ophthalmoscope. Prior to examination with the indirect ophthalmoscope, pupils are dilated with a mydriatic agent (e.g., 1% tropicamide). Intraocular pressure is measured on the day of administration (within 10 minutes prior to dosing) and, for example, on days 4, 8, 15, and 29. Rebound tonometry (TonoVet) can be used to evaluate ocular pressure. Ocular photography is performed around week 4. Photographs are taken with a digital fundus camera. Color photographs are taken of each eye to include stereoscopic photographs of the posterior pole and nonstereoscopic photographs of two midperipheral fields (temporal and nasal). Photographs of the periphery is also performed. Further, autofluorescence imaging with indocyanine green is conducted to document spread of dose (e.g., on days one and two).

Anti-AAV8 Neutralizing Antibody Analysis: blood samples from each animal taken from a femoral vein at different time points (e.g., prior to administration, on day of administration, and on days after administration) are held at room temperature and allowed to clot for at least 30 minutes prior to centrifugation. Samples are centrifuged within 1 hour of collection, and serum is harvested. Following harvesting, samples are placed on dry ice until stored between −60° C. and −80° C. Serum analysis for AAV8 antibodies is then performed using a qualified neutralizing antibody assay.

Anti-AAV8-anti-VEGF-ab Transgene Product Antibody Analysis: blood samples are taken as discussed above and serum samples are analyzed for antibodies to the AAV8-anti-VEGF-ab using any assay of the present disclosure or any acceptable assay. For AAV8-anti-VEGF-ab transgene analysis, blood samples are taken as described above at least two weeks prior to administration, on day 15, and on the day of animal sacrifice (Day 29). 50 μL from the anterior chamber is collected before dose administration. Samples from the aqueous humor and the vitreous humor can be collected at the terminal necropsy. Serum samples can be collected pre-dose, on Day 15, and prior to necropsy. Samples are then analyzed by any assay of the present disclosure or any applicable assay or method (e.g., for transgene concentration).

Aqueous Humor Collection: approximately 50 μL is removed from each eye at least 2 weeks prior to administration, on day 15, and on the day the animals are sacrificed. Aqueous humor samples from each eye is placed into separate tubes with Watson barcoded labels, snap frozen in liquid nitrogen, and placed on dry ice until stored between −60° C. and −80° C.

Post-Aqueous Tap Medication Regimen: the objective of this treatment regimen is to provide palliative treatment related to aqueous humor collection procedures. The treatment objective following collection days is to provide appropriate palliation of adverse events (e.g., discomfort). Animals are tested for ocular pain and side effects.

TABLE 11
MEDICATION REGIMEN

| Days | Drug (Dose Level) | Dose Route | Interval |
|---|---|---|---|
| Day of | Flunixin meglumine | IM | Prior to sedation for |
| sampling | (2 mg/kg) | | collection |
| Day of | Buprenorphine | IM | Upon recovery from |
| sampling | (0.05 mg/kg) | | anesthesia; 5 to 7 |
| | | | hours later, and at |
| | | | least 16 hours later |
| | | | (from the first |
| | | | injection) |
| Day of | 1% Atropine sulfate | Topical | After collection |
| sampling | solution^{a} | | procedures |
| Day of | Neo-Poly-Dex | Topical | After collection |
| sampling | ointment^{b} | | procedures |
| 1 day after | 1% Atropine sulfate | Topical | Once |
| sampling | solution^{a} | | |
| 1 day after | Neo-Poly-Dex | Topical | BID |
| sampling | ointment^{b} | | |
| 2 days after | 1% Atropine sulfate | Topical | Once |
| sampling | solution^{a} | | |
| 2 days after | Neo-Poly-Dex | Topical | BID |
| sampling | ointment^{b} | | |

BID = Twice daily (at least 6 hours apart);
IM = Intramuscular injection
^{a}Applied as 1 to 2 drops of solution to each eye from which samples were collected.
^{b}Applied as an approximate 0.25 inch strip to each eye from which samples were collected.

Termination of Study: animals are anesthetized with sodium pentobarbital and exsanguinated on Day 29.

Necropsy Collections of Aqueous Humor and Vitreous Humor: up to 50 μL per eye and up to 100 μL per eye is removed from the aqueous humor and the vitreous humor, respectively. Following exsanguination, eyes are enucleated and aqueous humor and vitreous humor samples are collected from each eye. Vitreous humor samples are divided into 2 approximately equal aliquots and aqueous humor samples are stored as one aliquot. After each collection, the right eyes of animals are injected with modified Davidson's fixative until turgid. Eyes are stored in modified Davidson's fixative for 48 to 96 hours, and then transferred to 10% neutral-buffered formalin. Samples are flash frozen and stored between −60° C. and −80° C. Aqueous and vitreous samples are analyzed for transgene concentration.

Ocular Tissue Collection for Biodistribution: following exsanguination, the left eye from all animals and right eye from two animals (depending on survival) from the various formulation groups are enucleated and tissues are collected. Tissues are collected into separate tubes with Watson barcoded labels. Collected tissue includes choroid with retinal pigmented epithelium, cornea, iris-ciliary body, optic chiasm, optic nerve, retina, sclera, and posterior eye cup. Eyes are divided into four approximately equal quadrants (superior-temporal to include the area of the dose site, superior-nasal, inferior-temporal, and inferior nasal to include the area of the dose site). From each quadrant, one sample is taken using an 8 mm biopsy punch. Samples are stored between −60° C. and −80° C. Samples are analyzed for vector DNA or RNA using a qPCR or qRT-PCR method.

Non-Ocular Tissue Collection for Biodistribution: two samples of approximately 5 mm×5 mm×5 mm is collected from the right brain hemisphere (e.g., cerebellum (lateral), cerebellum (dorsal), frontal cortex (Brodmann area 4), frontal cortex (Brodmann area 6), occipital cortex (cortical surface), occipital cortex (parenchyma)), ovary, heart, kidney, lacrimal gland (left), liver (left lateral lobe), lung (left caudal lobe), lymph node (parotid), lymph node (mandibular), pituitary gland, salivary gland (mandibular), spleen, thymus, dorsal root ganglia (cervical, left), dorsal root ganglia (lumbar, left), and dorsal root ganglia (thoracic, left). Samples are stored between −60° C. and −80° C.

Histology: right eye and right optic nerve from animals are sectioned at a nominal 5 μm and stained with hematoxylin and eosin. Eye tissues are sectioned to facilitate examination of the fovea, injection site region, macula, optic disc, and optic nerve. A single, vertical section is taken through the approximate center of the inferior calotte. This results in one slide/block/eye (three slides per eye total). Further, digital scans (virtual slides) can be prepared from selected microscopic slides.

Data Evaluation and Statistical Analysis: statistical data analyses are calculated using means and standard deviations. Means and standard deviations are calculated for absolute body weight, body weight change, and intraocular pressure measurements.

### 5.3 Example 3: Pharmacodynamic, Biodistribution, and Tolerability Study in Cynomolgus Monkeys Using Different Suprachoroidal Formulations

The objective of this study was to evaluate the biodistribution (DNA and mRNA), pharmacodynamics (transgene concentration), and tolerability of Gel Formulation B comprising AAV8-anti-VEGF-ab when administered as a single dose via suprachoroidal injection to Cynomolgus monkeys. After dosing, animals were observed postdose for at least 4 weeks. Each group was administered two injections to achieve the same dose volume. The group assignment and dose levels were shown in Table 12. The test article was AAV8-anti-VEGF-ab. The control article was a placebo.

TABLE 12
Group Assignment and Dose Levels

| | | | Dose | Dose | Number of | |
|---|---|---|---|---|---|---|
| | Dose Regimen | | Level^{b} | Concentration | Animals^{c} | |
| Group^{a} | Left Eye | Right Eye | (gc/eye) | (gc/mL) | Males | Females |
| 1 | Control Article 1 | Control Article 1 | 0 | 0 | 1 | NA |
| 2 | Test Article 1 | Test Article 1 | 3 × 10¹¹ | 3 × 10¹² | 3 | 1 |

gc = genome copies
^{a}Group 1 was administered control articles only.
^{b}Dose levels for Groups 1 and 2 were based on a dose volume of 100 μL/eye/dose administered as two 50 μL injections.
^{c}All animals were sacrificed on Day 29 of the dosing phase.

Dose Administration: the preparation of test articles and control articles are shown in Table 13. The test articles and control articles were stored in a freezer between −60° C. and −80° C. and thawed at room temperature on the day of use. The formulations were thawed at room temperature and stored on cold packs until used for syringe filling. Animals were anesthetized with ketamine and dexmedetomidine prior to suprachoroidal injection. In administration, two suprachoroidal injections of 50 μL (Groups 1 and 2) was administered to each eye (between 3 and 4 mm from the limbus) over 10 to 15 seconds. The syringe and microneedle size are shown in Table 13. The first injection in the right eye was administered in the superior temporal quadrant (i.e., between the 10 o'clock and 11 o'clock positions), and the second injection in the right eye (as applicable) was administered in the inferior nasal quadrant (i.e., between the 4 o'clock and 5 o'clock positions). The first injection in the left eye was administered in the superior temporal quadrant (i.e., between the 1 o'clock and 2 o'clock positions), and the second injection in the left eye (as applicable) was administered in the inferior nasal quadrant (i.e., between the 7 o'clock and 8 o'clock positions). Following the injection, the needle was kept in the eye for approximately 30 seconds before being withdrawn. Upon withdrawal of the micro needle, a cotton-tipped applicator (dose wipe) was placed over the injection site for approximately 10 seconds.

TABLE 13
Preparation of Test and Vehicle Control Articles

| | Formulation | Composition | Syringe Preparation |
|---|---|---|---|
| Test | Gel B | In ready-to-use vials containing | 1-mL BD syringe (Part No. |
| Article | Formulation | 1.1 mL at 3 × 10¹² genome copies | 309628) with an attached vial |
| 1 | | (GC)/mL | adapter (West Pharma Services |
| | | | IL, Ltd; Part No. 8070101), and |
| | | | affixed to a MedOne Surgical |
| | | | Suprachoroidal Needle 29 g × |
| | | | 0.7 mm (REF: 8014-16) |
| Control | Placebo Gel B | 18.0% poloxamer 407 and 6.5% | 1-mL BD syringe (Part No. |
| Article | Formulation | poloxamer 188 in modified | 309628) with an attached vial |
| 1 | (vehicle) | DPBS with sucrose (5.84 | adapter (West Pharma Services |
| | | mg/mL sodium chloride, 0.201 | IL, Ltd; Part No. 8070101), and |
| | | mg/mL potassium chloride, 1.15 | affixed to a MedOne Surgical |
| | | mg/mL sodium phosphate | Suprachoroidal Needle 29 g × |
| | | dibasic anhydrous, 0.200 | 0.7 mm (REF: 8014-16) |
| | | mg/mL potassium phosphate | |
| | | monobasic, 40.0 mg/mL (4% | |
| | | w/v) sucrose, 0.001% (0.01 | |
| | | mg/mL) poloxamer 188, pH 7.4 | |

Anti-AAV8-anti-VEGF-ab Transgene Product Antibody Analysis: blood samples were taken as discussed above once predose, and on the day of scheduled sacrifice (Day 29). Serum samples were analyzed for antibodies to the AAV8-anti-VEGF-ab using a validated antibody assay. For AAV8-anti-VEGF-ab transgene analysis, blood samples were taken as described above at least two weeks prior to administration, on Day 15, and on the day of scheduled sacrifice (Day 29). Samples were then analyzed by a validated antibody assay.

Aqueous Humor Collection: approximately 50 μL was removed from each eye at least 2 weeks prior to administration, on Day 15, and on the day the scheduled sacrificed (Day 29). Aqueous humor samples from each eye were placed into separate tubes with Watson barcoded labels, snap frozen in liquid nitrogen, and placed on dry ice until stored between −60° C. and −80° C. Samples were analyzed for anti-VEGF concentration by a validated method.

Termination of Study: animals were anesthetized with sodium pentobarbital and exsanguinated on Day 29. Bone marrow smears were collected on Day 29, and collected (if possible) from animals sacrificed at an unscheduled interval.

Necropsy Collections of Aqueous Humor and Vitreous Humor: Following exsanguination, eyes were enucleated and aqueous humor and vitreous humor samples were collected. Following collection, samples were flash-frozen and stored between −60° C. and −80° C. Aqueous and vitreous samples were analyzed for transgene concentration by a validated method.

Ocular Tissue Collection for Biodistribution: following exsanguination, the right eye from each animal and the left eye from the last two animals (depending on survival) in Group 2 were enucleated and tissues were collected. Tissues were collected into separate tubes. Collected tissue included choroid with retinal pigmented epithelium, retina, and sclera. Tissues were collected using ultra-clean procedures as described above, and rinsed with saline and blotted dry. Samples were flash-frozen and stored between −60° C. and −80° C. Samples were analyzed for vector DNA or RNA using a qPCR or qRT-PCR method.

Comparator study: in a Cynomolgous monkey study conducted analogously to the protocols described in this Example, a control formulation (control article 1.5) was injected to the SCS of the each eye of the subjects (temporal superior and nasal inferior injection with microinjector). The aqueous control formulation does not form a gel.

TABLE 15
Preparation of Control Formulation

| | Formulation | Composition | Syringe Preparation |
|---|---|---|---|
| Control | Control SCS | Modified DPBS with sucrose (5.84 mg/mL | Clearside |
| Article | formulation | sodium chloride, 0.201 mg/mL potassium | microinjector syringe |
| 1.5 | | chloride, 1.15 mg/mL sodium phosphate dibasic | as described in Table 9 |
| | | anhydrous, 0.200 mg/mL potassium phosphate | |
| | | monobasic, 40.0 mg/mL (4% w/v) sucrose, | |
| | | 0.001% (0.01 mg/mL) poloxamer 188, pH 7.4) | |

The control formulation also contained AAV8-anti-VEGF-ab and was dosed at 3×10¹¹ gc/eye in 100 μL/eye/dose (two 50 μL injections).

Data Evaluation and Statistical Analysis: statistical data analyses were calculated using means and standard deviations. Transgene product (protein) in aqueous humor was assessed at 15 and 29 days, otherwise TP, DNA and RNA was assessed in vitreous humor at 29 days.

### RESULTS

TABLE 16
Aqueous Humor Transgene Product (ng/mL)

| | Control Article 1- | | Test Article 1- two | | Control Article 1.5- | |
|---|---|---|---|---|---|---|
| | placebo | | injections | | control formulation | |
| | (TP ng/mL) | | (TP ng/mL) | | (TP ng/mL) | |
| | 15 days | 29 days | 15 days | 29 days | 15 days | 29 days |
| Avg. | 0^{a} | 0^{a} | 16.79 | 29.44 | 2.79 | 3.69 |

^{a}when values were below limit of quantification (<0.100 ng/mL), a value of “0” was assigned for calculation of descriptive statistics.

Two injections of Test article 1 (gel formulation) into the SCS of the eye at the temporal superior and nasal inferior resulted in greater transgene product (TP) concentration in the aqueous humor compared to the Control Formulation.

TABLE 17
Vitreous Humor Transgene Product (ng/mL)

| | Control Article 1- | Test Article 1- | Control Article 1.5- |
|---|---|---|---|
| | placebo | two injections | control formulation |
| | (TP ng/mL) | (TP ng/mL) | (TP ng/mL) |
| Avg. | 0^{a} | 67.76 | 17.99 |

^{a}when values were below limit of quantification (<0.100 ng/mL), a value of “0” was assigned for calculation of descriptive statistics.

Test Article 1 injected into the SCS at the temporal superior and nasal inferior location of the eye results in a greater TP concentration in the VH compared to Control Formulation. Vitreous humor transgene product concentration was higher overall than TP found in aqueous humor at 15 days and 29 days following injection.

TABLE 18
Serum Transgene Product (ng/mL)

| | Control Article 1- | Test Article 1- | Control Article 1.5- |
|---|---|---|---|
| | placebo | two injections | control formulation |
| | (TP ng/mL) | (TP ng/mL) | (TP ng/mL) |
| Avg. | 0^{a} | 0.28 | 0.44 |

^{a}when values were below limit of quantification (<0.100 ng/mL), a value of “0” was assigned for calculation of descriptive statistics.

Each of Test article 1 (gel) or Control formulation containing AAV8-anti-VEGF-ab injected into the SCS produced minimal titers of transgene product (anti-VEGF-ab) in the serum.

TABLE 19
DNA or RNA (copies/μg) Biodistribution in Tissues

| | Control Article | | Test Article 1- | | Control Article 1.5- | |
|---|---|---|---|---|---|---|
| | 1- placebo | | two injections | | control formulation | |
| | (copies/μg) | | (copies/μg) | | (copies/μg) | |
| | DNA | RNA | DNA | RNA | DNA | RNA |
| Retina | nt | nt | 2.08E+04 | 7.15E+05 | 5.61E+03 | nt |
| (Avg.) | | | | | | |
| RPE/ | nt | nt | 1.03E+07 | 1.93E+05 | 3.97E+06 | nt |
| Choroid | | | | | | |
| (Avg.) | | | | | | |
| Sclera | nt | nt | 1.29E+08 | 1.91E+04 | 7.92E+07 | nt |
| (Avg.) | | | | | | |

nt = not tested

Test Article 1 (gel) had an impact on delivery to the retina and choroid, compared to the Control formulation.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1. A pharmaceutical composition suitable for administration to the suprachoroidal space (SCS) of an eye of a human subject, wherein the pharmaceutical composition comprises a recombinant adeno-associated virus (AAV) vector comprising an expression cassette encoding a transgene, and wherein the pharmaceutical has a viscosity and/or higher elastic modulus that increases with increasing temperature.

2. The pharmaceutical composition of claim 1, wherein the composition has a gelation temperature of about 27-32° C.

3. The pharmaceutical composition of claim 1 or 2, wherein the composition has a gelation time of about 15-90 seconds.

4. The pharmaceutical composition of any one of claims 1-3, wherein the composition has a viscosity of about 183 mPas at 5° C. as measured at a shear rate of about 1 s⁻¹ to about 1000 s⁻¹.

5. The pharmaceutical composition of any one of claims 1-3, wherein the composition has a viscosity of less than about 183 mPas at 5° C. as measured at a shear rate of about 1 s⁻¹ to 1000 s⁻¹.

6. The pharmaceutical composition any one of claims 1-3, wherein the composition has a viscosity of about 183 mPas at 20° C. as measured at a shear rate of at about 1 s⁻¹ to 1000 s⁻¹.

7. The pharmaceutical composition of any one of claims 1-3, wherein the composition has a viscosity of less than about 183 mPas at 20° C. as measured at a shear rate of at about 1 s⁻¹ to 1000 s⁻¹

8. The pharmaceutical composition of any one of claims 1-7, wherein, the elastic modulus of a pharmaceutical composition provided herein at under 27° C. is less than about or about 0.1 Pa, less than about or about 0.01 Pa, less than about or about 0.001 Pa or zero.

9. The pharmaceutical composition of any one of claims 1-7, wherein the elastic modulus of a pharmaceutical composition provided herein at 32° C. to 35° C. is about or at least about 0.1 Pa, about or at least about 1 Pa, about or at least about 10 Pa, about or at least about 100 Pa, about or at least about 1000 Pa, about or at least about 10,000 Pa or about or at least about 100,000 Pa.

10. The pharmaceutical composition of any one of claims 1-8, wherein the clearance time after suprachoroidal administration is equal to or greater than the clearance time of a reference pharmaceutical composition after suprachoroidal administration, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a lower viscosity and/or lower elastic modulus than the pharmaceutical composition at about 32-35° C.

11. The pharmaceutical composition of any one of claims 1-8, wherein a circumferential spread after suprachoroidal administration is smaller as compared to a circumferential spread of a reference pharmaceutical composition after suprachoroidal administration, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a lower viscosity and/or lower elastic modulus than the pharmaceutical composition at about 32-35° C.

12. The pharmaceutical composition of claim 11, wherein the circumferential spread after suprachoroidal administration is smaller by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

13. The pharmaceutical composition of any one of claims 1-8, wherein a thickness at a site of injection after suprachoroidal administration is equal to or higher as compared to a thickness at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a lower viscosity and/or lower elastic modulus than the pharmaceutical composition at about 32-35° C.

14. The pharmaceutical composition of any one of claims 1-8, wherein an expression level of the transgene is detected in the eye for a longer period of time after suprachoroidal administration as compared to a period of time that an expression level of the transgene is detected in the eye after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a lower viscosity and/or lower elastic modulus than the pharmaceutical composition at about 32-35° C.

15. The pharmaceutical composition of any one of claims 1-8, wherein the concentration of the transgene in the eye after suprachoroidal administration is equal to or higher as compared to the concentration of the transgene in the eye suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a lower viscosity and/or lower elastic modulus than the pharmaceutical composition at about 32-35° C.

16. The pharmaceutical composition of any one of claims 1-8, wherein the rate of transduction at a site of injection after suprachoroidal administration is equal to or higher as compared to the rate of transduction at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a lower viscosity and/or lower elastic modulus than the pharmaceutical composition at about 32-35° C.

17. The pharmaceutical composition of any one of claims 1-8, wherein a level of VEGF-induced vasodilation and/or vascular leakage after suprachoroidal administration is equal to or decreased as compared to a level of VEGF-induced vasodilation and/or vascular leakage after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a lower viscosity and/or lower elastic modulus than the pharmaceutical composition at about 32-35° C.

18. The pharmaceutical composition of any one of claims 10, 11, and 13-17, wherein the viscosity and/or elastic modulus of the pharmaceutical composition and the viscosity and/or elastic modulus of the reference pharmaceutical composition is measured at the same shear rate.

19. The pharmaceutical composition of any one of claims 4-11 and 13-17, wherein the viscosity and/or elastic modulus of the pharmaceutical composition is measured at a shear rate of at least about 1,000 s⁻¹, 2,000 s⁻¹, 3,000 s⁻¹, 4,000 s⁻¹, 5,000 s⁻¹, 6,000 s⁻¹, 7,000 s⁻¹, 8,000 s⁻¹, 9,000 s⁻¹, 10,000 s⁻¹, 15,000 s⁻¹, 20,000 s⁻¹, or 30,000 s⁻¹.

20. The pharmaceutical composition of any one of claims 1-19, wherein the recombinant AAV is Construct II.

21. The pharmaceutical composition of any one of claims 1-20, wherein the transgene is an anti-human vascular endothelial growth factor (anti-VEGF) antibody.

22. The pharmaceutical composition of any one of claims 1-21, wherein the recombinant AAV comprises components from one or more adeno-associated virus serotypes selected from the group consisting of AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

23. The pharmaceutical composition of any one of claims 1-22, wherein the recombinant AAV is AAV8.

24. The pharmaceutical composition of any one of claims 1-19 and 21-22, wherein the recombinant AAV is AAV9.

25. The pharmaceutical composition of any one of claims 1-24, wherein the pharmaceutical composition comprises sucrose.

26. The pharmaceutical composition of any one of claims 1-24, wherein the pharmaceutical composition does not comprise sucrose.

27. The pharmaceutical composition of any one of claims 1-26, wherein the clearance time after suprachoroidal administration of the pharmaceutical composition is greater by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or at least 500%.

28. The pharmaceutical composition of any one of claims 1-27, wherein the clearance time after suprachoroidal administration of the pharmaceutical composition is of about 30 minutes to about 20 hours, about 2 hours to about 20 hours, about 30 minutes to about 24 hours, about 1 hour to about 2 hours, about 30 minutes to about 90 days, about 30 minutes to about 60 days, about 30 minutes to about 30 days, about 30 minutes to about 21 days, about 30 minutes to about 14 days, about 30 minutes to about 7 days, about 30 minutes to about 3 days, about 30 minutes to about 2 days, about 30 minutes to about 1 day, about 4 hours to about 90 days, about 4 hours to about 60 days, about 4 hours to about 30 days, about 4 hours to about 21 days, about 4 hours to about 14 days, about 4 hours to about 7 days, about 4 hours to about 3 days, about 4 hours to about 2 days, about 4 hours to about 1 day, about 4 hours to about 8 hours, about 4 hours to about 16 hours, about 4 hours to about 20 hours, about 1 day to about 90 days, about 1 day to about 60 days, about 1 day to about 30 days, about 1 day to about 21 days, about 1 day to about 14 days, about 1 day to about 7 days, about 1 day to about 3 days, about 2 days to about 90 days, about 3 days to about 90 days, about 3 days to about 60 days, about 3 days to about 30 days, about 3 days to about 21 days, about 3 days to about 14 days, or about 3 days to about 7 days.

29. The pharmaceutical composition of any one of claims 1-27, wherein the clearance time after suprachoroidal administration of the pharmaceutical composition is not prior to about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

30. The pharmaceutical composition of any one of claims 1-27, wherein the clearance time of the reference pharmaceutical composition after suprachoroidal administration is of at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, or 14 days.

31. The pharmaceutical composition of any one of claims 1-30, wherein the clearance time is from the SCS or from the eye.

32. The pharmaceutical composition of any one of claims 13 and 20-31, wherein the thickness at the site of injection after suprachoroidal administration of the pharmaceutical composition is higher by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

33. The pharmaceutical composition of any one of claims 13 and 20-31, wherein the thickness at the site of injection after suprachoroidal administration of the pharmaceutical composition is about 500 μm to about 3.0 mm, 750 μm to about 2.8 mm, about 750 μm to about 2.5 mm, about 750 μm to about 2 mm, or about 1 mm to about 2 mm.

34. The pharmaceutical composition of any one of claims 13 and 20-31, wherein the thickness at the site of injection after suprachoroidal administration of the pharmaceutical composition is of at least about 50 μm, 100 μm, 200 μm, 300 μm, 400 μm, 500 μm, 600 μm, 700 μm, 800 μm, 900 μm, 1000 μm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm.

35. The pharmaceutical composition of any one of claims 13 and 20-31, wherein the thickness at the site of injection after the suprachoroidal administration of the reference pharmaceutical composition is of at most about 1 nm, 5 nm, 10 nm, 25 nm, 50 nm, 100 nm, 200 nm, 300 nm, 400 nm, 500 nm, 600 nm, 700 nm, 800 nm, 900 nm, 1 μm, 5 μm, 10 μm, 15 μm, 20 μm, 25 μm, 30 μm, 35 μm, 40 μm, 50 μm, 100 μm, 200 μm, 300 μm, 400 μm, 500 μm, 600 μm, 700 μm, 800 μm, 900 μm, or 1000 m.

36. The pharmaceutical composition of any one of claims 13 and 20-31, wherein the thickness at the site of injection after suprachoroidal administration of the pharmaceutical composition persists for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years.

37. The pharmaceutical composition of any one of claims 15 and 20-36, wherein the concentration of the transgene in the eye after suprachoroidal administration of the pharmaceutical composition is higher by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

38. The pharmaceutical composition of any one of claims 14 and 20-37, wherein the longer period of time after suprachoroidal administration of the pharmaceutical composition is longer by at least 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

39. The pharmaceutical composition of any one of claims 1-38, wherein the transgene is detected in the eye after suprachoroidal administration of the pharmaceutical composition for at least about 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

40. The pharmaceutical composition of any one of claims 10-39, wherein the transgene is detected in the eye after suprachoroidal administration of the reference pharmaceutical composition for at most about 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, or 100 days, 120 days, 140 days, 160 days, 180 days, 200 days, 220 days, 240 days, 260 days, 280 days, 300 days, 320 days, 340 days, 360 days, 380 days, or 400 days after.

41. The pharmaceutical composition of claim 21, wherein a level of VEGF-induced vasodilation and/or vascular leakage after suprachoroidal administration of the pharmaceutical composition is equal to or decreased as compared to a level of VEGF-induced vasodilation and/or vascular leakage after suprachoroidal administration of the reference pharmaceutical composition.

42. The pharmaceutical composition of any one of claims 17-41, wherein the level of VEGF-induced vasodilation and/or vascular leakage after suprachoroidal administration of the pharmaceutical composition is decreased by at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

43. The pharmaceutical composition of any one of claims 16 and 20-42, wherein the rate of transduction at the site of injection after suprachoroidal administration of the pharmaceutical composition is higher by at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

44. The pharmaceutical composition of any one of claims 1-43, wherein the recombinant AAV stability is higher in the pharmaceutical composition as compared to the recombinant AAV stability in the reference pharmaceutical composition.

45. The pharmaceutical composition of claim 44, wherein the recombinant AAV stability is determined by infectivity of the recombinant AAV.

46. The pharmaceutical composition of claim 44, wherein the recombinant AAV stability is determined by a level of aggregation of the recombinant AAV.

47. The pharmaceutical composition of claim 44, wherein the recombinant AAV stability is determined by a level of free DNA released by the recombinant AAV.

48. The pharmaceutical composition of claim 47, wherein the pharmaceutical composition comprises about 50% more, about 25% more, about 15% more, about 10% more, about 5% more, about 4% more, about 3% more, about 2% more, about 1% more, about 0% more, about 1% less, about 2% less, about 5% less, about 7% less, about 10% less, about 2 times more, about 3 times more, about 2 times less, about 3 times less, free DNA as compared to a level of free DNA in the reference pharmaceutical composition.

49. The pharmaceutical composition of claim 45, wherein the recombinant AAV in the pharmaceutical composition has an infectivity that is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times higher as compared to the infectivity of the recombinant AAV in the reference pharmaceutical composition.

50. The pharmaceutical composition of claim 46, wherein the pharmaceutical composition comprises at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times less recombinant AAV aggregation as compared to a level of the recombinant AAV aggregation in the reference pharmaceutical composition.

51. The pharmaceutical composition of any one of claims 1-50, wherein the transgene is a transgene suitable to treat, or otherwise ameliorate, prevent or slow the progression of a disease of interest.

52. The pharmaceutical composition of any one of claims 1-51, wherein the human subject is diagnosed with nAMD (wet AMD), dry AMD, retinal vein occlusion (RVO), diabetic macular edema (DME), diabetic retinopathy (DR), glaucoma, non-infectious uveitis, x-linked or Batten disease.

53. The pharmaceutical composition of any one of claims 1-51, wherein the human subject is diagnosed with mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis, or kallikrein-related disease.

54. The pharmaceutical composition of any one of claims 1-19 and 22-53, wherein the AAV encodes Palmitoyl-Protein Thioesterase 1 (PPT1), Tripeptidyl-Peptidase 1 (TPP1), anti-VEGF fusion protein, anti-VEGF antibody or antigen-binding fragment thereof, anti-kallikrein antibody or antigen-binding fragment, anti-TNF antibody or antigen-binding fragment, anti-C3 antibody or antigen-binding fragment, or anti-C5 antibody or antigen-binding fragment.

55. The pharmaceutical composition of any one of claims 1-54, wherein the amount of the recombinant AAV genome copies is based on a vector genome concentration.

56. The pharmaceutical composition of any one of claims 1-54, wherein the amount of the recombinant AAV genome copies is based on genome copies per administration.

57. The pharmaceutical composition of any one of claims 1-54, wherein the amount of the recombinant AAV genome copies is based on total genome copies administered to the human subject.

58. The pharmaceutical composition of claim 56, wherein the genome copies per administration is the genome copies of the recombinant AAV per suprachoroidal administration.

59. The pharmaceutical composition of claim 57, wherein the total genome copies administered is the total genome copies of the recombinant AAV administered suprachoroidally.

60. The pharmaceutical composition of claim 55, wherein the vector genome concentration (VGC) is of about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 6.2×10¹¹ GC/mL, about 1×10¹² GC/mL, about 2.5×10¹² GC/mL, about 3×10¹² GC/mL, about 5×10¹² GC/mL, about 6×10¹² GC/mL, about 1.5×10¹³ GC/mL, about 2×10¹³ GC/mL, or about 3×10¹³ GC/mL.

61. The pharmaceutical composition of any one of claims 57 and 59, wherein the total number of genome copies administered is about 6.0×10¹⁰ genome copies, about 1.6×10¹¹ genome copies, about 2.5×10¹¹ genome copies, about 3×10¹¹ genome copies, about 5.0×10¹¹ genome copies, about 6×10¹¹ genome copies, about 3×10¹² genome copies, about 1.0×10¹² genome copies, about 1.5×10¹² genome copies, about 2.5×10¹² genome copies, or about 3.0×10¹³ genome copies.

62. The pharmaceutical composition of any one of claims 56 and 58, wherein the total number of genome copies administered is about 6.0×10¹⁰ genome copies, about 1.6×10¹¹ genome copies, about 2.5×10¹¹ genome copies, about 3×10¹¹ genome copies, about 5.0×10¹¹ genome copies, about 6×10¹¹ genome copies, about 3×10¹² genome copies, about 1.0×10¹² genome copies, about 1.5×10¹² genome copies, about 2.5×10¹² genome copies, or about 3.0×10¹³ genome copies.

63. The pharmaceutical composition of any one of claims 1-62, wherein the pharmaceutical composition is administered once, twice, three times, four times, five times, six times, seven times, eight times, nine times, ten times, fifteen times, twenty times, twenty five times, or thirty times.

64. The pharmaceutical composition of any one of claims 10-63, wherein the reference pharmaceutical composition is administered once, twice, three times, four times, five times, six times, seven times, eight times, nine times, ten times, fifteen times, twenty times, twenty five times, or thirty times.

65. The pharmaceutical composition of any one of claims 1-64, wherein the pharmaceutical composition is administered once in one day, twice in one day, three times in one day, four times in one day, five times in one day, six times in one day, or seven times in one day.

66. The pharmaceutical composition of any one of claims 10-64, wherein the reference pharmaceutical composition is administered once in one day, twice in one day, three times in one day, four times in one day, five times in one day, six times in one day, or seven times in one day.

67. The pharmaceutical composition of any one of claims 1-66, wherein the pharmaceutical composition contains poloxamer 407 and poloxamer 188.

68. The pharmaceutical composition of any one of claims 1-67, wherein the composition comprises 16-22% poloxamer 407.

69. The pharmaceutical composition of any one of claims 1-67, wherein the composition comprises 0-16% poloxamer 188.

70. The pharmaceutical composition of any one of claims 1-67, wherein the composition comprises 19% poloxamer 407 and 6% poloxamer 188.

71. The pharmaceutical composition of any one of claims 1-67, wherein the composition comprises 18% poloxamer 407 and 6.5% poloxamer 188.

72. The pharmaceutical composition of any one of claims 1-67, wherein the composition comprises 17.5% poloxamer 407 and 7% poloxamer 188

73. The pharmaceutical composition of any one of claims 1-72, wherein the composition comprises modified Dulbecco's phosphate-buffered saline solution, and optionally a surfactant.

74. The pharmaceutical composition of any one of claims 1-72, wherein the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, and optionally a surfactant.

75. The pharmaceutical composition of any one of claims 1-72, wherein the composition comprises potassium chloride, potassium phosphate monobasic, sodium chloride, sodium phosphate dibasic anhydrous, sucrose, and optionally a surfactant.

76. A method of treating a disease in a subject, the method comprising administering the pharmaceutical composition of any one of claims 1-72.

77. A method of treating a disease in a subject, the method comprising administering the pharmaceutical composition of claim 4 or 5 to the subject, wherein the pharmaceutical composition is at a temperature of about 2-10° C. when being administered.

78. A method of treating a disease in a subject, the method comprising administering the pharmaceutical composition of claim 6 or 8 to the subject, wherein the pharmaceutical composition is at a temperature of about 20-25° C. when being administered.

79. The method of any one of claims 76-78, wherein the pharmaceutical composition is administered with an injection pressure of less than about 43 PSI.

80. The method of any one of claims 76-78, wherein the pharmaceutical composition is administered with an injection pressure of less than about 65 PSI.

81. The method of any one of claims 76-78, wherein the pharmaceutical composition is administered with an injection pressure of less than about 100 PSI.

82. The method of any one of claims 76-81, wherein the pharmaceutical composition is administered using a 29 gauge needle.

83. The method of any one of claims 76-81, wherein the pharmaceutical composition is administered using a 30 gauge needle.

84. The method of any one of claims 76-83, wherein the pharmaceutical composition is administered in an injection time of about 10-15 seconds.

85. The method of any one of claims 76-83, wherein the pharmaceutical composition is administered in an injection time of about 5-30 seconds.

86. The method of any one of claims 76-85, wherein the subject is human.

87. The method of any one of claims 76-86, wherein the disease is selected from the group consisting of nAMD (wet AMD), dry AMD, retinal vein occlusion (RVO), diabetic macular edema (DME), diabetic retinopathy (DR), Batten disease, mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis and kallikrein-related disease.


SEQUENCE LISTING (55 sequences)

SEQ ID NO: 1 - protein, 214 aa, Artificial Sequence
  Ranibizumab Fab Amino Acid Sequence - light chain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 231 aa, Artificial Sequence
  Ranibizumab Fab Amino Acid Sequence - heavy chain
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 3 - protein, 214 aa, Artificial Sequence
  Bevacizumab Fab Amino Acid Sequence - Light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 231 aa, Artificial Sequence
  Bevacizumab Fab Amino Acid Sequence - Heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 5 - protein, 26 aa, Artificial Sequence
  VEGF-A signal peptide
MNFLLSWVHWSLALLLYLHHAKWSQA

SEQ ID NO: 6 - protein, 29 aa, Artificial Sequence
  Fibulin-1 signal peptide
MERAAPSRRVPLPLLLLGGLALLAAGVDA

SEQ ID NO: 7 - protein, 19 aa, Artificial Sequence
  Vitronectin signal peptide
MAPLRPLLILALLAWVALA

SEQ ID NO: 8 - protein, 18 aa, Artificial Sequence
  Complement Factor H signal peptide
MRLLAKIICLMLWAICVA

SEQ ID NO: 9 - protein, 19 aa, Artificial Sequence
  Opticin signal peptide
MRLLAFLSLLALVLQETGT

SEQ ID NO: 10 - DNA, 728 bp, Artificial Sequence
  Bevacizumab cDNA - Light chain
gctagcgccaccatgggctggtcctgcatcatcctgttcctggtggccaccgccaccggcgtgcactccgacatccagatgacccagtccccctcctccctgtccgcctccgtgggcgaccgggtgaccatcacctgctccgcctcccaggacatctccaactacctgaactggtaccagcagaagcccggcaaggcccccaaggtgctgatctacttcacctcctccctgcactccggcgtgccctcccggttctccggctccggctccggcaccgacttcaccctgaccatctcctccctgcagcccgaggacttcgccacctactactgccagcagtactccaccgtgccctggaccttcggccagggcaccaaggtggagatcaagcggaccgtggccgccccctccgtgttcatcttccccccctccgacgagcagctgaagtccggcaccgcctccgtggtgtgcctgctgaacaacttctacccccgggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccggggcgagtgctgagcggccgcctcgag

SEQ ID NO: 11 - DNA, 1440 bp, Artificial Sequence
  Bevacizumab cDNA - heavy chain
gctagcgccaccatgggctggtcctgcatcatcctgttcctggtggccaccgccaccggcgtgcactccgaggtgcagctggtggagtccggcggcggcctggtgcagcccggcggctccctgcggctgtcctgcgccgcctccggctacaccttcaccaactacggcatgaactgggtgcggcaggcccccggcaagggcctggagtgggtgggctggatcaacacctacaccggcgagcccacctacgccgccgacttcaagcggcggttcaccttctccctggacacctccaagtccaccgcctacctgcagatgaactccctgcgggccgaggacaccgccgtgtactactgcgccaagtacccccactactacggctcctcccactggtacttcgacgtgtggggccagggcaccctggtgaccgtgtcctccgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcacccagacctacatctgcaacgtgaaccacaagccctccaacaccaaggtggacaagaaggtggagcccaagtcctgcgacaagacccacacctgccccccctgccccgcccccgagctgctgggcggcccctccgtgttcctgttcccccccaagcccaaggacaccctgatgatctcccggacccccgaggtgacctgcgtggtggtggacgtgtcccacgaggaccccgaggtgaagttcaactggtacgtggacggcgtggaggtgcacaacgccaagaccaagccccgggaggagcagtacaactccacctaccgggtggtgtccgtgctgaccgtgctgcaccaggactggctgaacggcaaggagtacaagtgcaaggtgtccaacaaggccctgcccgcccccatcgagaagaccatctccaaggccaagggccagccccgggagccccaggtgtacaccctgcccccctcccgggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgtcccccggcaagtgagcggccgcc

SEQ ID NO: 12 - DNA, 733 bp, Artificial Sequence
  Ranibizumab cDNA (Light chain comprising a signal sequence)
gagctccatggagtttttcaaaaagacggcacttgccgcactggttatgggttttagtggtgcagcattggccgatatccagctgacccagagcccgagcagcctgagcgcaagcgttggtgatcgtgttaccattacctgtagcgcaagccaggatattagcaattatctgaattggtatcagcagaaaccgggtaaagcaccgaaagttctgatttattttaccagcagcctgcatagcggtgttccgagccgttttagcggtagcggtagtggcaccgattttaccctgaccattagcagcctgcagccggaagattttgcaacctattattgtcagcagtatagcaccgttccgtggacctttggtcagggcaccaaagttgaaattaaacgtaccgttgcagcaccgagcgtttttatttttccgcctagtgatgaacagctgaaaagcggcaccgcaagcgttgtttgtctgctgaataatttttatccgcgtgaagcaaaagtgcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagttttaatcgtggcgaatgctaatagaagcttggtacc

SEQ ID NO: 13 - DNA, 779 bp, Artificial Sequence
  Ranibizumab cDNA (Heavy chain comprising a signal sequence)
gagctcatatgaaatacctgctgccgaccgctgctgctggtctgctgctcctcgctgcccagccggcgatggccgaagttcagctggttgaaagcggtggtggtctggttcagcctggtggtagcctgcgtctgagctgtgcagcaagcggttatgattttacccattatggtatgaattgggttcgtcaggcaccgggtaaaggtctggaatgggttggttggattaatacctataccggtgaaccgacctatgcagcagattttaaacgtcgttttacctttagcctggataccagcaaaagcaccgcatatctgcagatgaatagcctgcgtgcagaagataccgcagtttattattgtgccaaatatccgtattactatggcaccagccactggtatttcgatgtttggggtcagggcaccctggttaccgttagcagcgcaagcaccaaaggtccgagcgtttttccgctggcaccgagcagcaaaagtaccagcggtggcacagcagcactgggttgtctggttaaagattattttccggaaccggttaccgtgagctggaatagcggtgcactgaccagcggtgttcatacctttccggcagttctgcagagcagcggtctgtatagcctgagcagcgttgttaccgttccgagcagcagcctgggcacccagacctatatttgtaatgttaatcataaaccgagcaataccaaagtggataaaaaagttgagccgaaaagctgcgataaaacccatctgtaatagggtacc

SEQ ID NO: 14 - protein, 11 aa, Artificial Sequence
  Bevacizumab and Ranibizumab Light Chain CDR1
SASQDISNYLN

SEQ ID NO: 15 - protein, 7 aa, Artificial Sequence
  Bevacizumab and Ranibizumab Light Chain CDR2
FTSSLHS

SEQ ID NO: 16 - protein, 9 aa, Artificial Sequence
  Bevacizumab and Ranibizumab Light Chain CDR3
QQYSTVPWT

SEQ ID NO: 17 - protein, 10 aa, Artificial Sequence
  Bevacizumab Heavy Chain CDR1
GYTFTNYGMN

SEQ ID NO: 18 - protein, 17 aa, Artificial Sequence
  Bevacizumab Heavy Chain CDR2
WINTYTGEPTYAADFKR

SEQ ID NO: 19 - protein, 14 aa, Artificial Sequence
  Bevacizumab Heavy Chain CDR3
YPHYYGSSHWYFDV

SEQ ID NO: 20 - protein, 10 aa, Artificial Sequence
  Ranibizumab Heavy Chain CDR1
GYDFTHYGMN

SEQ ID NO: 21 - protein, 14 aa, Artificial Sequence
  Ranibizumab Heavy Chain CDR3
YPYYYGTSHWYFDV

SEQ ID NO: 22 - protein, 18 aa, Artificial Sequence
  Albumin signal peptide
MKWVTFISLLFLFSSAYS

SEQ ID NO: 23 - protein, 18 aa, Artificial Sequence
  Chymotrypsinogen signal peptide
MAFLWLLSCWALLGTTFG

SEQ ID NO: 24 - protein, 20 aa, Artificial Sequence
  Interleukin-2 signal peptide
MYRMQLLSCIALILALVTNS

SEQ ID NO: 25 - protein, 15 aa, Artificial Sequence
  Trypsinogen-2 signal peptide
MNLLLILTFVAAAVA

SEQ ID NO: 26 - protein, 19 aa, Artificial Sequence
  F2A site
LLNFDLLKLAGDVESNPGP

SEQ ID NO: 27 - protein, 21 aa, Artificial Sequence
  Linker T2A
GSGEGRGSLLTCGDVEENPGP

SEQ ID NO: 28 - protein, 22 aa, Artificial Sequence
  Linker P2A
GSGATNFSLLKQAGDVEENPGP

SEQ ID NO: 29 - protein, 23 aa, Artificial Sequence
  Linker E2A
GSGQCTNYALLKLAGDVESNPGP

SEQ ID NO: 30 - protein, 25 aa, Artificial Sequence
  Linker F2A
GSGVKQTLNFDLLKLAGDVESNPGP

SEQ ID NO: 31 - protein, 4 aa, Artificial Sequence
  Furin linker
RKRR

SEQ ID NO: 32 - protein, 4 aa, Artificial Sequence
  Furin linker
RRRR

SEQ ID NO: 33 - protein, 4 aa, Artificial Sequence
  Furin linker
RRKR

SEQ ID NO: 34 - protein, 4 aa, Artificial Sequence
  Furin linker
RKKR

SEQ ID NO: 35 - protein, 4 aa, Artificial Sequence
  Furin linker
  MISC_FEATURE (2)..(2): X = any amino acid
  MISC_FEATURE (3)..(3): X = Lys or Arg
RXXR

SEQ ID NO: 36 - protein, 4 aa, Artificial Sequence
  Furin linker
  MISC_FEATURE (2)..(2): X = any amino acid
RXKR

SEQ ID NO: 37 - protein, 4 aa, Artificial Sequence
  Furin linker
  MISC_FEATURE (2)..(2): X = any amino acid
RXRR

SEQ ID NO: 38 - protein, 215 aa, Artificial Sequence
  Ranibizumab Fab Amino Acid Sequence - light chain
MDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 236 aa, Artificial Sequence
  Ranibizumab Fab Amino Acid Sequence - heavy chain
MEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLRKRR

SEQ ID NO: 40 - protein, 232 aa, Artificial Sequence
  Ranibizumab Fab Amino Acid Sequence - heavy chain
MEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 41 - protein, 736 aa, Artificial Sequence
  AAV1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 42 - protein, 735 aa, Artificial Sequence [sequence omitted]

SEQ ID NO: 43 - protein, 736 aa, Artificial Sequence
  AAV3-3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 44 - protein, 734 aa, Artificial Sequence
  AAV4-4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 45 - protein, 724 aa, Artificial Sequence
  AAV5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 46 - protein, 736 aa, Artificial Sequence
  AAV6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 47 - protein, 737 aa, Artificial Sequence
  AAV7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 48 - protein, 738 aa, Artificial Sequence
  AAV8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 49 - protein, 736 aa, Artificial Sequence
  hu31
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 50 - protein, 736 aa, Artificial Sequence
  hu32
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - protein, 736 aa, Artificial Sequence
  AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 52 - protein, 191 aa, Artificial Sequence
  Vascular endothelial growth factor (vegf) Caa44447.1
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 53 - protein, 306 aa, Artificial Sequence
  Palmitoyl-protein thioesterase 1 (ppt1) Aah08426.1
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 54 - protein, 563 aa, Artificial Sequence
  Tripeptidyl-peptidase 1 (tpp1) Np_000382.3
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 55 - protein, 20 aa, Artificial Sequence
  Mutant interleukin-2 signal peptide
MYRMQLLLLIALSLALVTNS